(19) United States
(12) Patent Application Publication
(10) Pub. No.: US 2017/0011661 A1
(43) Pub. Date: Jan. 12, 2017

(54) Greet cards with prepaid gift card holder

(71) Applicant: Steven Patrick Pascale, San Clemente, CA (US)

(72) Inventor: Steven Patrick Pascale, San Clemente, CA (US)

(21) Appl. No.: 14/545,900

(22) Filed: Jul. 6, 2015

(51) Int. Cl. G09F 1/08; A61L 9/14; A61L 9/12

(52) U.S. Cl. CPC G09F 1/08; A61L 9/12; A61L 9/14

(57) ABSTRACT

The present invention primarily relates to greeting cards specifically made for children of all ages. Each card has a mascot motif theme suitable for celebrating special occasions. Collectively, there are 70 unique shapes and sizes. A second aspect of the invention is Christmas ornament capable of holding a pre-paid gift card. Thirdly, the novelty also reveals an apparatus for dispersing perfume or cologne mist spray in an automobile. A fourth embodiment can be used as a hotel key card-presenter, given to patrons “checking-in” and may indeed be ideal when coupled with a wet erase back surface used for noting beverage and snack consumption. And finally, a fifth embodiment which usees the apparatus as a greeting card+a baseball collectible card (as well as other sport(s) collectible cards) and primarily used to present a pre-paid gift card or tickets to a specific team promotion(s).

In the aforementioned embodiments, each illustrates and describes an apparatus inventiveness which includes a base portion front surface as well as back surface. Generally speaking, a “front surface” consists of a planar panel consisting of art work as well as indicia with an aperture for attaching a decorative string-like material, tassel, hook, loop etc. Equally, a back surface capable of writing thereon, magnet, and retaining mechanism used for holding a pre-paid gift card.

### BACKGROUND OF THE INVENTION

1. Field of the Invention

The present invention relates generally to greeting cards with an integrated holder for pre-paid gift card /key card, collectible sports card, as well as vehicle atomizer cards. Still, and more particularly, the present invention relates to an innovative Christmas ornament with pre-paid gift card pocket or die cut retention mechanism, a perfume-cologne dispersing method, as well as a furniture coaster.

While pre-paid gift cards are becoming more and more popular, most greeting cards used to facilitate a pre-paid card pocket are typically inadequate in ergonomic functionality and more importantly . . . are not adequately designed in view of a younger audience. Notably, kids of all ages tend to like similar retail stores and they also enjoy shopping for gifts “on-line” that are of specific interest. For example, one instance are video games that he/she enjoys playing. In light of our new reality, market research indicates that adults are obviously relieved of the burden of selecting an item that may not in line with their childs preferences. Notably, a new era of technology advancements continues to provide fundamental “change” in the way that traditional retail shopping occurs. Yesteryear's gifts, gift certificates, and greeting cards once thought of as “common-place” may very well be a thing of the past. For now, even food and beverage company's like Starbuck's are evolving with pre-paid gift card technology and have the capability to avoid having to issue new or replacement gift certificates.

2. Description of the Prior Art

For many years, greeting cards and greeting cards with pre-paid card holders have been made in a certain manner. In most cases, the card stock utilized continues to be that of a thin stock with perceptions of quality issues and sized approximately 5″×7″. Further, the existing cards are scored (folded) forcing a young recipient (with a limited attention span) to open to the inside page. What's more, the art work for kids is mediocre at best and typically includes printed message considered trite.

While these arrangements worked at one time, it is desirable to improve upon the aesthetical appearance of greeting cards as well as greeting cards with pre-paid gift card holdes. The aim is to make greeting cards for kids ages 5-19 more appealing. By doing so, pre-paid gift card sales should increase this targeted age bracket. Even more profound, children for the first time will feel better about receiving greeting cards as a whole.

The purpose of this invention is to provide greeting cards with pre-paid gift card holders that have (generally speaking) mascot motif themes and shapes.

While this invention has been described in terms of several preferred embodiments, there are of course other alterations, permutation-combinations, and equivalents possibilities. Notably, these and other improvements fall within the scope of this invention. In actuality, it should be noted that there are many alternative ways of producing a greeting card capable of holding a pre-paid gift card.

Greeting cards with pre-paid gift card holders are generally known in the art.

U.S. Pat. No. 7,938,279 provides a gift card carrier that includes a base portion with an opening adapted to receive and support a gift card, which travels through the opening until it engages the base portion. The unfolding of the base portion causes the support portion to unfold, which forces the card back through the opening and outward, thus creating a pop-up effect.

U.S. Pat. No. 6,877,263 affords an integrated greeting card and gft card holder and a holder for a securing a transaction card. The greeting card includes a cover which covers a given gift card placed in the carrier, so that the card is adjustable when in a closed configuration in addition to a second position while the greeting card is in an opened state.

U.S. Pat. No. 6,732,459 supplies a gift package which includes a greeting card and a transaction card carrier similar to those issued by various retail stores and restaurants.

U.S. Pat. No. 8,701,315 presents a gift card holder which couples a pocket for storing a gift card. Importantly, it allows for a tear-away panel for holding a gift card prior to purchase.

U.S. Pat. No. 8,499,478 conveys a transaction card holder including a pop-up element for holding a gift card when the card holder is opened.

U.S. Pat. No. 8,646,606 furnishes an apparatus for holding a gift card while providing graphical, mechanical and audio enhancements.

U.S. Pat. No. 8,800,758 offers a greeting card together with a gift card holder system.

U.S. Pat. No. 8,701,315 proposes a transaction card carrier that includes a pocket for storing a gift card and a tear away panel for holding a gift card prior to purchase.

U.S. Pat. No. 8,707,594 imparts a transaction card carrier for holding numerous gift cards.

U.S. Pat. No. 8,474,162 grants by its design a transaction card carrie including a pop-up element for holding and presenting a card when the card holder is opened. An ornament gift card holder that includes a removably attached gift card, a slot for inserting a gift card into the holder after removal for loading at the point of purchase, a means for hanging the holder upon a Christmas tree, and audio circuitry for recording and playing sound.

U.S. Pat. No. 7,011,249 makes a gift card carrier available with a detachable tab joined to a first panel and to a second panel taken together collectively comprises a gift card holder.

U.S. Pat. No. 7,622,073 offers an apparatus for dispensing active materials comprises a housing and control circuitry disposed within the housing.

Prior patents examples noted above are generally useful for limited intended purpose. Nevertheless, the need remains for greeting cards to have more functional use i.e. greeting card coupled with transaction card carrier, Christmas ornament with pre-paid gift card holder; one which provides an ability of improved graphic themes, innovative gift card carrier design, ability to attach a tassel, magnetic—for attaching to a refrigerator, as well as bringing secondary utility functionality like that found with an atomizer card and sports trading card.

### BRIEF SUMMARY OF THE INVENTION

The present invention primarily integrates a greeting card with a transactional pre-paid gift card carrier. Specifically, the purpose of the invention is to provide 70 different die-cut greeting card shapes; collectively, representing approximately 100 different mascot motif themes and Christmas designs.

In other preferred applications of the invention, the greeting card is a Christmas tree ornament with pre-paid gift card holder, an automobile atomizer card, a hotel key transactional presenter/beverage consumption board, as well as greeting cards capable of retaining collectibla baseball cards with a specific sport team (city) promotion.

Each card comprises of both a front and back planar panel.

The invention is intended to utilize multiple sized envelopes.

These and other features and advantages of the present invention will become more apparent upon reading the following detailed description and studying various illustrations.

### BRIEF DESCRIPTION OF THE DRAWINGS

FIG. 1 illustrates an embodiment of the present invention/die cut clip flaps, ornament ring, neodymium magnet, perfume mist sprayer with glue dot, atomizer canister and chain, tassel, external card with attached sprayer for quick insertion into apparatus, and wet-erase (non permanent) marker.

FIG. 2 is a back view of a frog greeting card with poly pocket, magnet, and aperture with easy release mechanism used for hanging purposes and/or attaching a decorative item like a tassel.

FIG. 3 is a back view of a navy seal greeting card with poly pocket, magnet, and aperture with easy release mechanism used for hanging purposes and/or attaching a decorative item like a tassel.

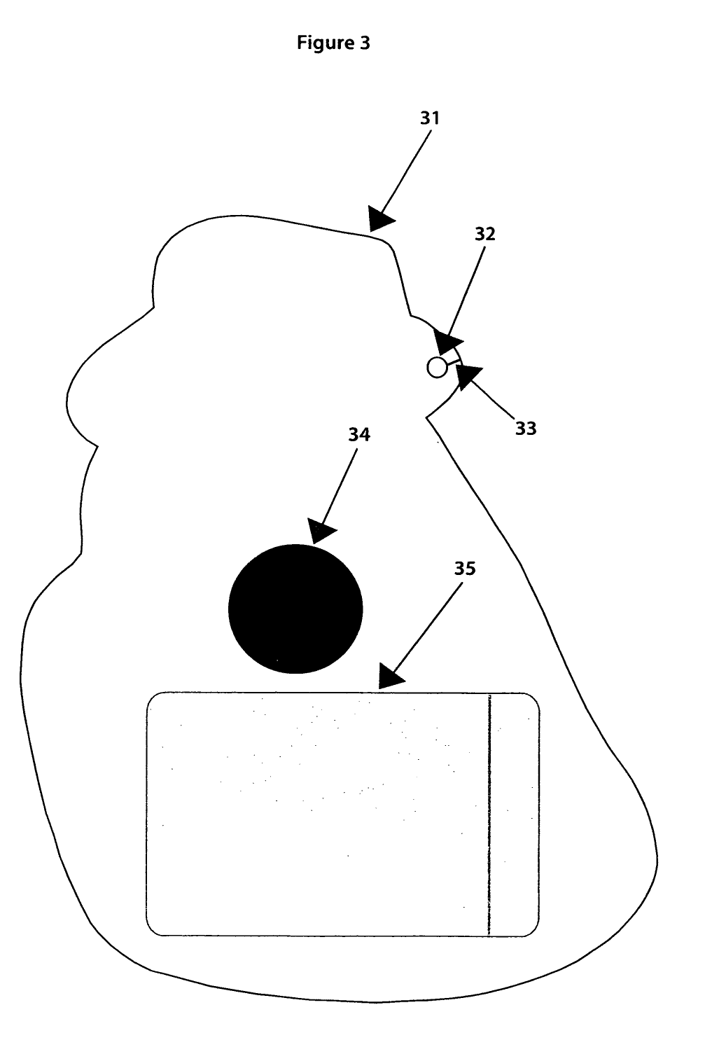

FIG. 4 is a back view of a coffee cup greeting card with poly pocket, magnet, and aperture with easy release mechanism used for hanging purposes and/or attaching a decorative item like a tassel.

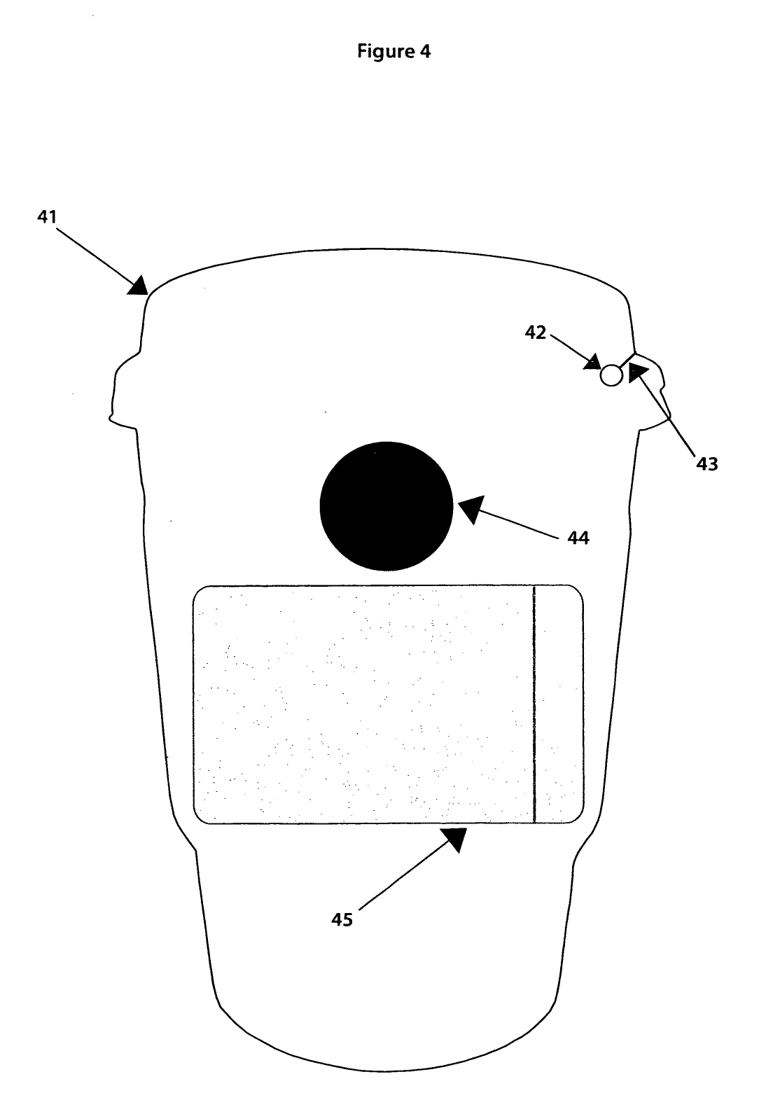

FIG. 5 is a back view of a tiger, African lion, snow leopard greeting cards with poly pocket, magnet, and aperture with easy release mechanism used for hanging purposes and/or attaching a decorative item like a tassel.

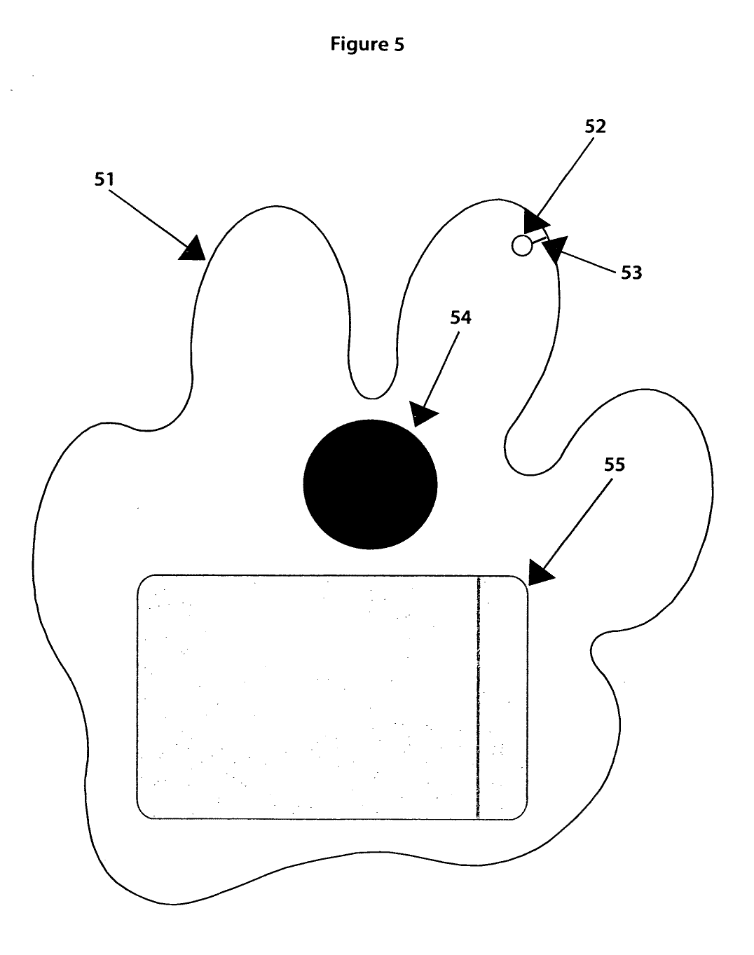

FIG. 6 is a back view of a knight greeting card with poly pocket, magnet, and aperture with easy release mechanism used for hanging purposes and/or attaching a decorative item like a tassel.

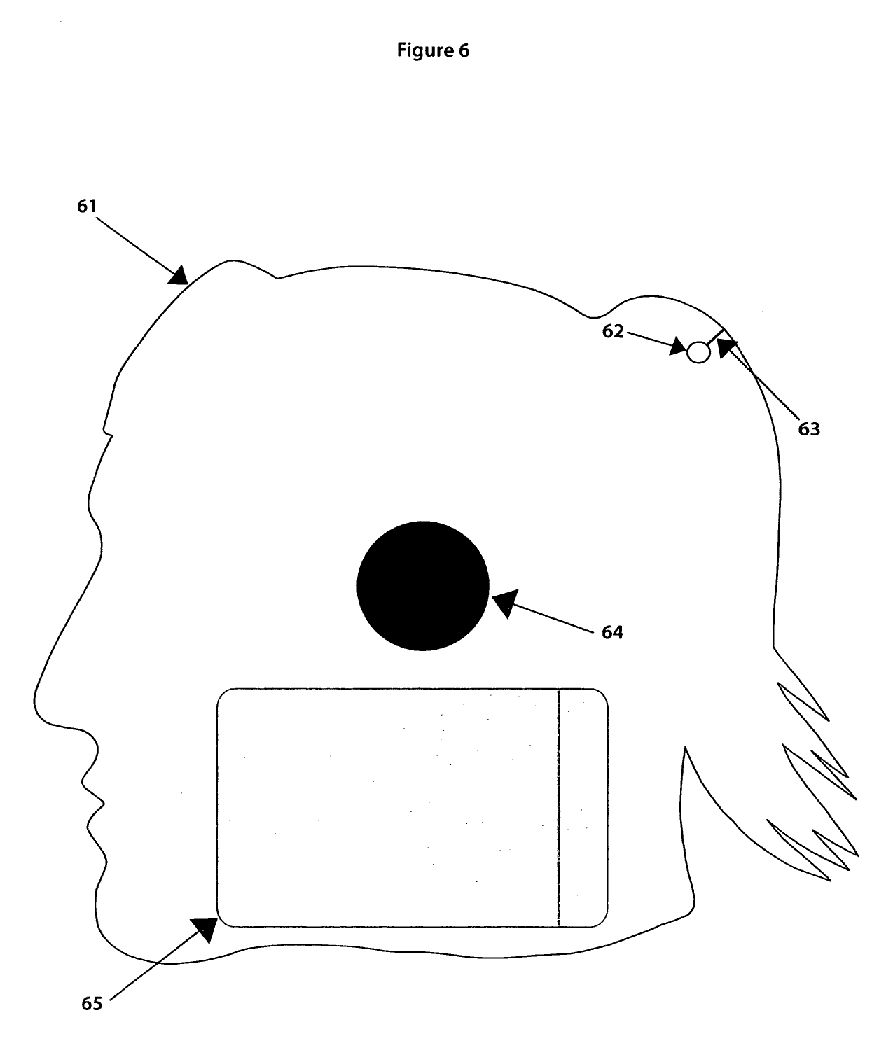

FIG. 7 is a back view of a trojan greeting card with poly pocket, magnet, and aperture with easy release mechanism used for hanging purposes and/or attaching a decorative item like a tassel.

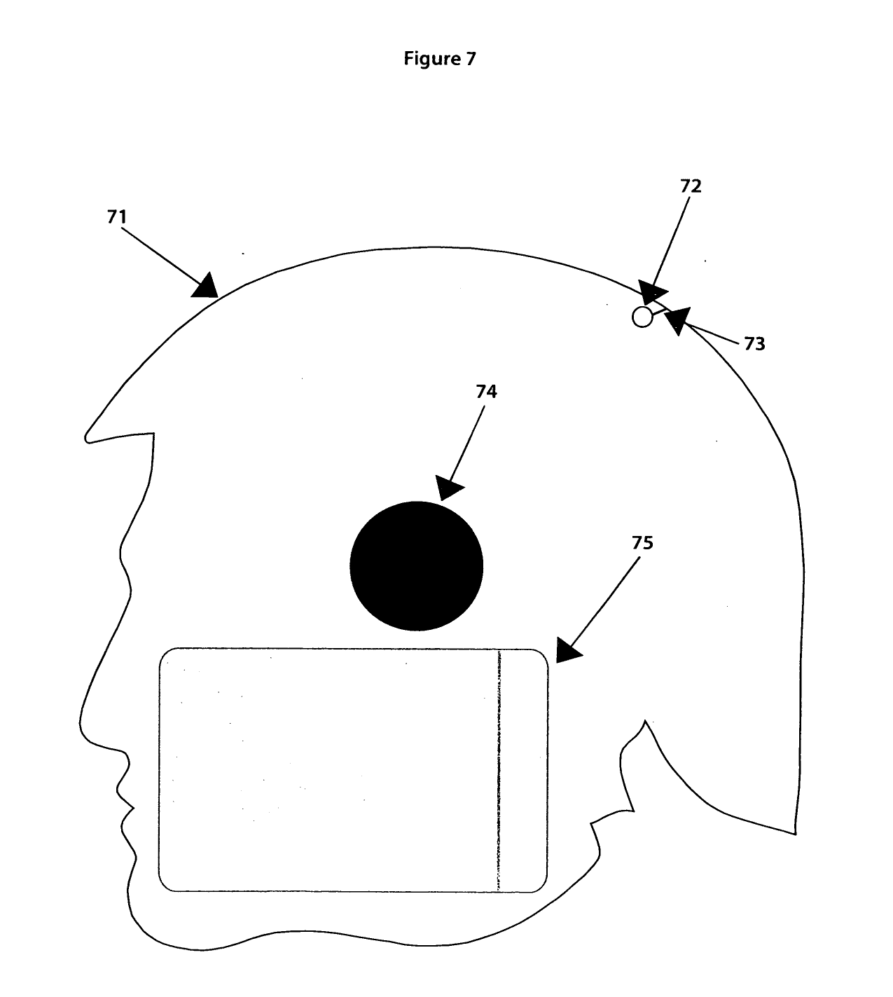

FIG. 8 is a back view of a polar bear greeting card with poly pocket, magnet, and aperture with easy release mechanism used for hanging purposes and/or attaching a decorative item like a tassel.

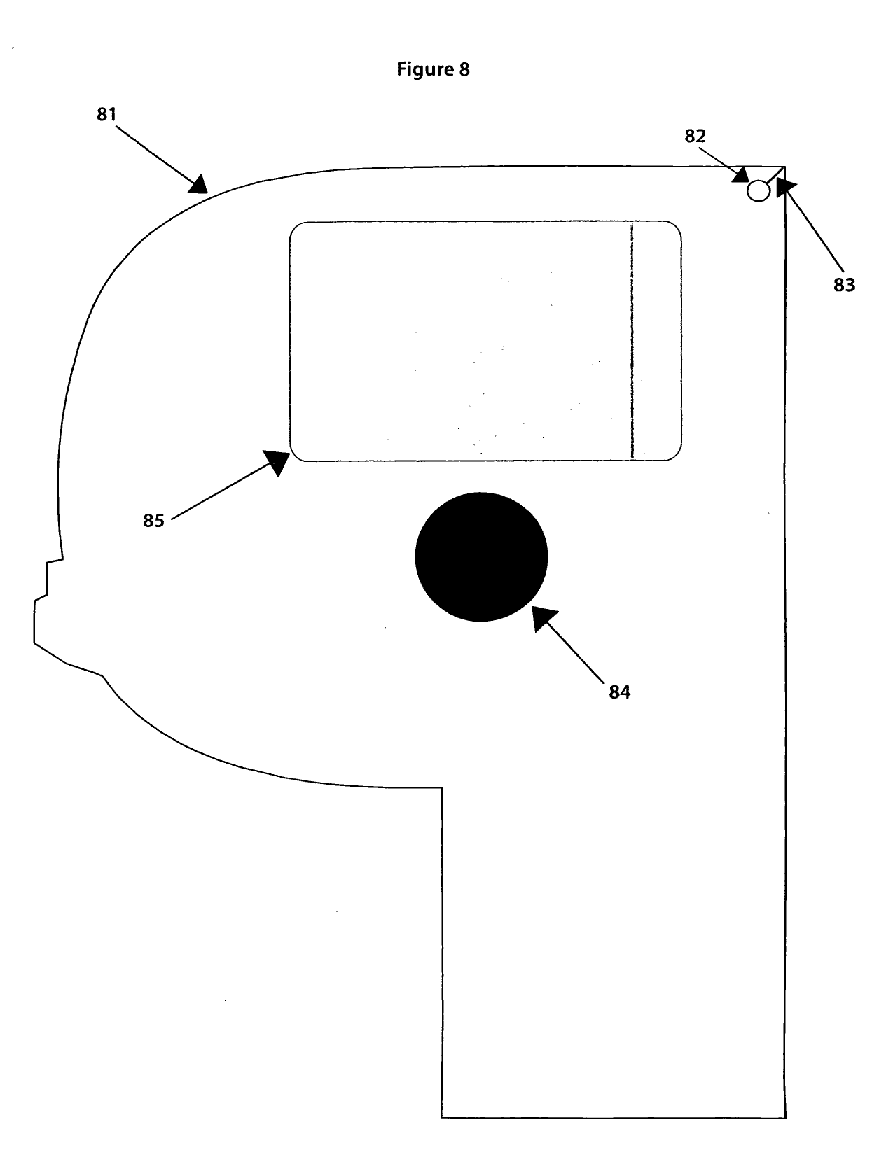

FIG. 9 is a back view of a penguin greeting card with poly pocket, magnet, and aperture with easy release mechanism used for hanging purposes and/or attaching a decorative item like a tassel.

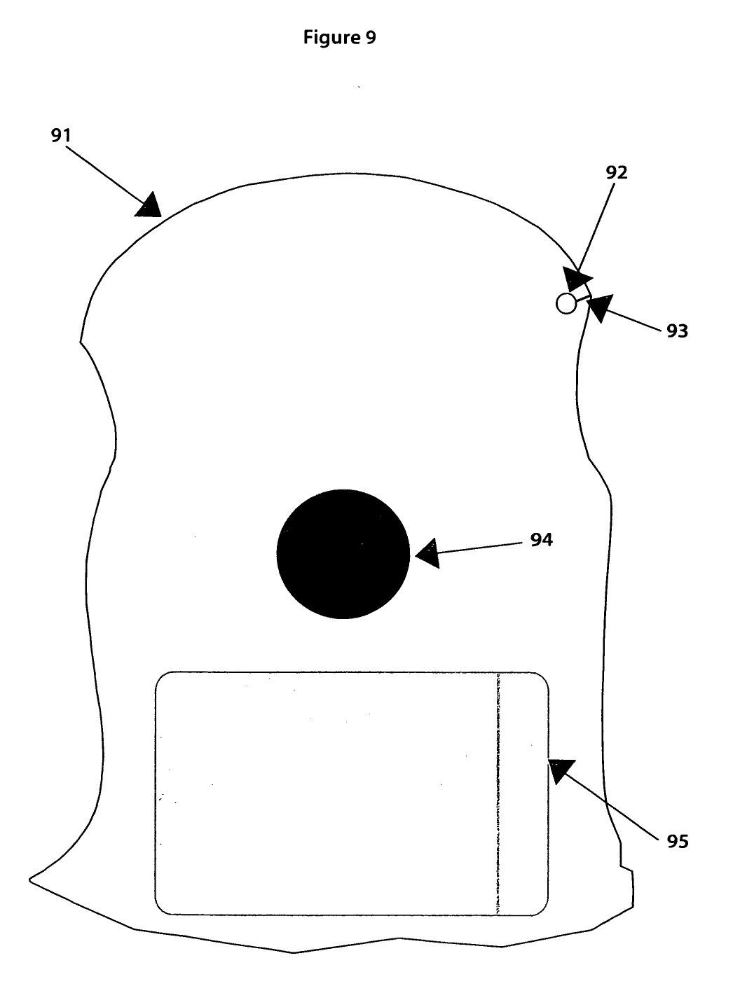

FIG. 10 is a back view of a huskie greeting card with poly pocket, magnet, and aperture with easy release mechanism used for hanging purposes and/or attaching a decorative item like a tassel.

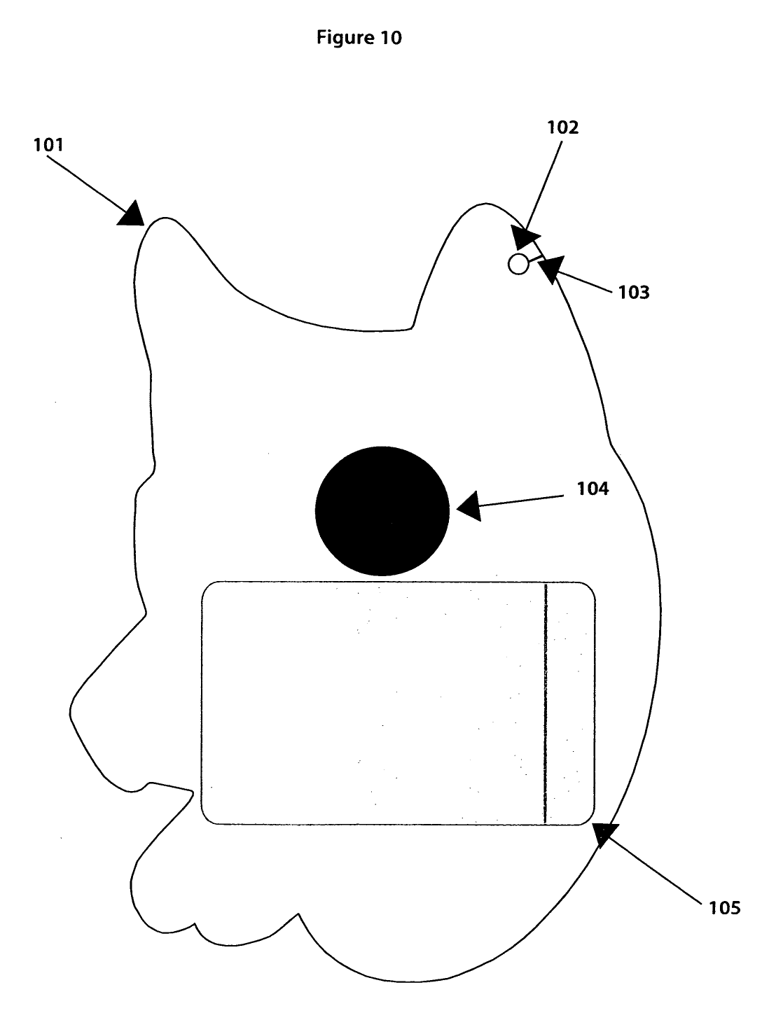

FIG. 11 is a back view of a walrus greeting card with poly pocket, magnet, and aperture with easy release mechanism used for hanging purposes and/or attaching a decorative item like a tassel.

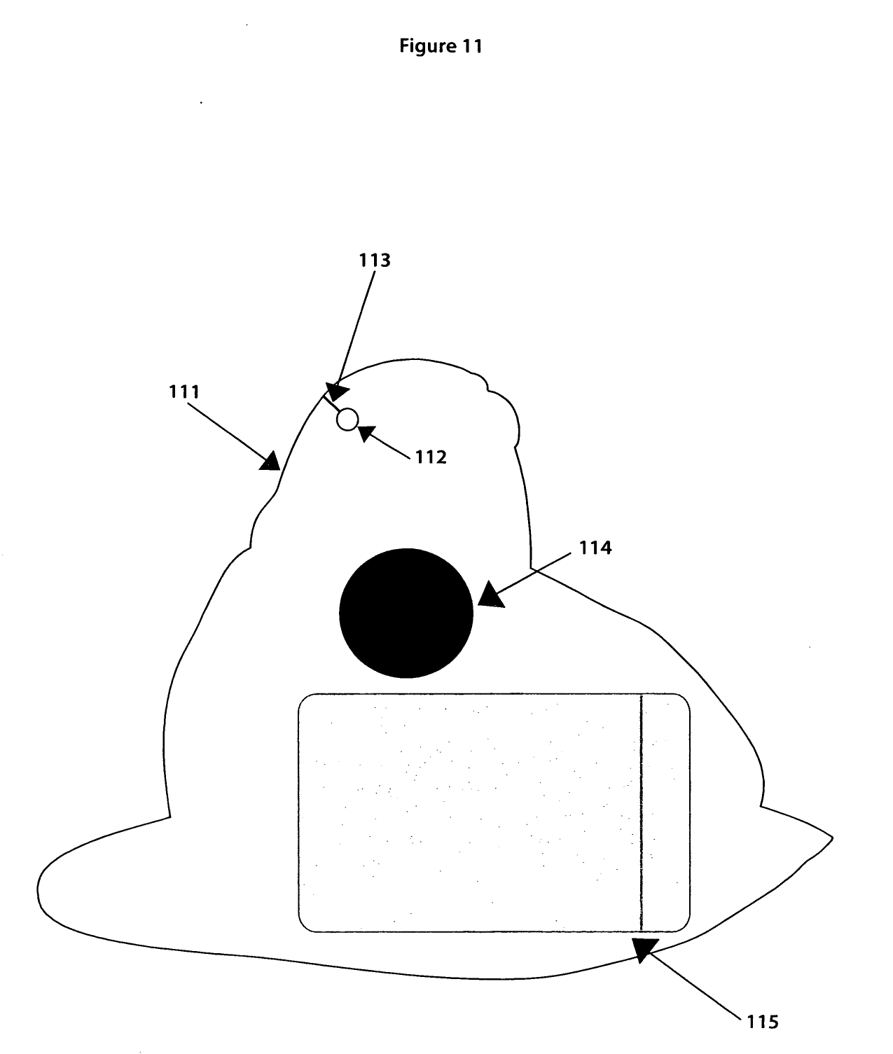

FIG. 12 is a back view of an eagles greeting card with poly pocket, magnet, and aperture with easy release mechanism used for hanging purposes and/or attaching a decorative item like a tassel.

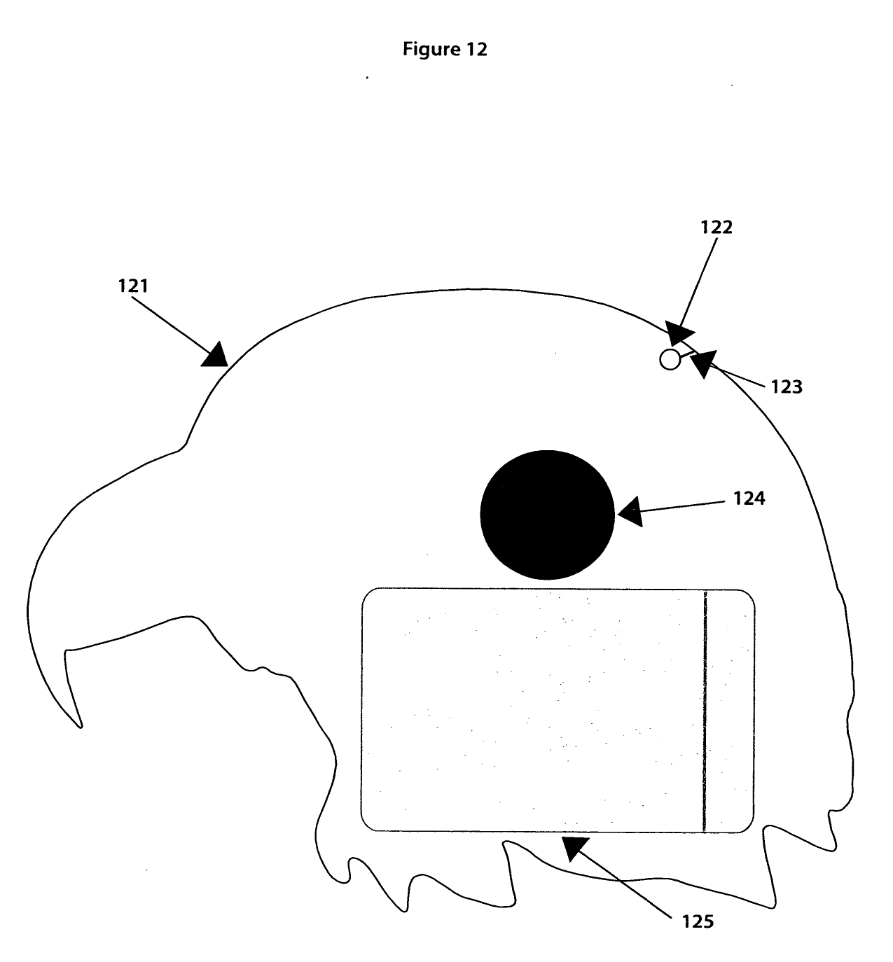

FIG. 13 is a back view of a bull dog greeting card with poly pocket, magnet, and aperture with easy release mechanism used for hanging purposes and/or attaching a decorative item like a tassel.

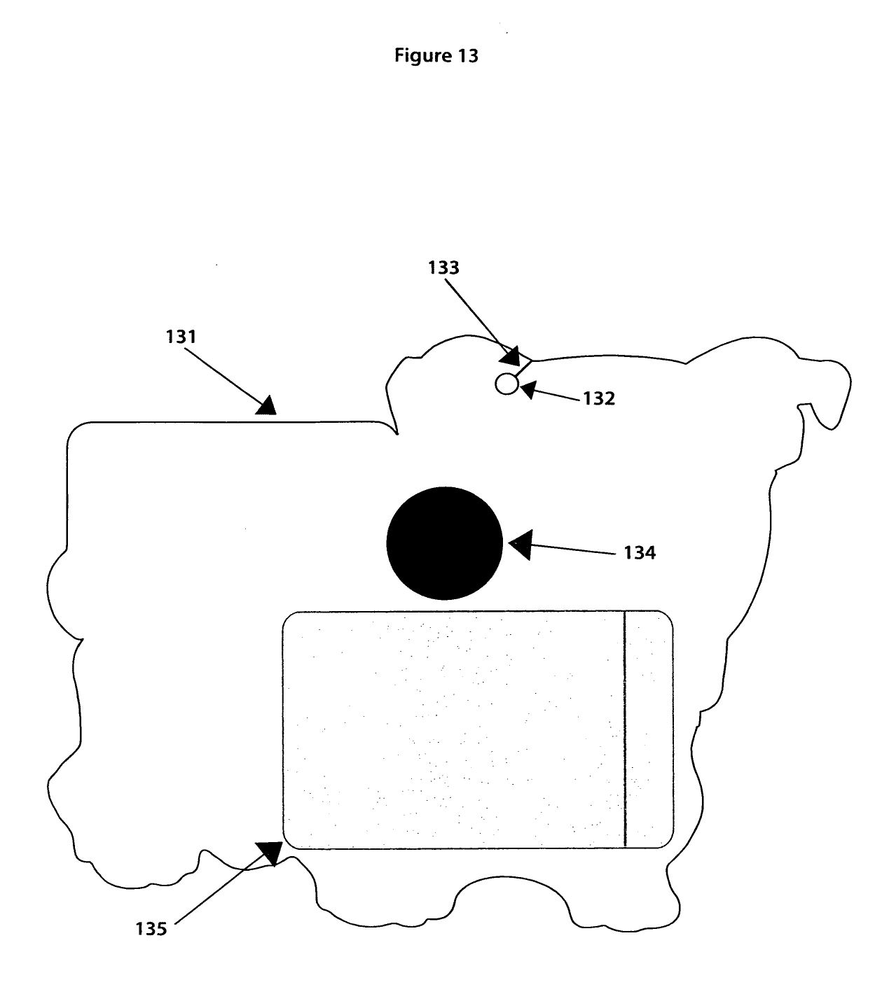

FIG. 14 is a back view of bear n'bees, her birthday wish, and beach-surfing greeting cards with poly pocket, magnet, and aperture with easy release mechanism used for hanging purposes and/or attaching a decorative item like a tassel.

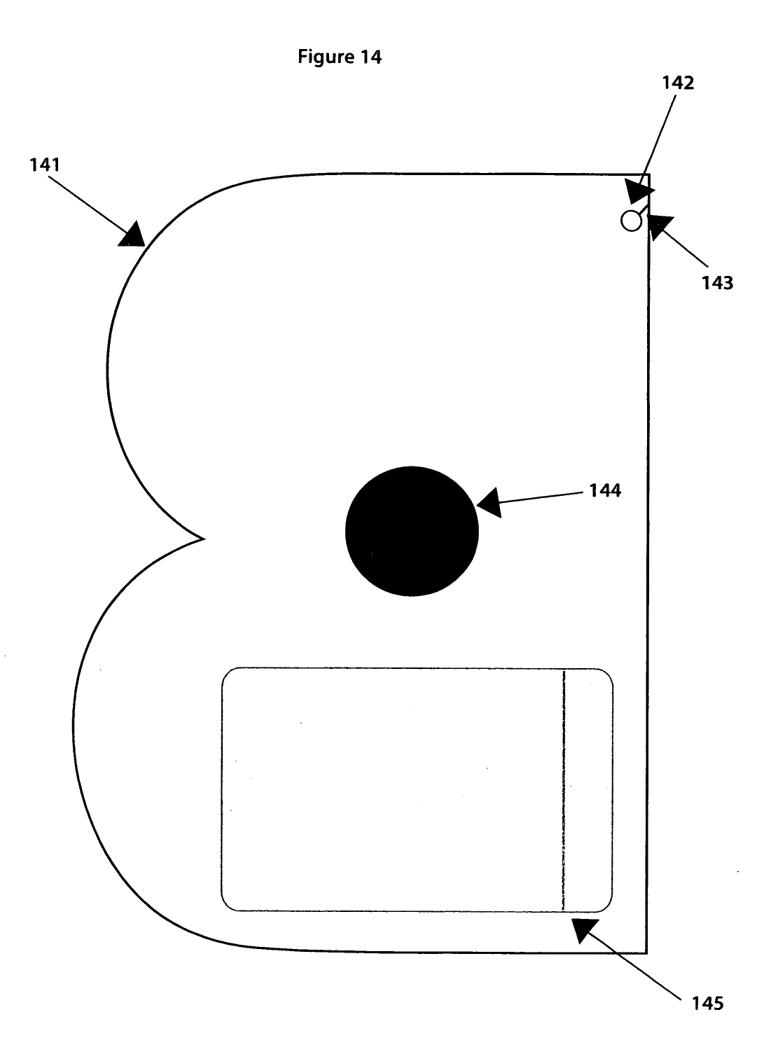

FIG. 15 is a back view cross bones greeting card with with poly pocket, magnet, and aperture with easy release mechanism used for hanging purposes and/or attaching a decorative item like a tassel.

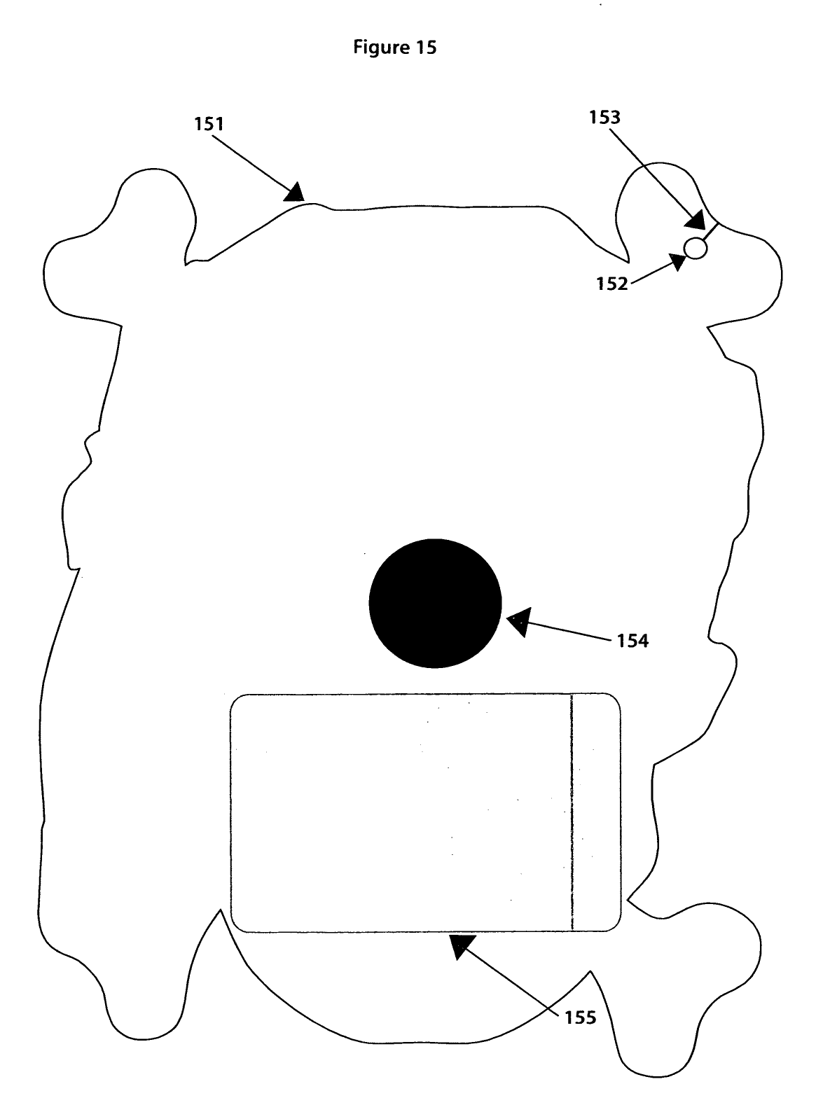

FIG. 16 is a back view of a biker dude greeting card with poly pocket, magnet, and aperture with easy release mechanism used for hanging purposes and/or attaching a decorative item like a tassel.

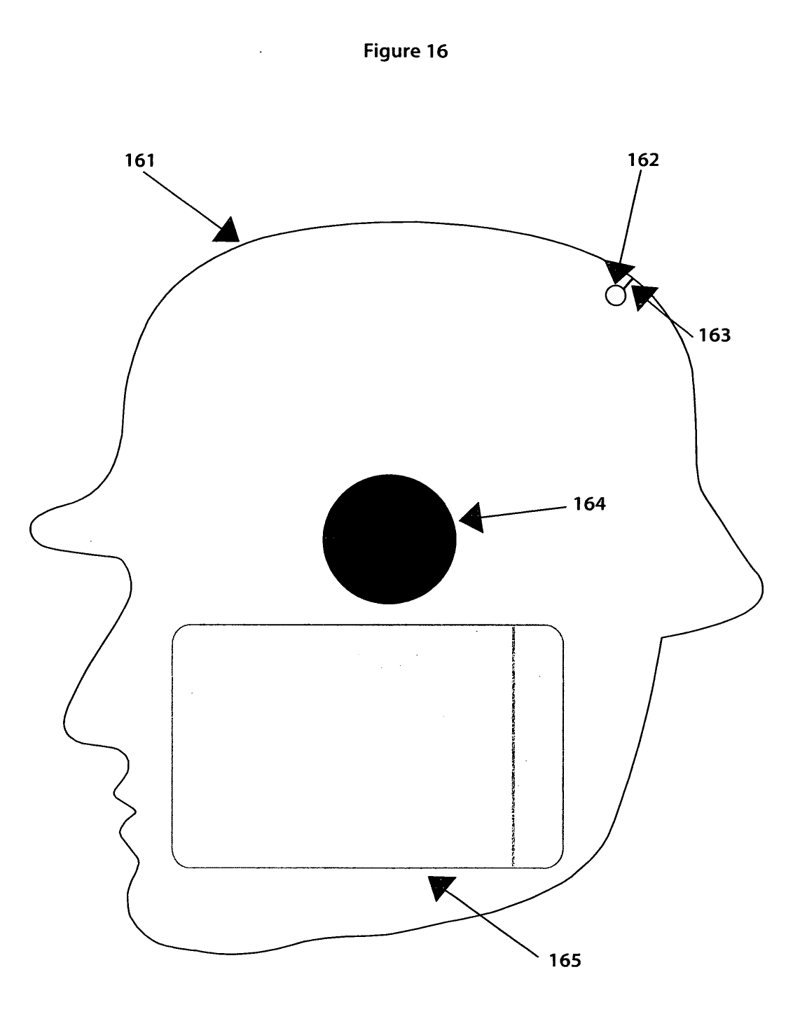

FIG. 17 is a back view of an Indian greeting card with poly pocket, magnet, and aperture with easy release mechanism used for hanging purposes and/or attaching a decorative item like a tassel.

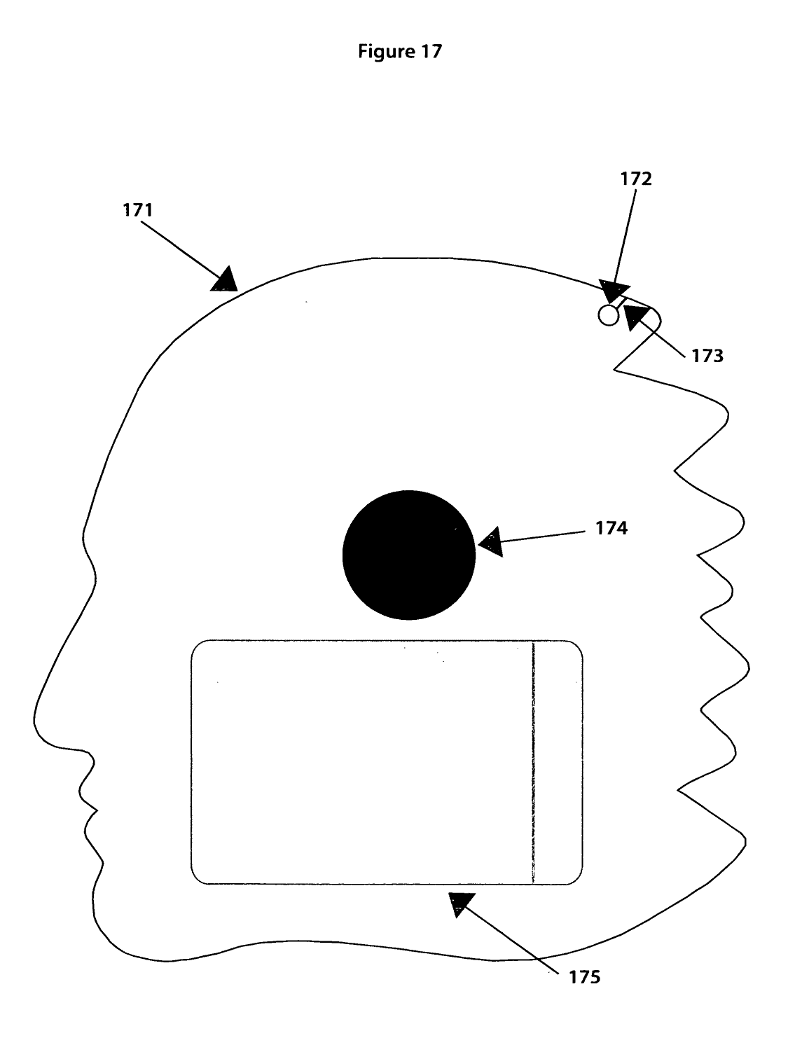

FIG. 18 is a back view of a yellow jacket greeting card with poly pocket, magnet, and aperture with easy release mechanism used for hanging purposes and/or attaching a decorative item like a tassel.

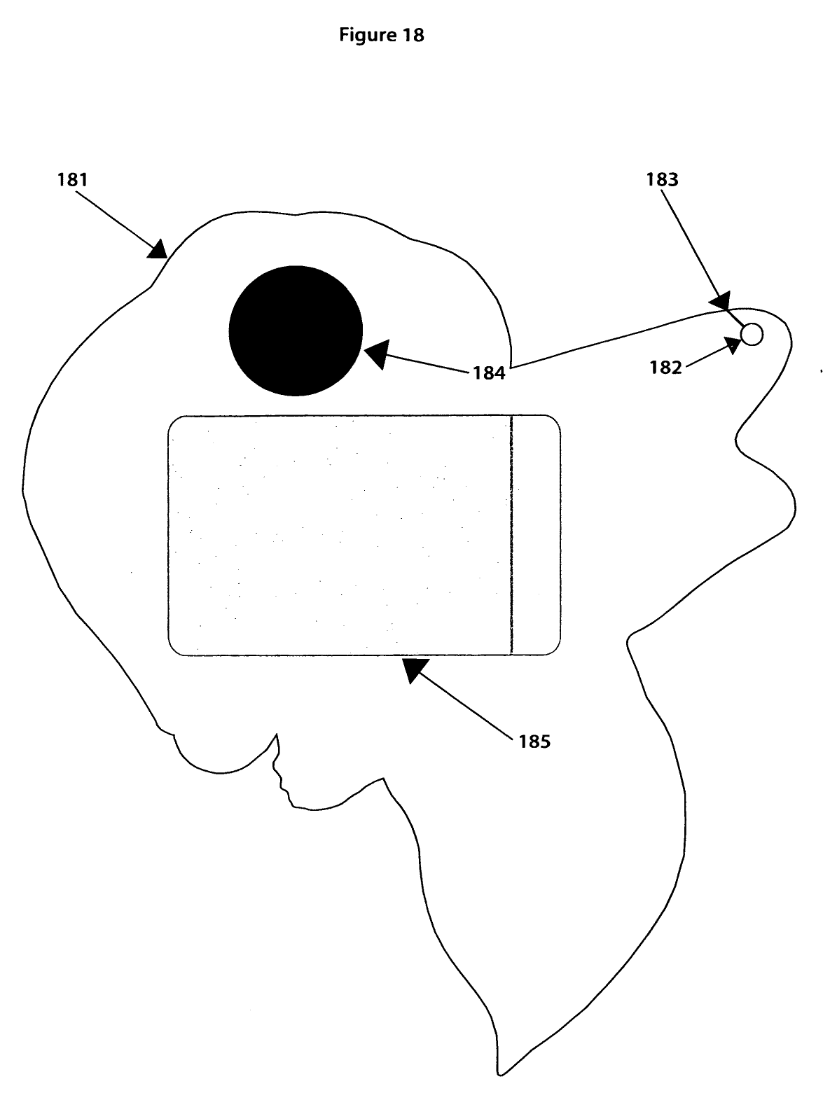

FIG. 19 is a back view of a Olympic sports greeting cards with poly pocket, magnet, and aperture with easy release mechanism used for hanging purposes and/or attaching a decorative item like a tassel.

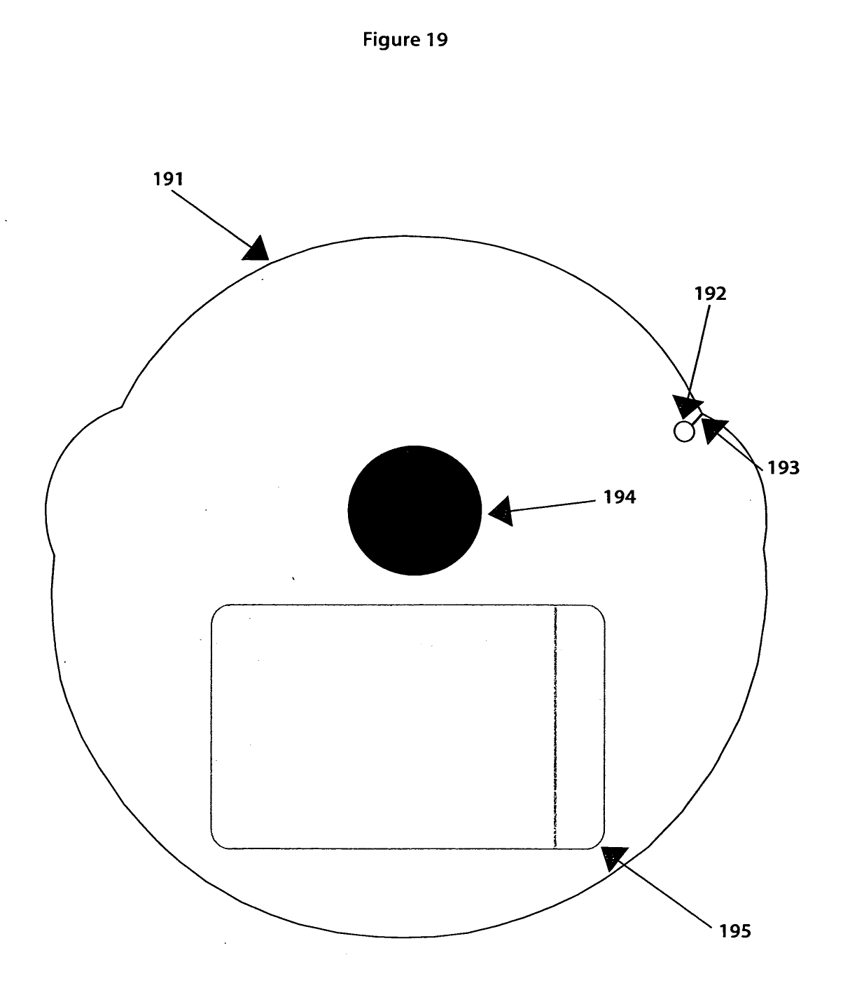

FIG. 20 is a back view of a orange and white tiger greeting cards with poly pocket, magnet, and aperture with easy release mechanism used for hanging purposes and/or attaching a decorative item like a tassel.

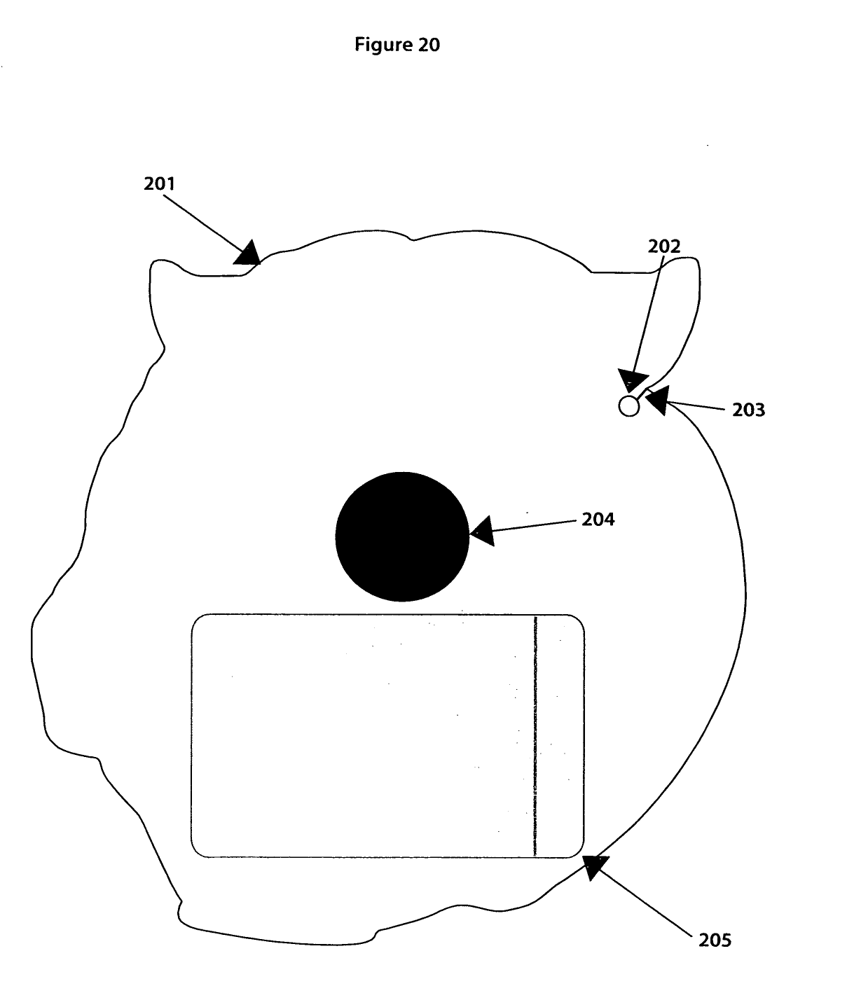

FIG. 21 is a back view of a lion greeting card with poly pocket, magnet, and aperture with easy release mechanism used for hanging purposes and/or attaching a decorative item like a tassel.

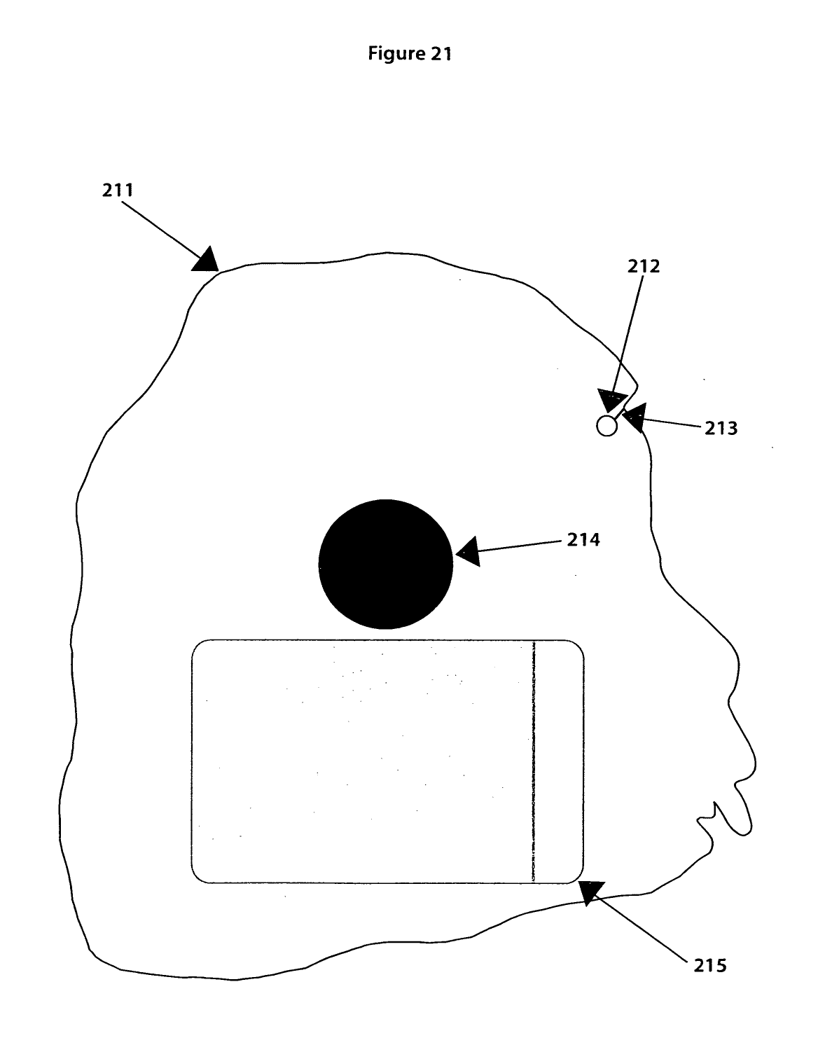

FIG. 22 is a back view of a pirate greeting card with poly pocket, magnet, and aperture with easy release mechanism used for hanging purposes and/or attaching a decorative item like a tassel.

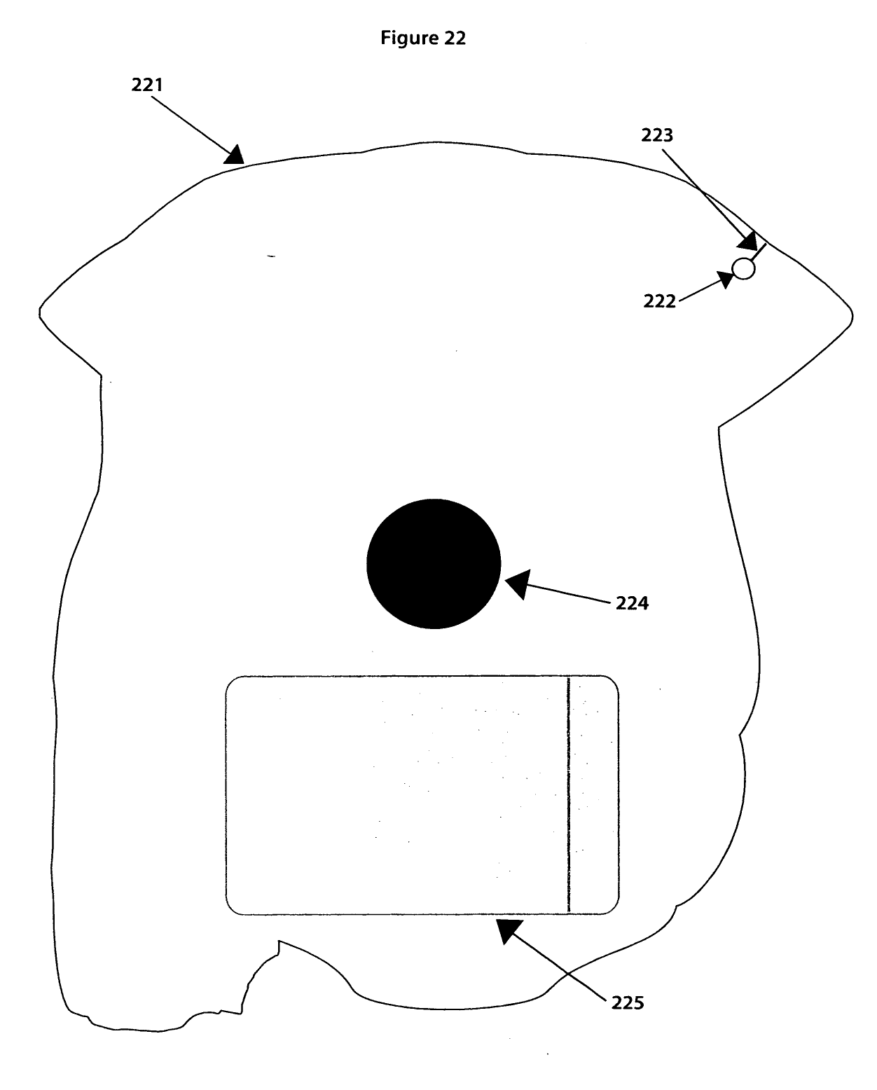

FIG. 23 is a back view of a space shuttle greeting card with poly pocket, magnet, and aperture with easy release mechanism used for hanging purposes and/or attaching a decorative item like a tassel.

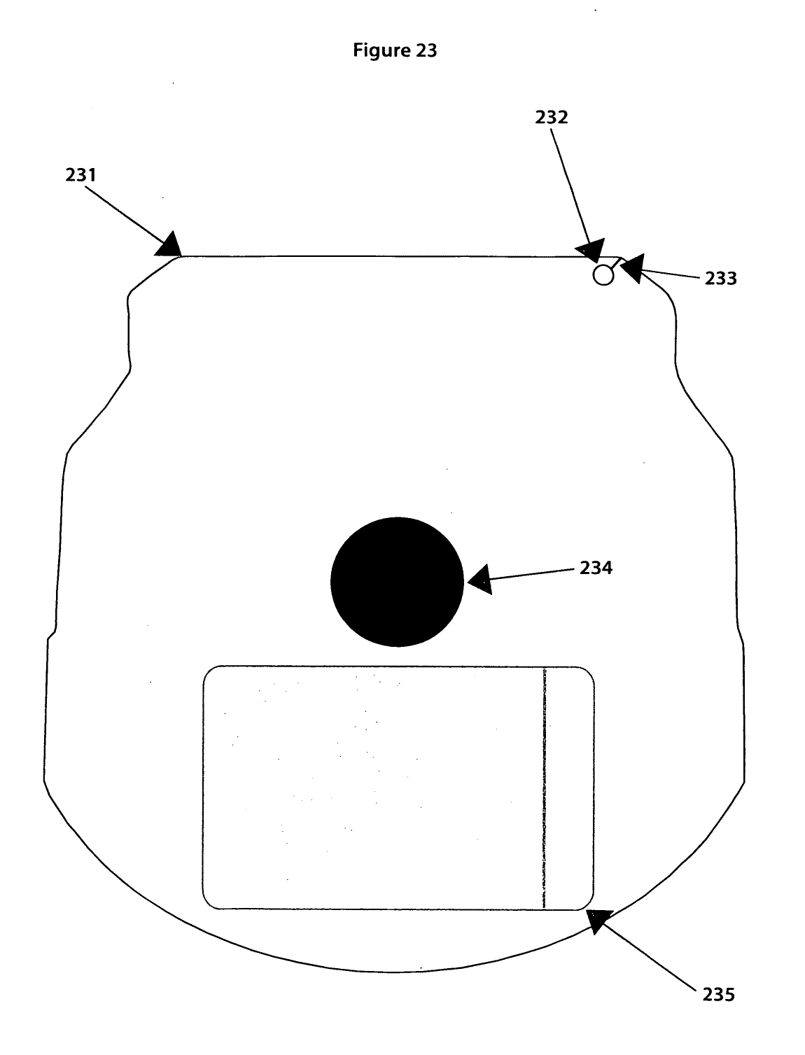

FIG. 24 is a back view of a derby girl greeting card with poly pocket, magnet, and aperture with easy release mechanism used for hanging purposes and/or attaching a decorative item like a tassel.

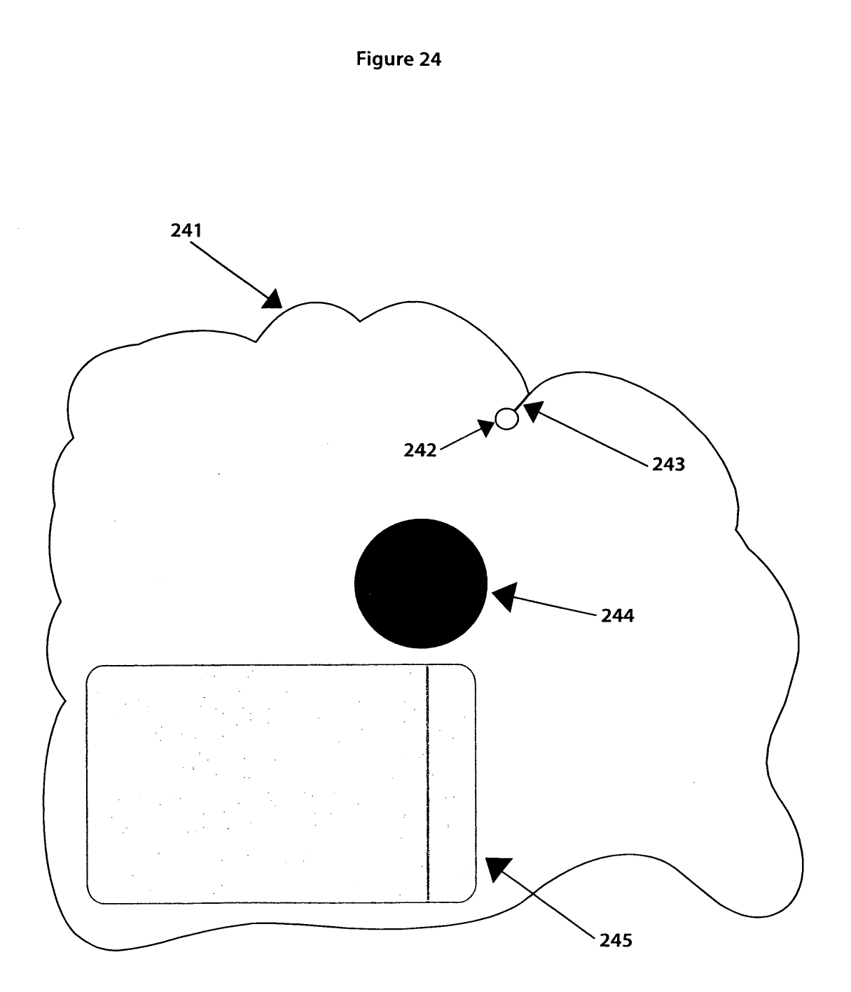

FIG. 25 is a back view of a santa claus, balloon flight, zombie, and UN soldier greeting greeting cards with poly pocket, magnet, and aperture with easy release mechanism used for hanging purposes and/or attaching a decorative item like a tassel.

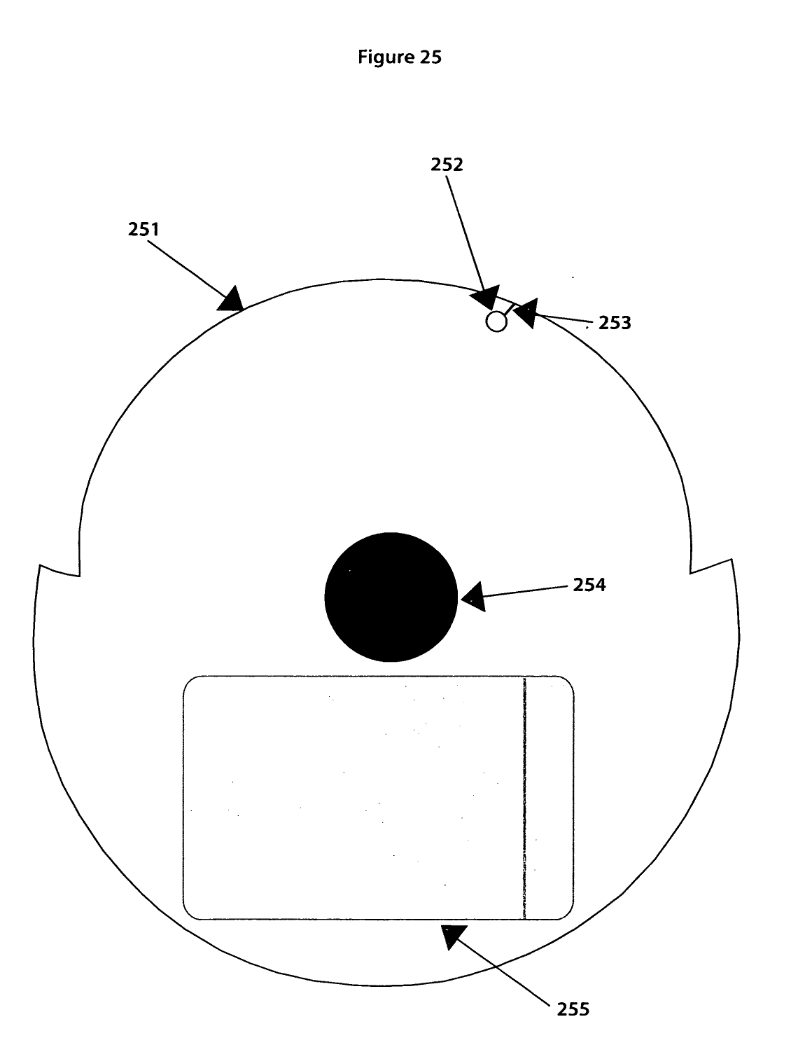

FIG. 26 is a back view of a “birthday-alert”greeting card with poly pocket, magnet, and aperture with easy release mechanism used for hanging purposes and/or attaching a decorative item like a tassel.

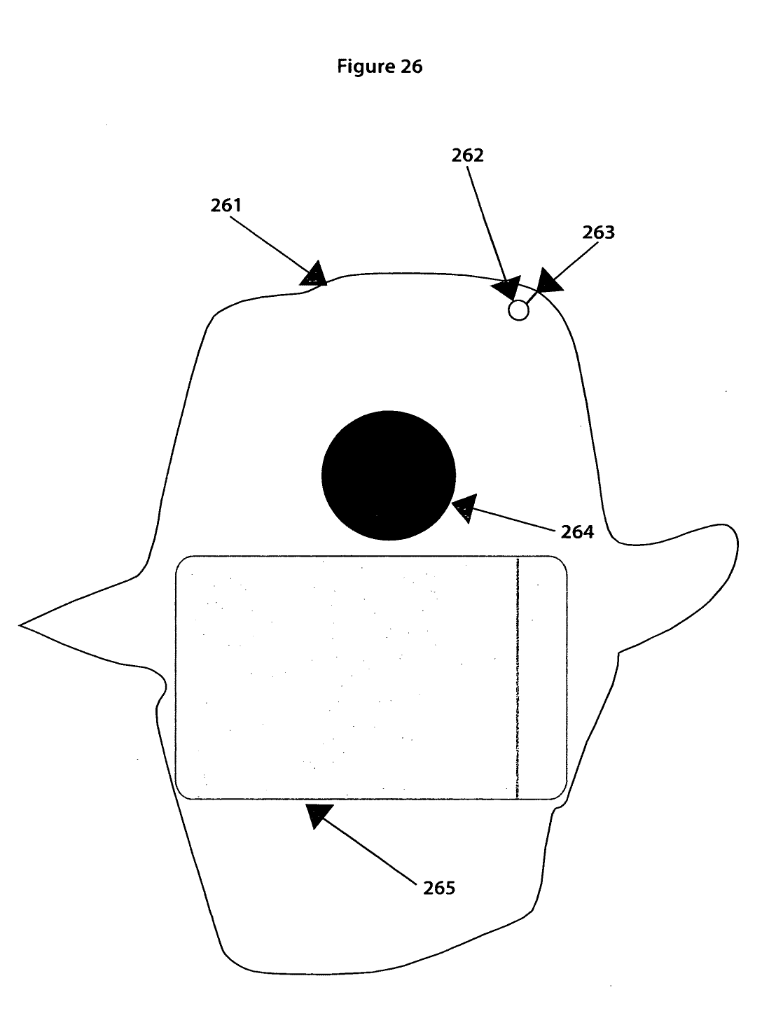

FIG. 27 is a back view of a bull greeting card wth poly pocket, magnet, and aperture with easy release mechanism used for hanging purposes and/or attaching a decorative item like a tassel.

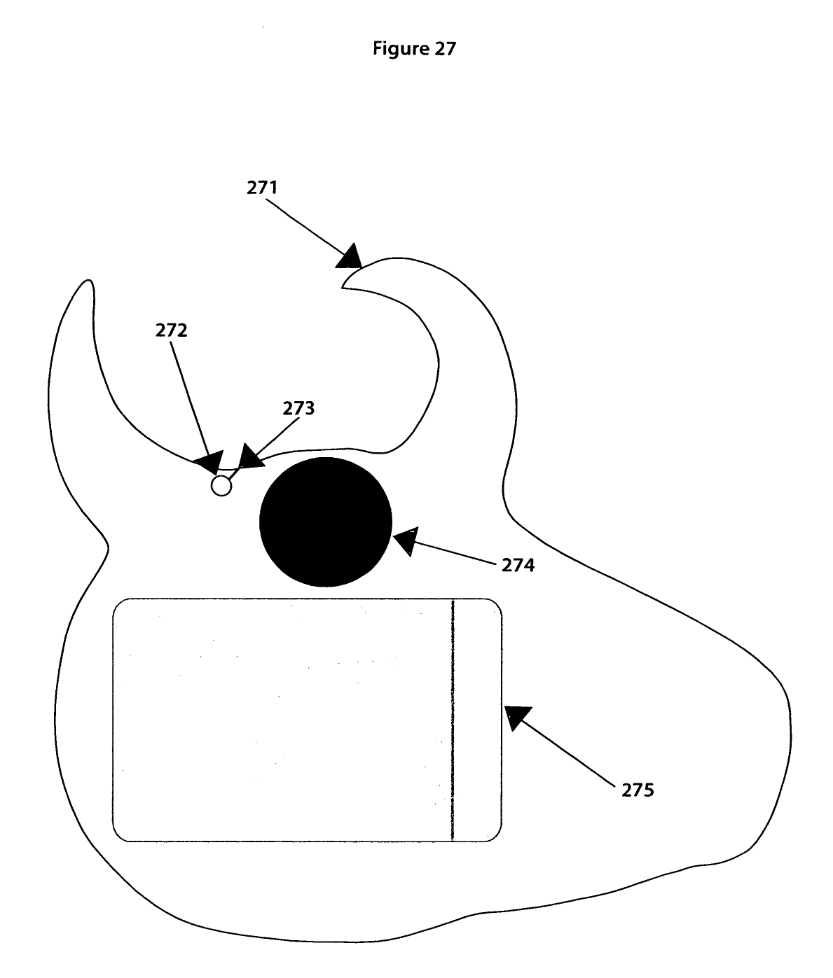

FIG. 28 is a back view of a bunnies magic greeting card with poly pocket, magnet, and aperture with easy release mechanism used for hanging purposes and/or attaching a decorative item like a tassel.

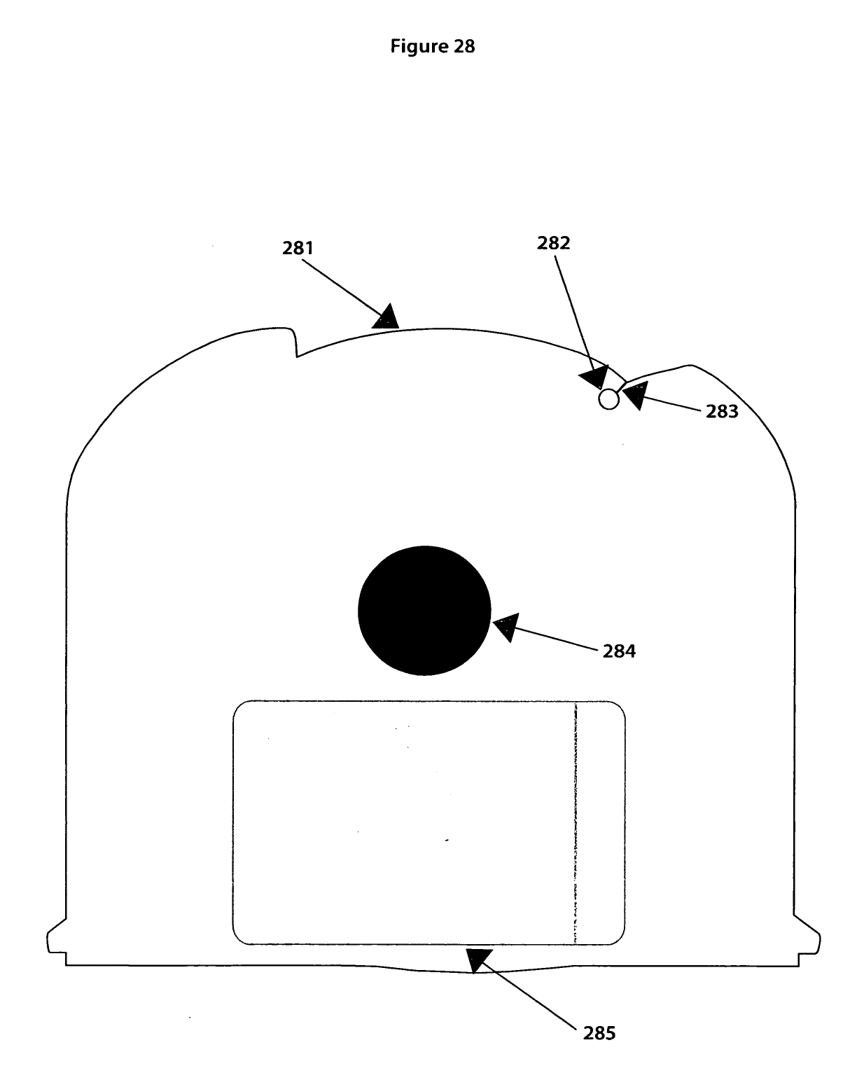

FIG. 29 is a back view of a whales and white water rafting greeting card with poly pocket, magnet, and aperture with easy release mechanism used for hanging purposes and/or attaching a decorative item like a tassel.

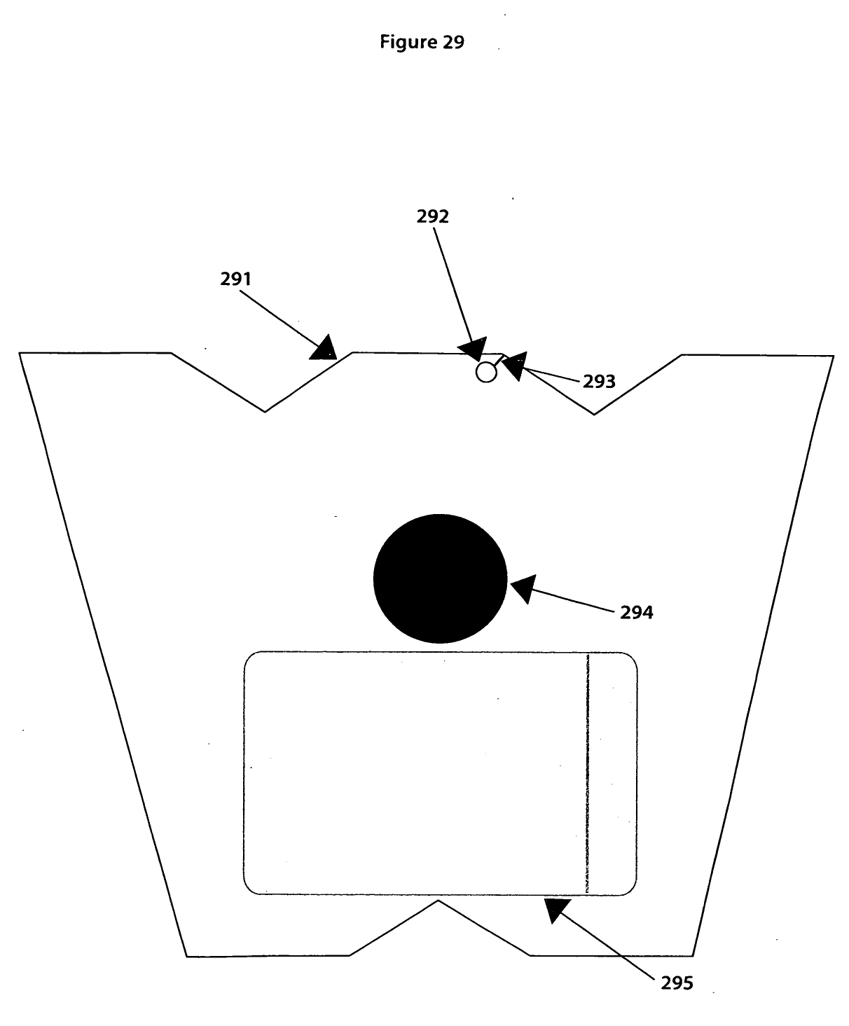

FIG. 30 is a back view of a cowboy and country singer greeting cards with poly card pocket, magnet, and aperture with easy release mechanism used for hanging purposes and/or attaching a decorative item like a tassel.

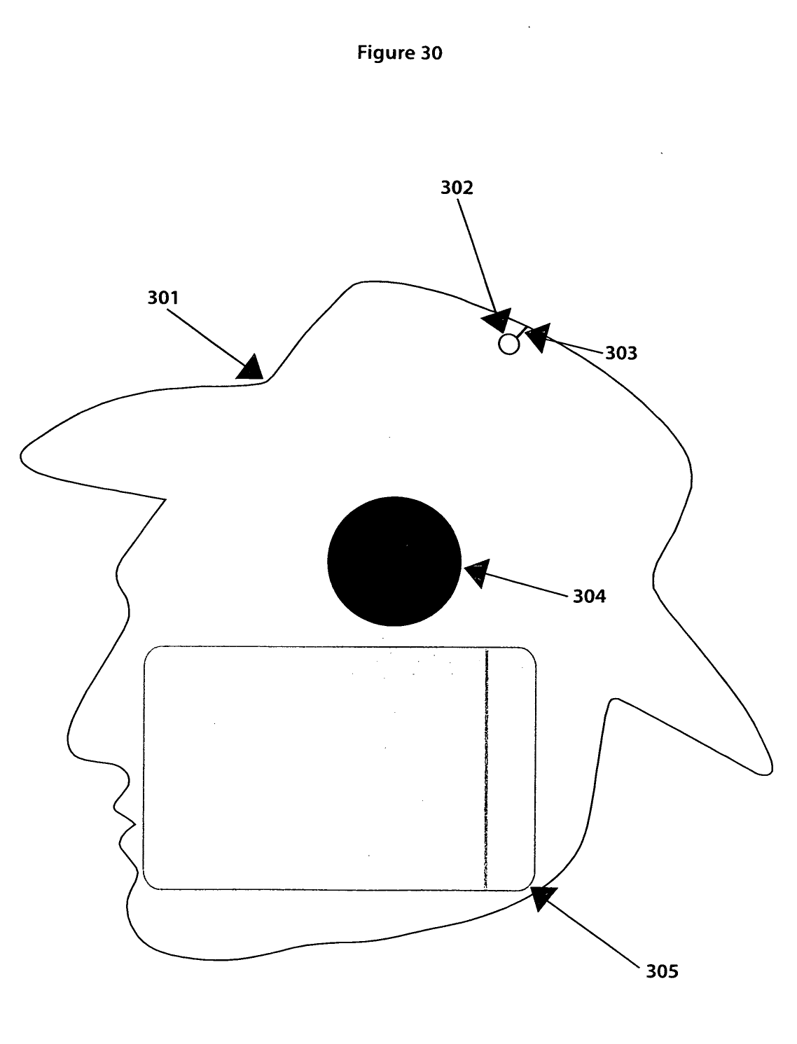

FIG. 31 is back view of a Catalina diver, cardinal direction, Little Mermaid, camping, Communion & Confirmation, bring in the New Year, Christmas carols, Three Wise Men greeting cards with poly pocket, magnet, and aperture with easy release mechanism used for hanging purposes and/or attaching a decorative item like a tassel.

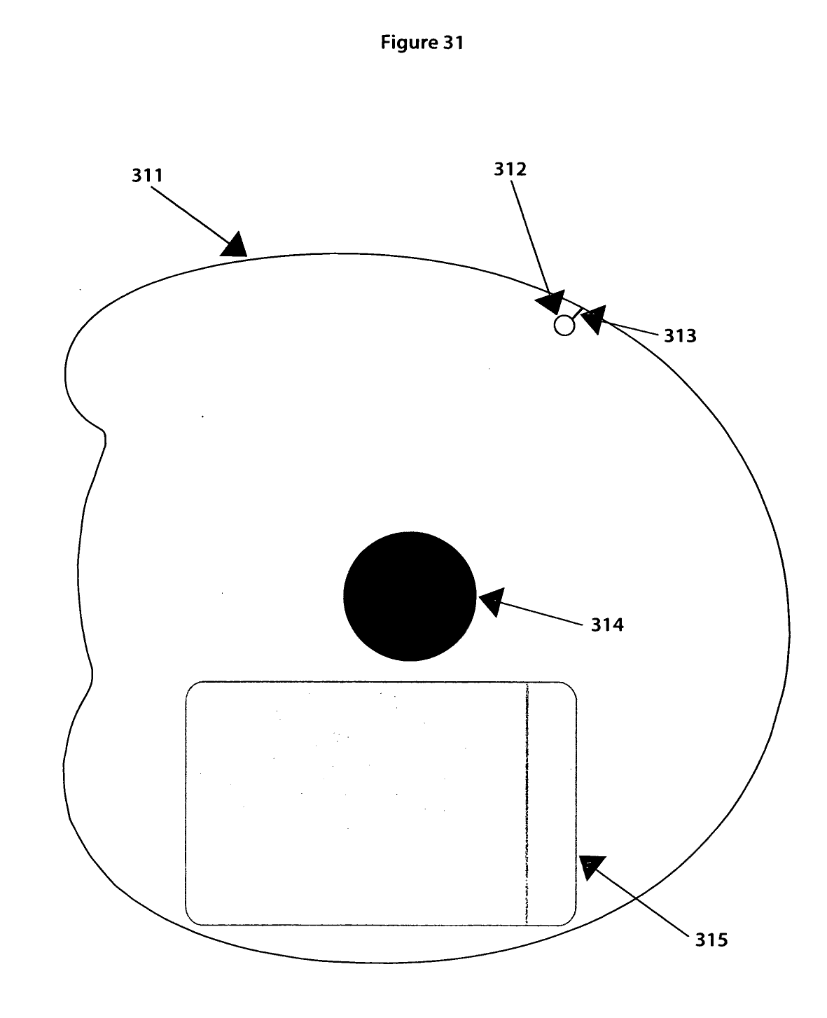

FIG. 32 is back view of a ducks greeting card with poly pocket, magnet, and aperture with easy release mechanism used for hanging purposes and/or attaching a decorative item like a tassel.

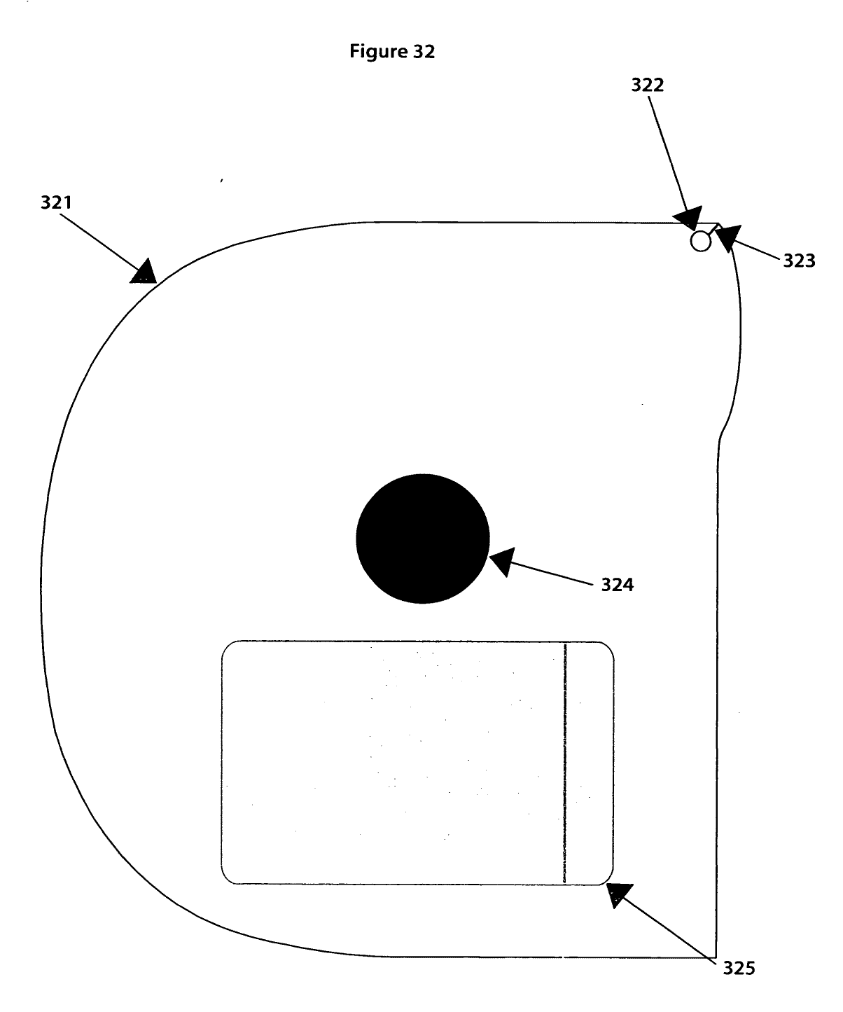

FIG. 33 is a back view of a computer mouse greeting card with poly pocket, magnet, and aperture with easy release mechanism used for hanging purposes and/or attaching a decorative item like a tassel.

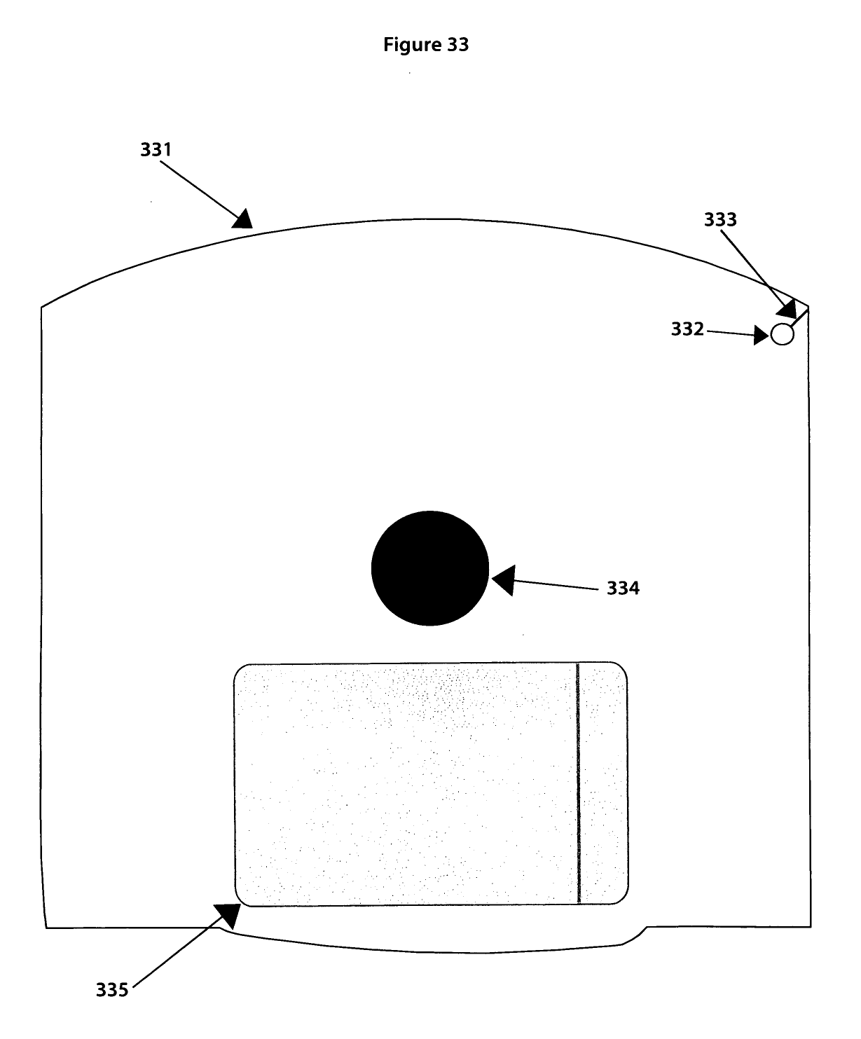

FIG. 34 is a back view of a Air Force pilot greeting card with poly pocket, magnet, and aperture with easy release mechanism used for hanging purposes and/or attaching a decorative item like a tassel.

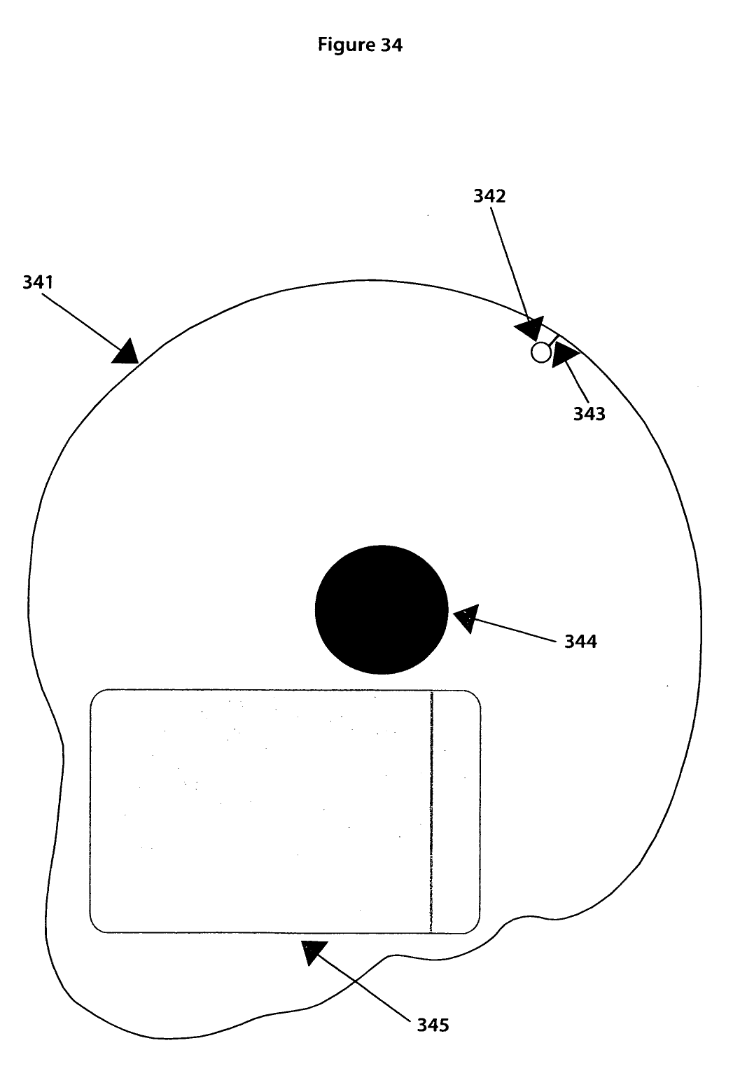

FIG. 35 is a back view of a navy captain-train conductor-airline pilot-submarine commander, WWII marine and Noah greeting cards with poly pocket, magnet, and aperture with easy release mechanism used for hanging purposes and/or attaching a decorative item like a tassel.

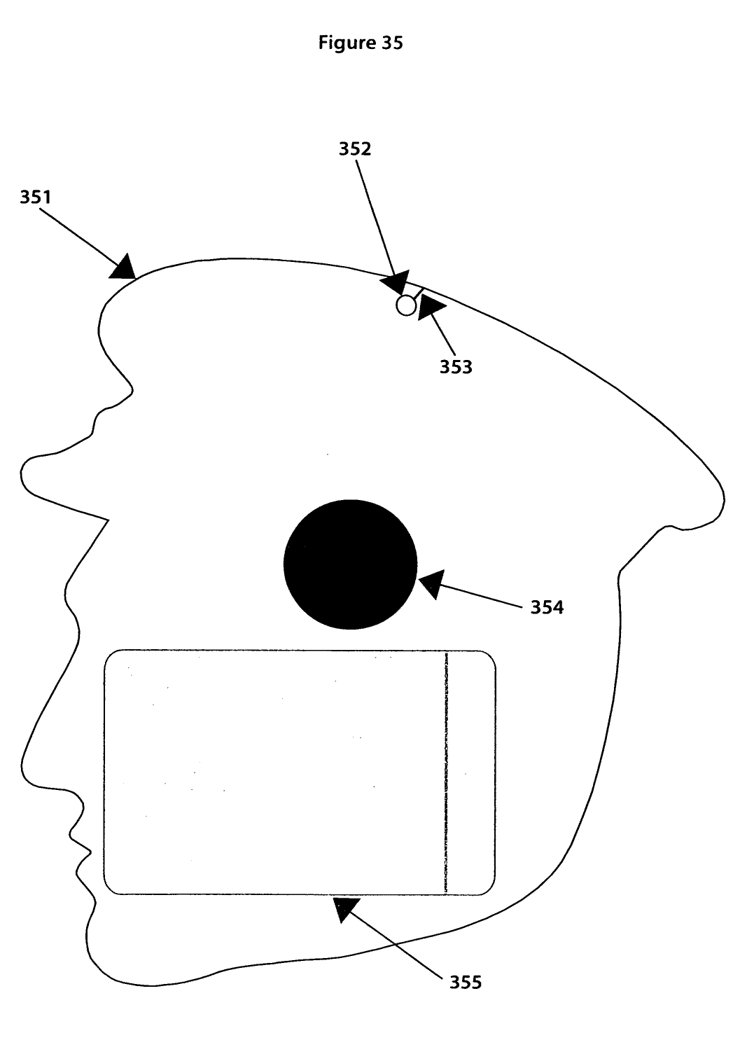

FIG. 36 is back view of a peace symbol greeting card with poly pocket, magnet, and aperture with easy release mechanism used for hanging purposes and/or attaching a decorative item like a tassel.

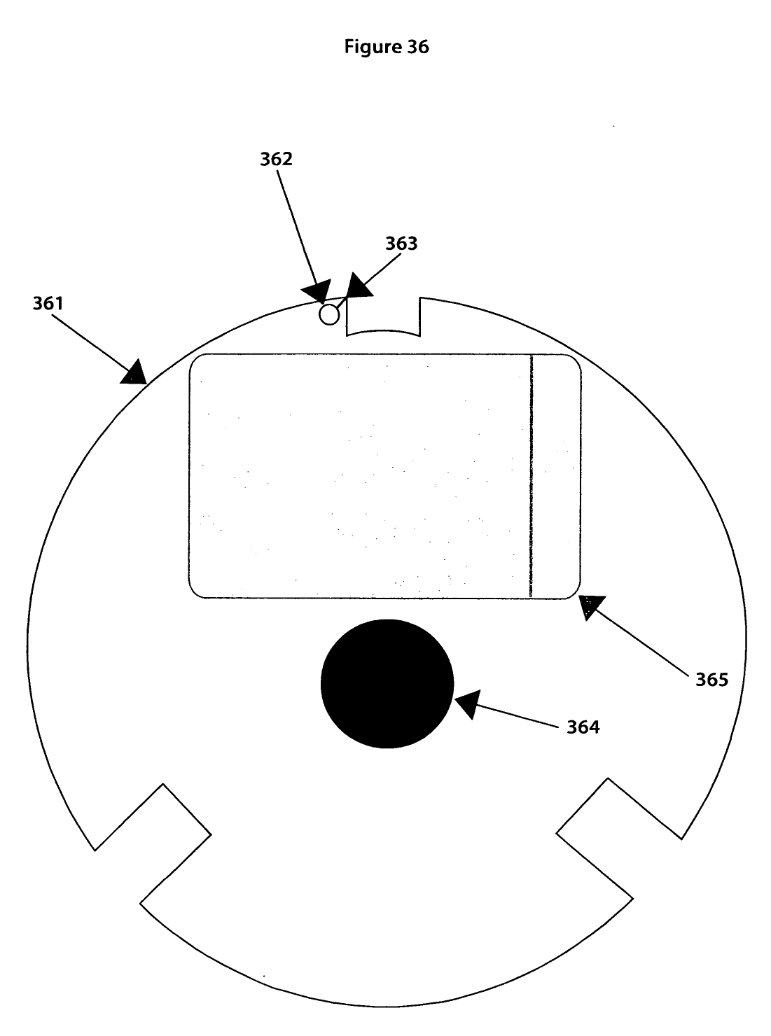

FIG. 37 is back view of a elephant greeting card with poly pocket, magnet, and aperture with easy release mechanism used for hanging purposes and/or attaching a decorative item like a tassel.

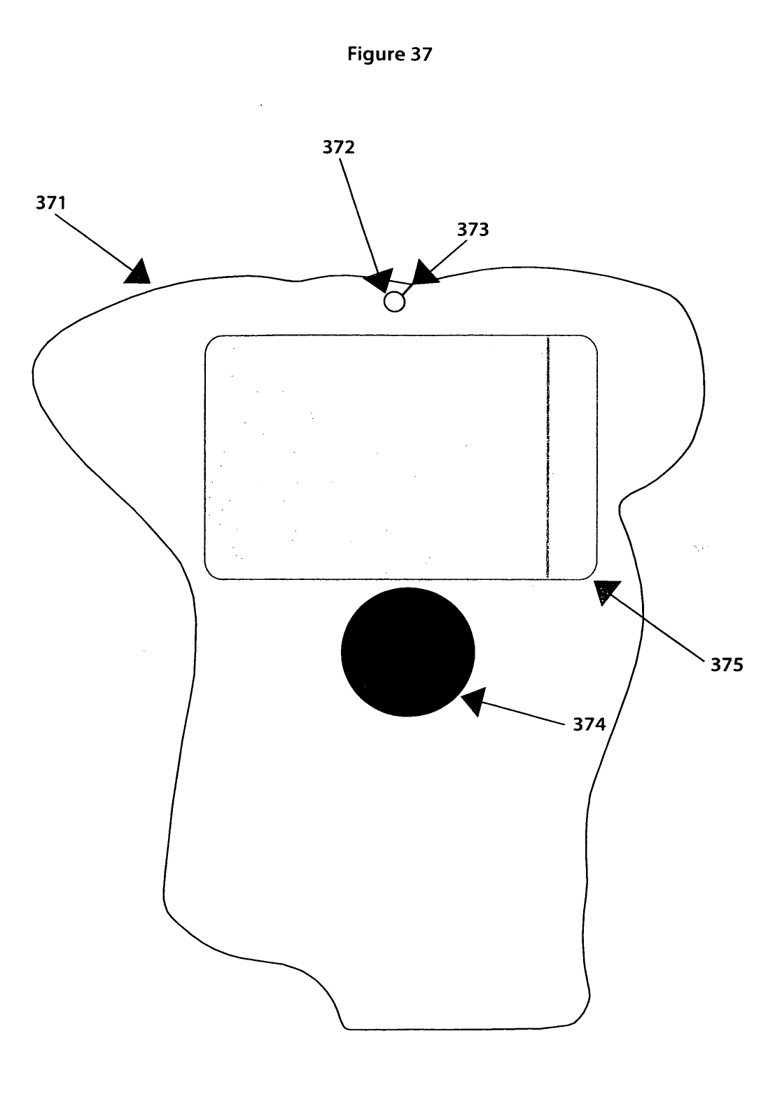

FIG. 38 is a back view of a baseball player and golfer greeting cards with poly pocket, magnet, and aperture with easy release mechanism used for hanging purposes and/or attaching a decorative item like a tassel.

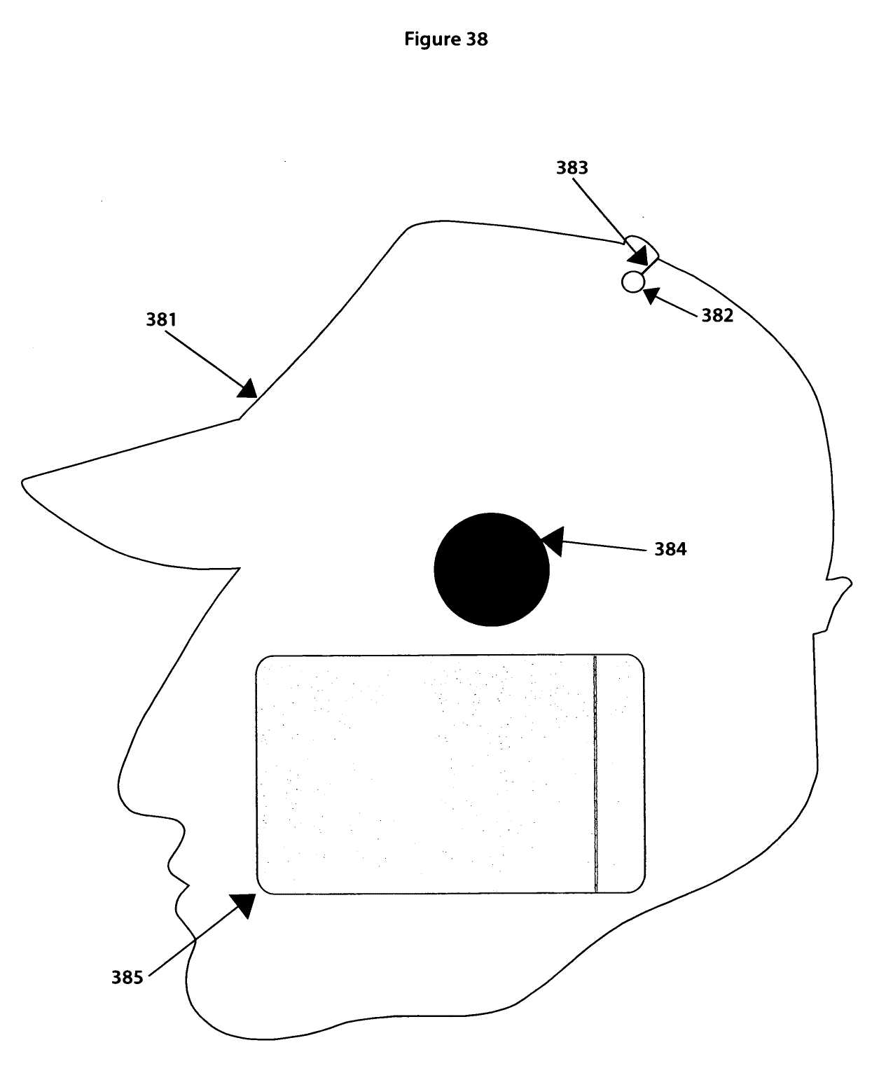

FIG. 39 is a back view of a football player greeting card with poly pocket, magnet, and aperture with easy release mechanism used for hanging purposes and/or attaching a decorative item like a tassel.

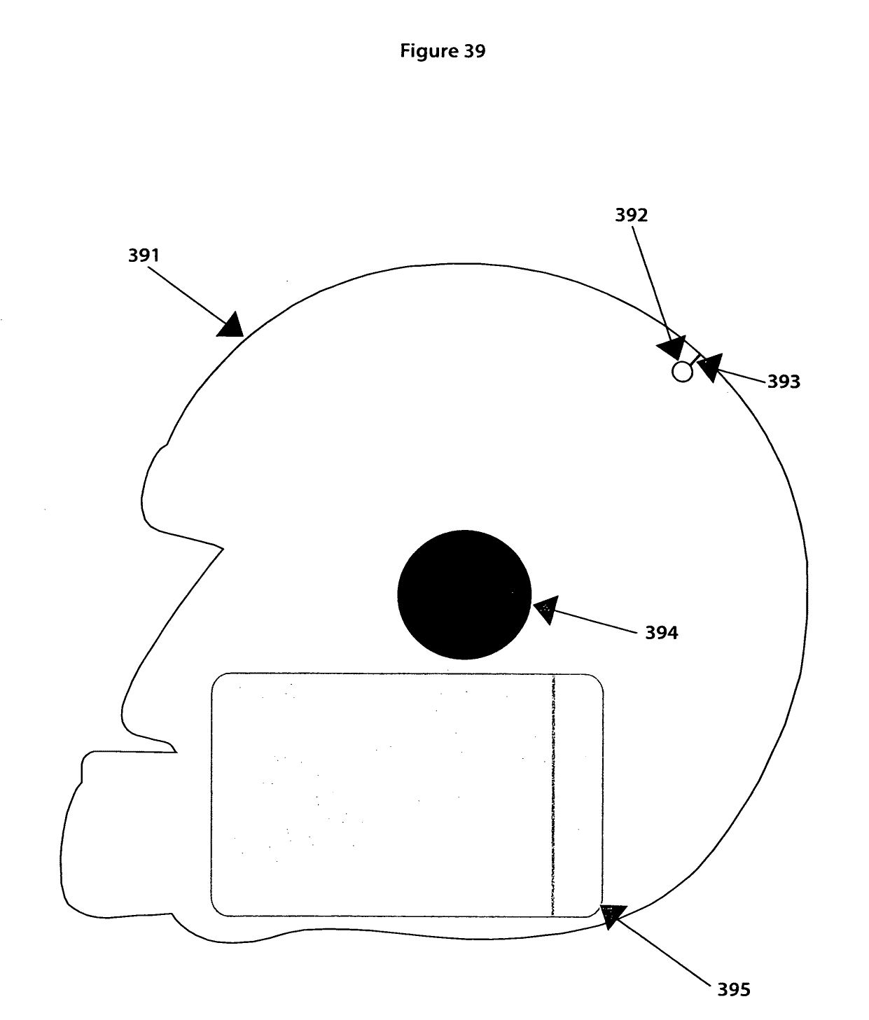

FIG. 40 is a back view of a ram greeting card with poly pocket, magnet, and aperture with easy release mechanism used for hanging purposes and/or attaching a decorative item like a tassel.

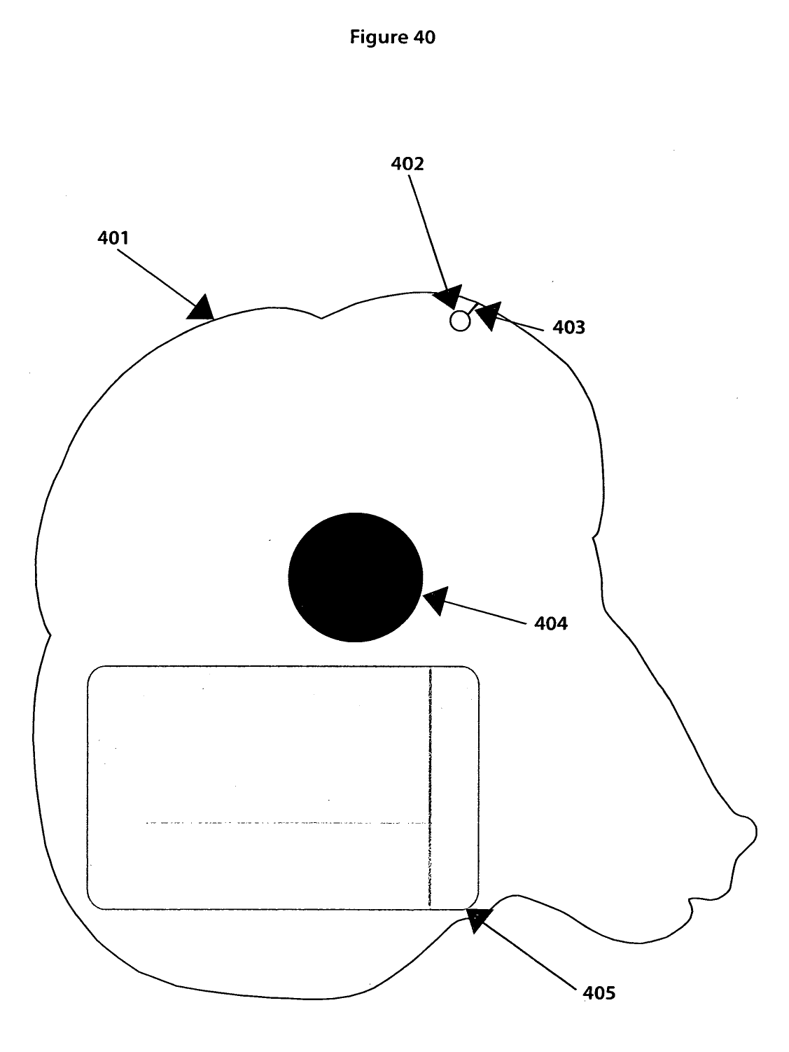

FIG. 41 is a back view of a road runner racer greeting card withpoly pocket, magnet, and aperture with easy release mechanism used for hanging purposes and/or attaching a decorative item like a tassel.

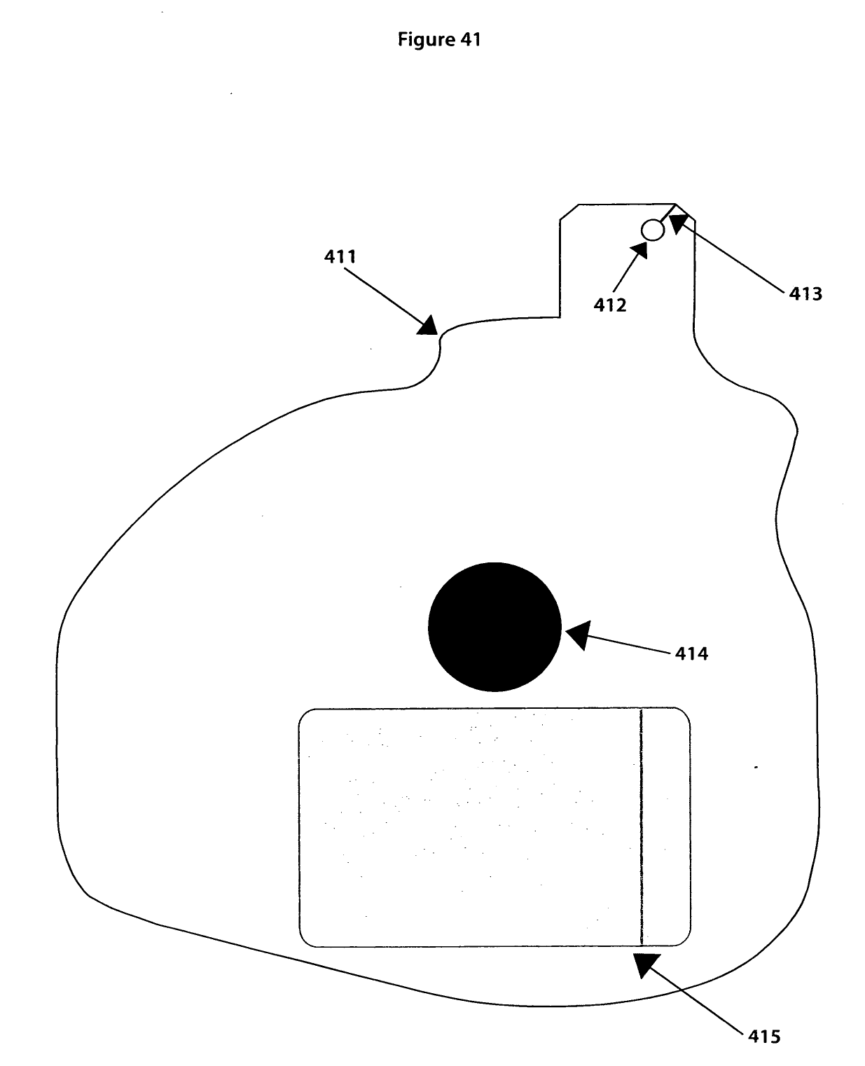

FIG. 42 is a back view of a dinosaurs, travels (down-under), fathers day, “his”get well, “her”get well, his educational dreams greeting cards with poly pocket, magnet, and aperture with easy release mechanism used for hanging purposes and/or attaching a decorative item like a tassel.

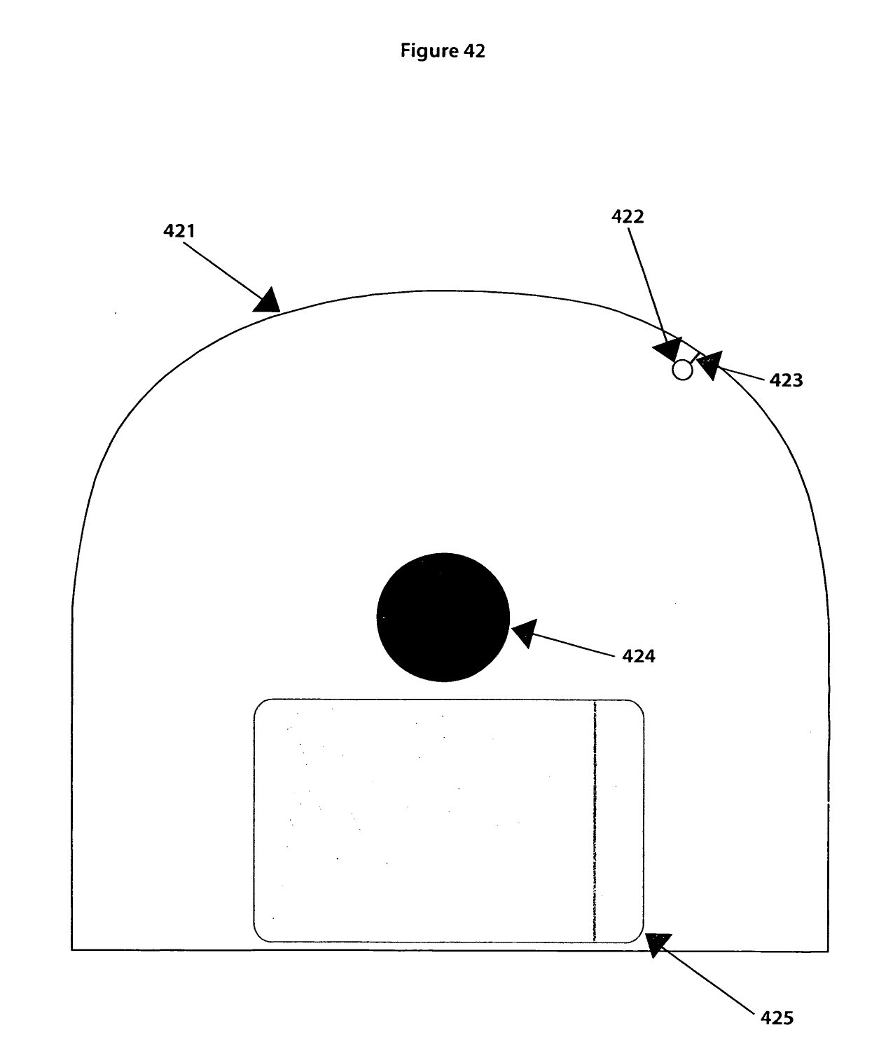

FIG. 43 is back view of a sympathy (Christian) greeting card with poly pocket, magnet, and aperture with easy release mechanism used for hanging purposes and/or attaching a decorative item like a tassel.

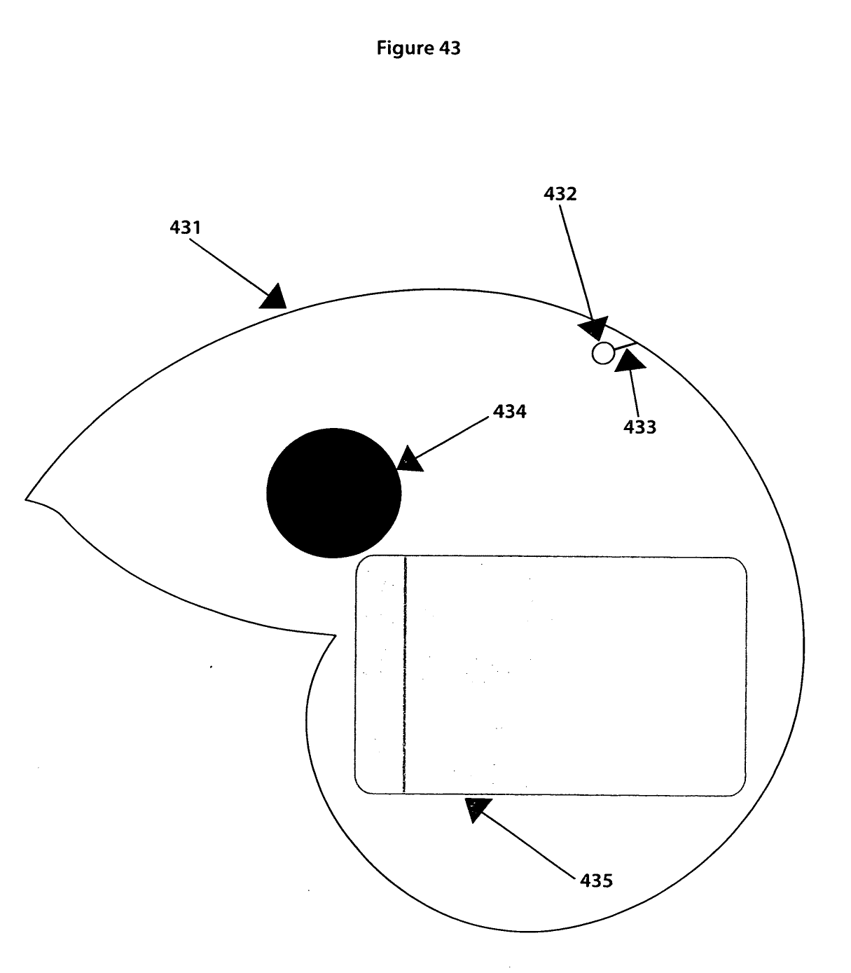

FIG. 44 is a back view of a dolphin greeting card with poly pocket, magnet, and aperture with easy release mechanism used for hanging purposes and/or attaching a decorative item like a tassel.

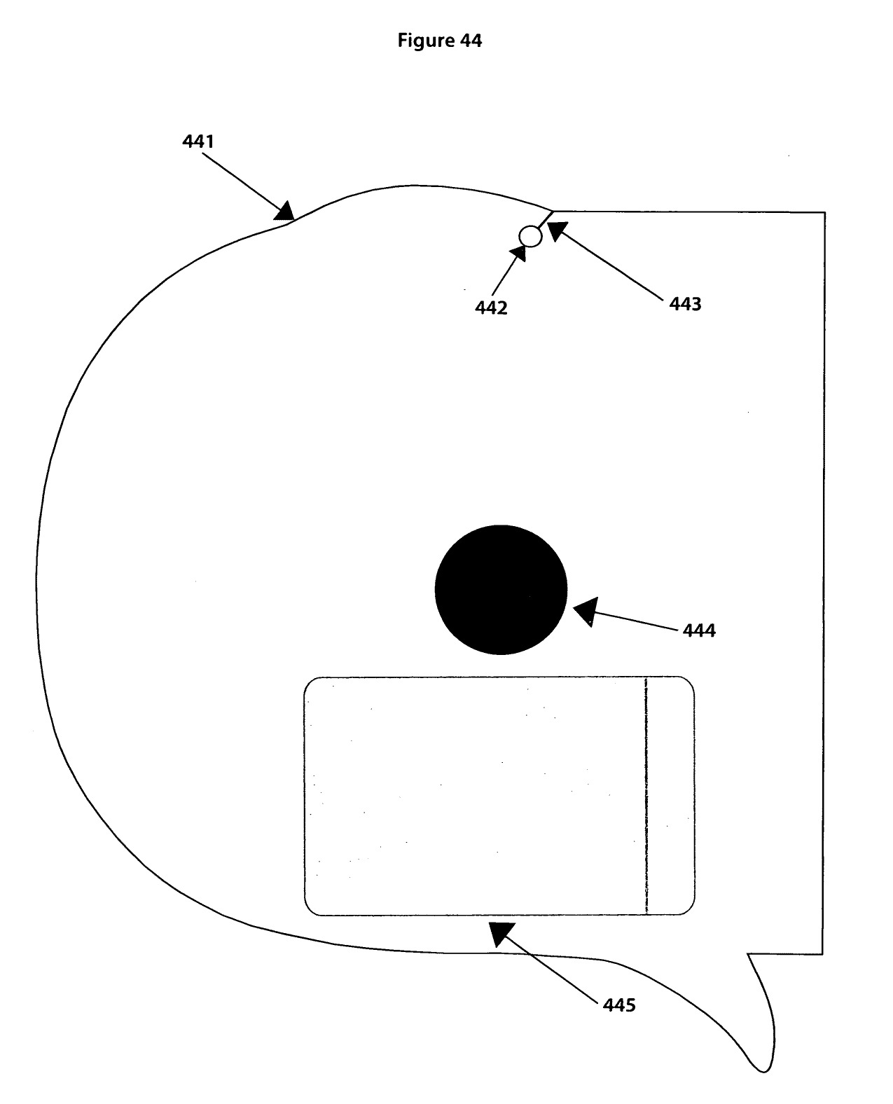

FIG. 45 is a back view of an owl greeting card with poly pocket, magnet, and aperture with easy release mechanism used for hanging purposes and/or attaching a decorative item like a tassel.

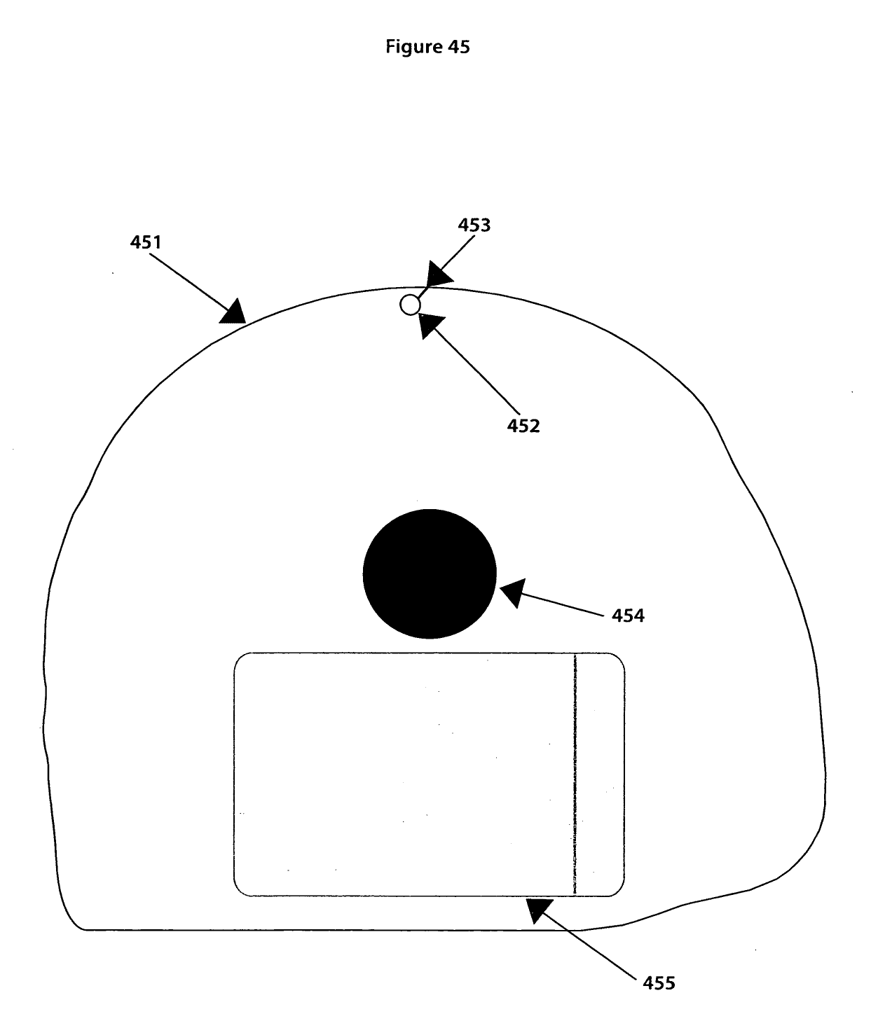

FIG. 46 is a back view of a piggy bankers greeting card with poly pocket, magnet, and aperture with easy release mechanism used for hanging purposes and/or attaching a decorative item like a tassel.

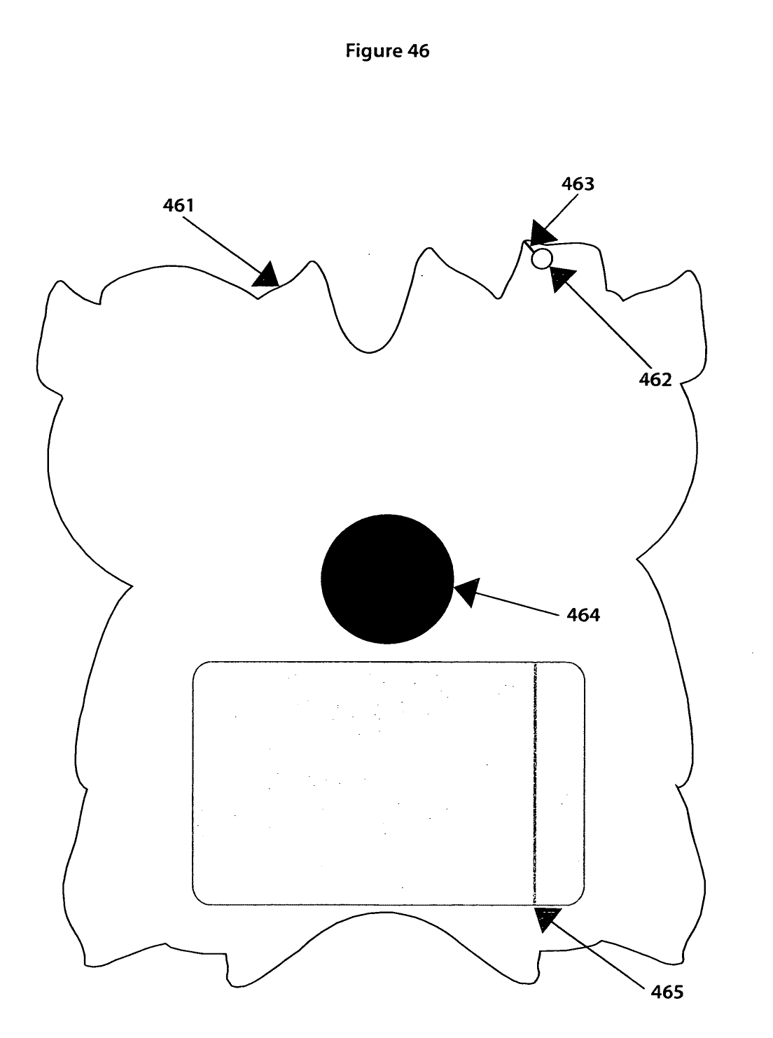

FIG. 47 is a back view of a fish bowl diver greeting card with poly pocket, magnet, and aperture with easy release mechanism used for hanging purposes and/or attaching a decorative item like a tassel.

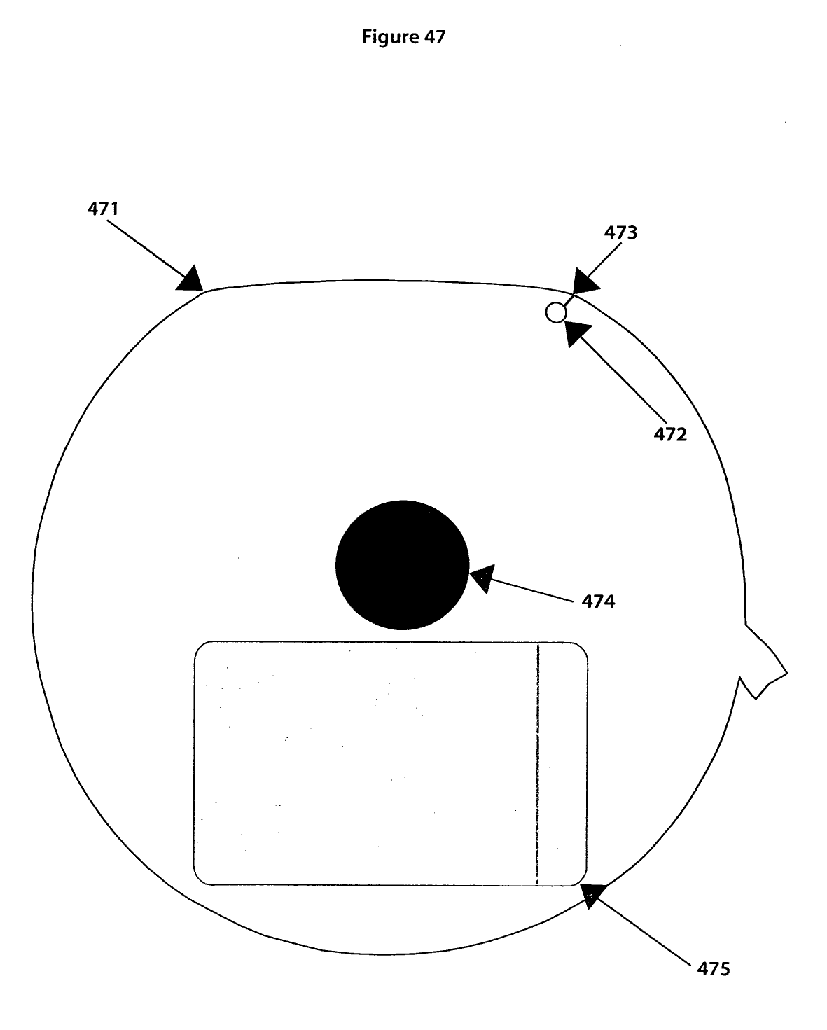

FIG. 48 is a back view of a Egyptian Pyramids greeting card with poly pocket, magnet, and aperture with easy release mechanism used for hanging purposes and/or attaching a decorative item like a tassel.

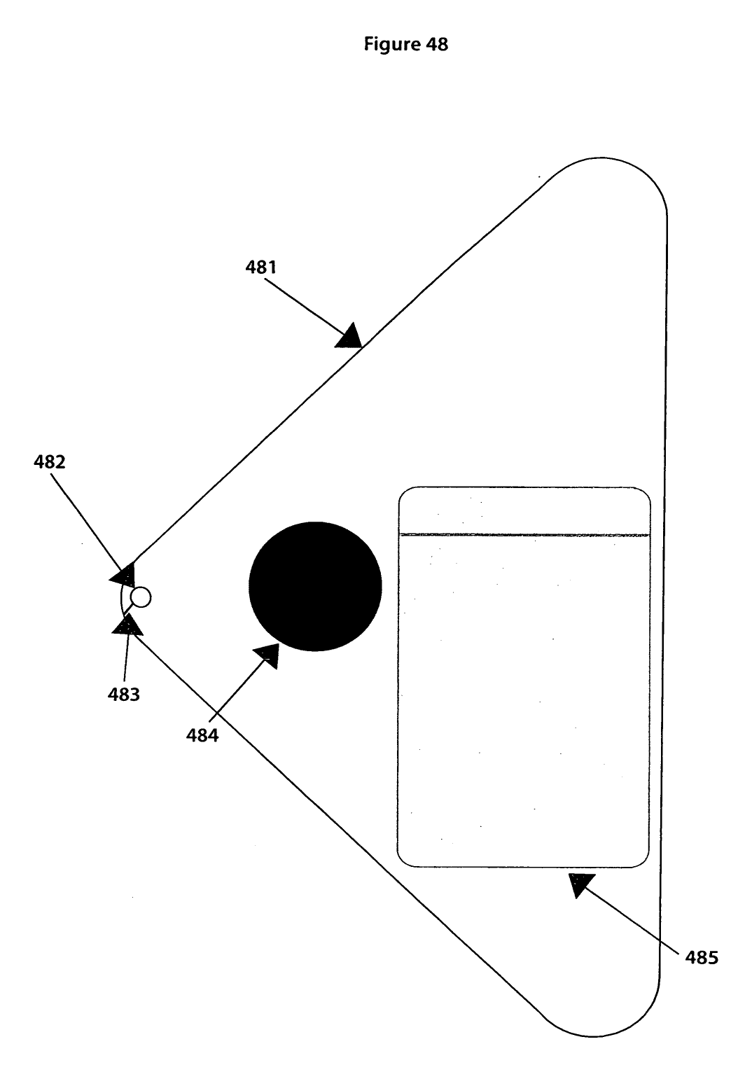

FIG. 49 is back view of a humming bird, Happy Hanukkah, and hope greeting cards with poly pocket, magnet, and aperture with easy release mechanism used for hanging purposes and/or attaching a decorative item like a tassel.

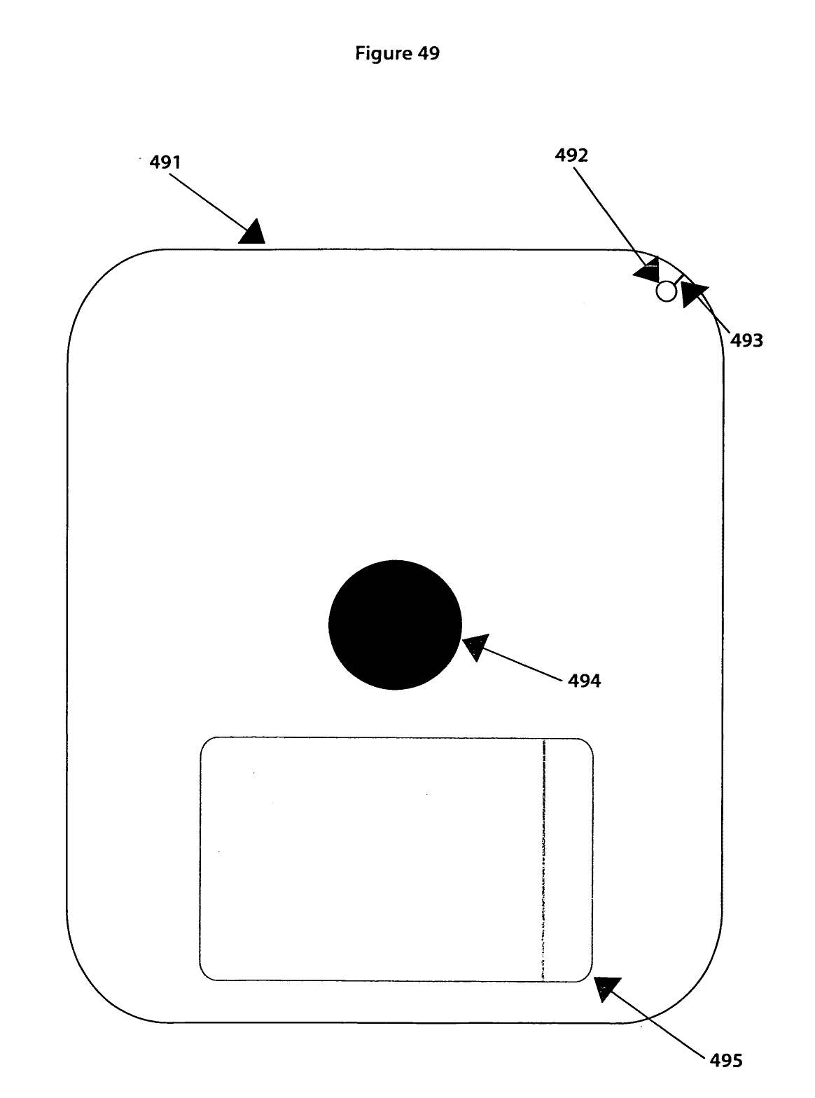

FIG. 50 is a back view of a Christmas tree greeting card with poly pocket, magnet, and aperture with easy release mechanism used for hanging purposes and/or attaching a decorative item like a tassel.

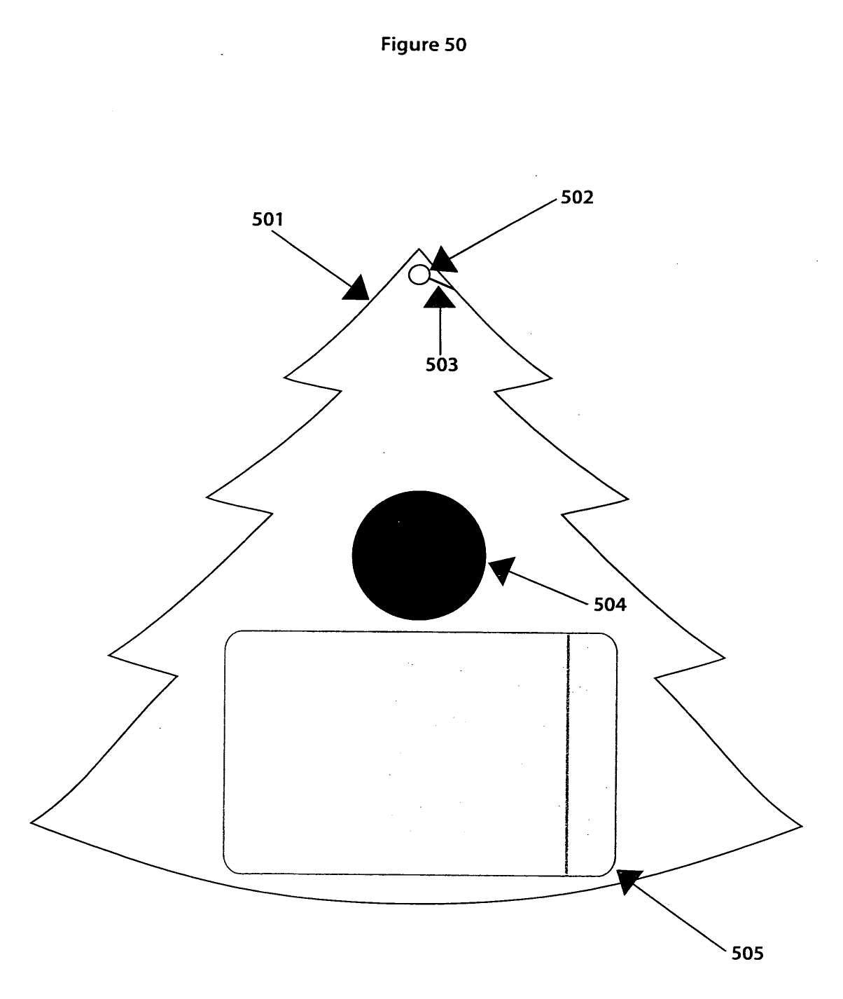

FIG. 51 is a back view of a Christmas snow-man greeting card with poly pocket, magnet, and aperture with easy release mechanism used for hanging purposes and/or attaching a decorative item like a tassel.

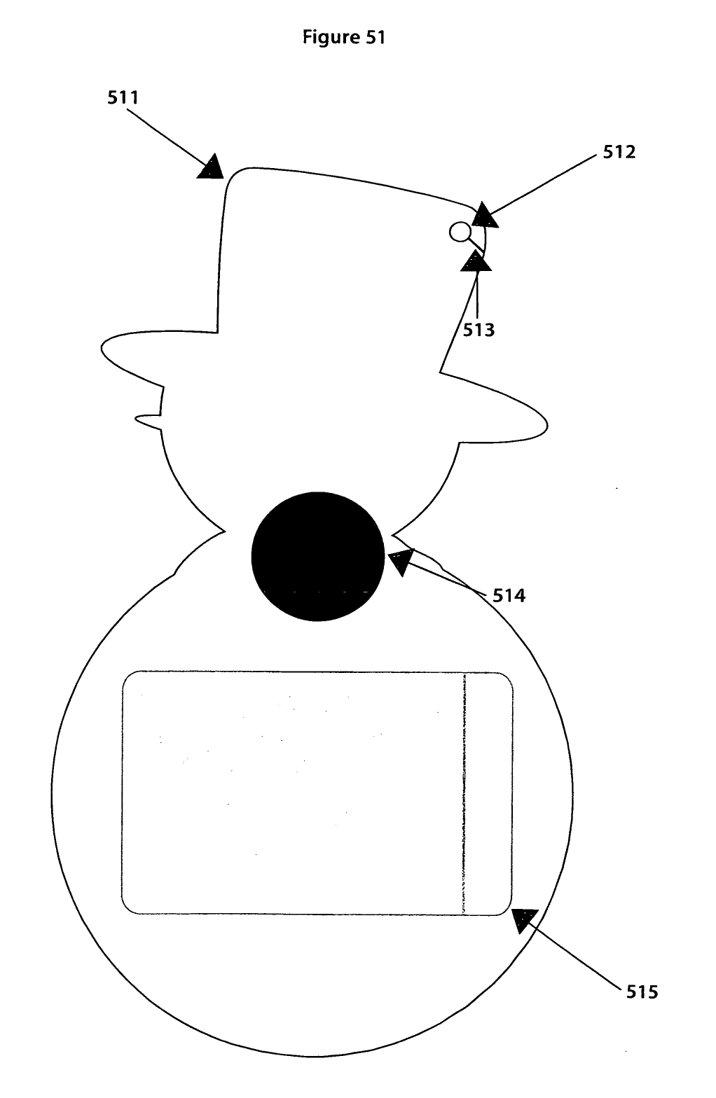

FIG. 52 is a back view of a Christmas coffee cup greeting card with poly pocket, magnet, and aperture with easy release mechanism used for hanging purposes and/or attaching a decorative item like a tassel.

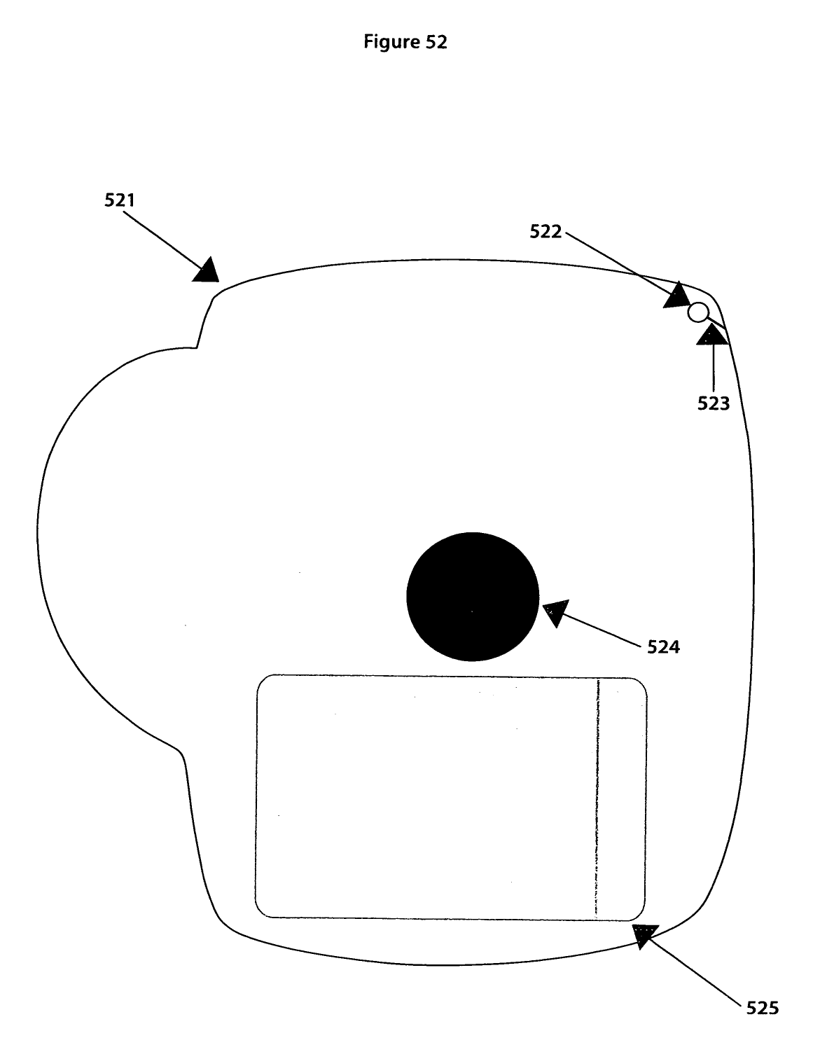

FIG. 53 is a back view of a Christmas church bell greeting card with poly pocket, magnet, and aperture with easy release mechanism used for hanging purposes and/or attaching a decorative item like a tassel.

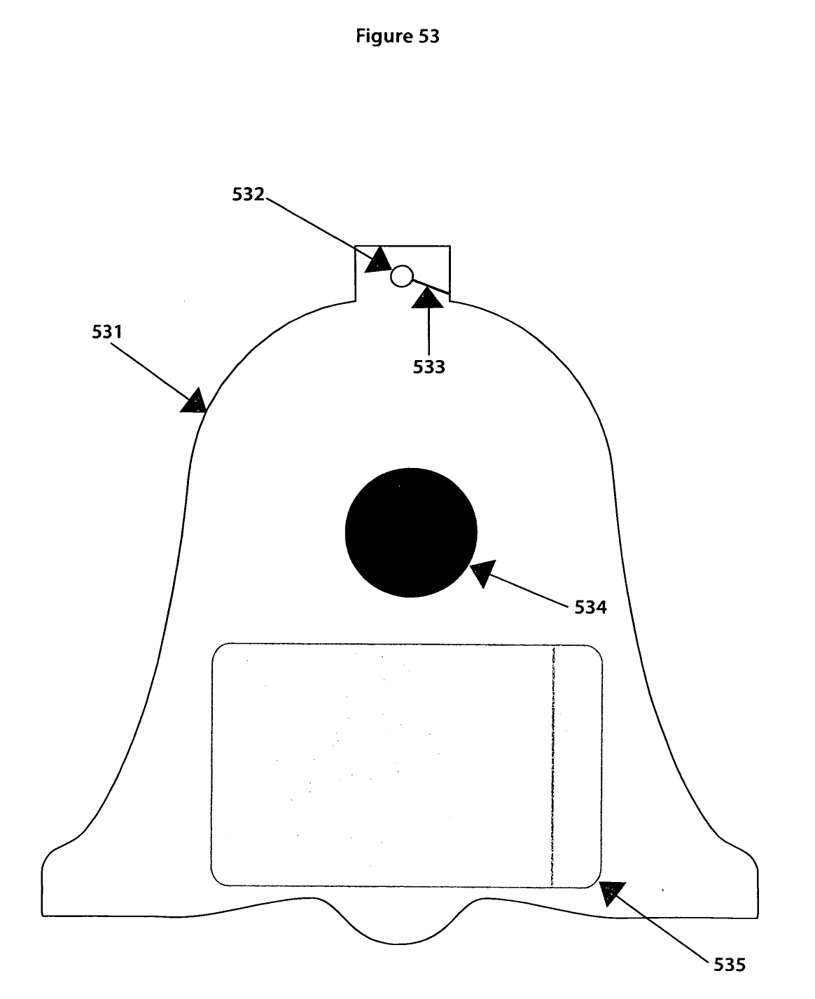

FIG. 54 is a with view of a Christmas angel greeting card with poly pocket, magnet, and aperture with easy release mechanism used for hanging purposes and/or attaching a decorative item like a tassel.

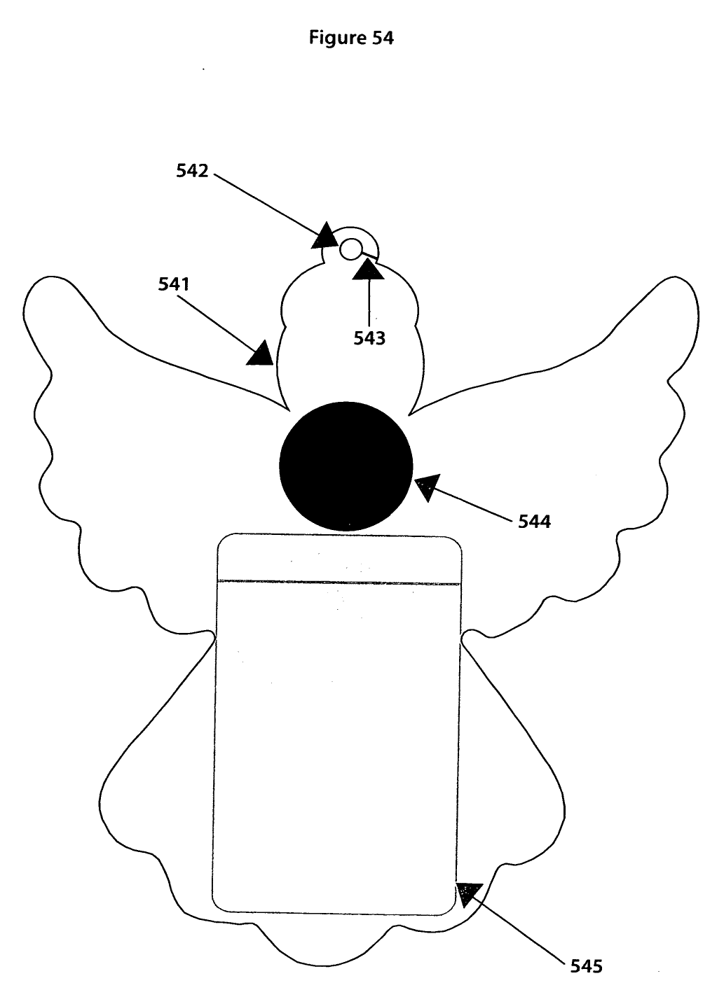

FIG. 55 is a back view of a Easter greeting card with poly pocket, magnet, and aperture with easy release mechanism used for hanging purposes and/or attaching a decorative item like a tassel.

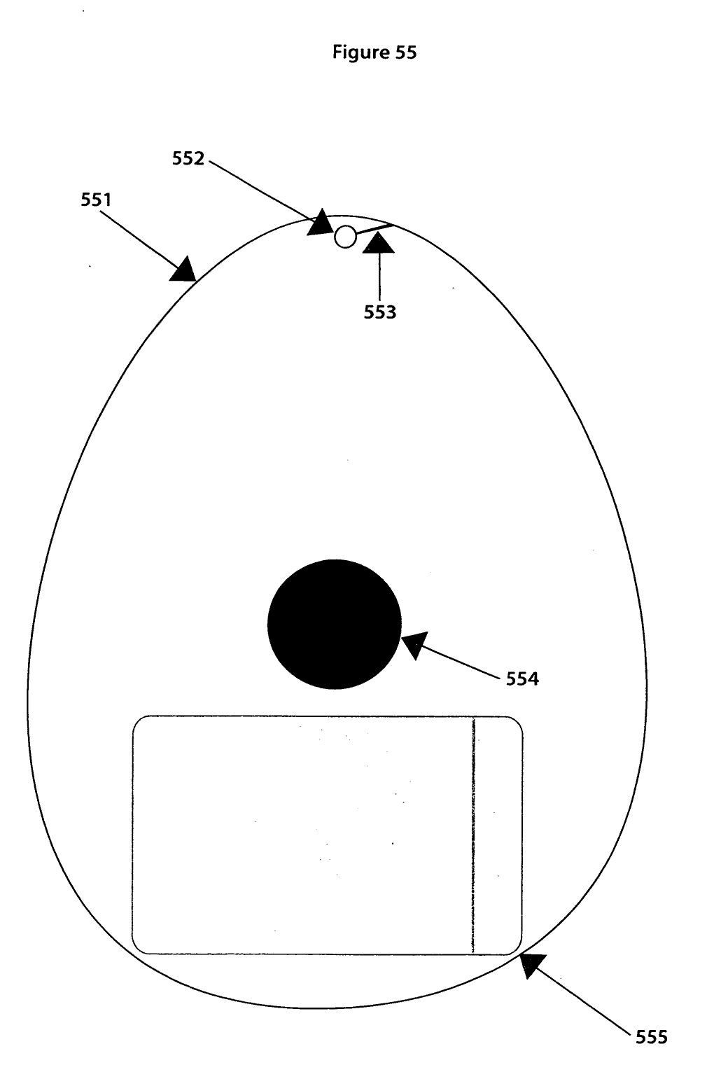

FIG. 56 is a back view of a leaf Christening blessing greeting card with poly pocket, magnet, and aperture with easy release mechanism used for hanging purposes and/or attaching a decorative item like a tassel.

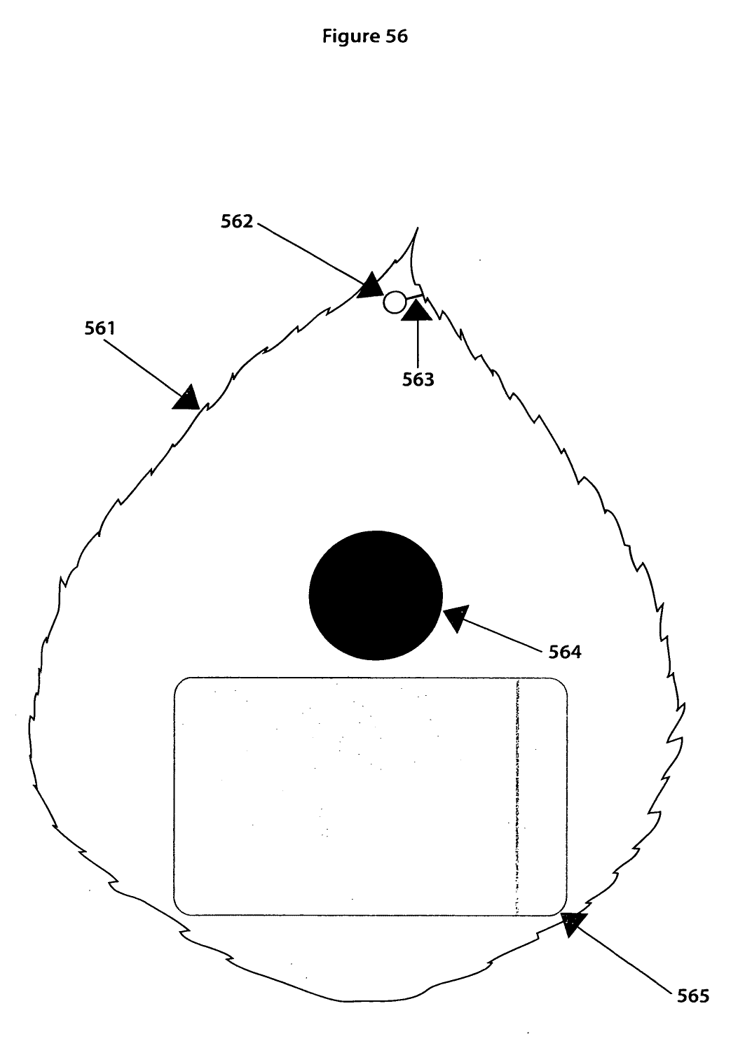

FIG. 57 is a back view of a “you can” encouragement greeting card with poly pocket, magnet, and aperture with easy release mechanism used for hanging purposes and/or attaching a decorative item like a tassel.

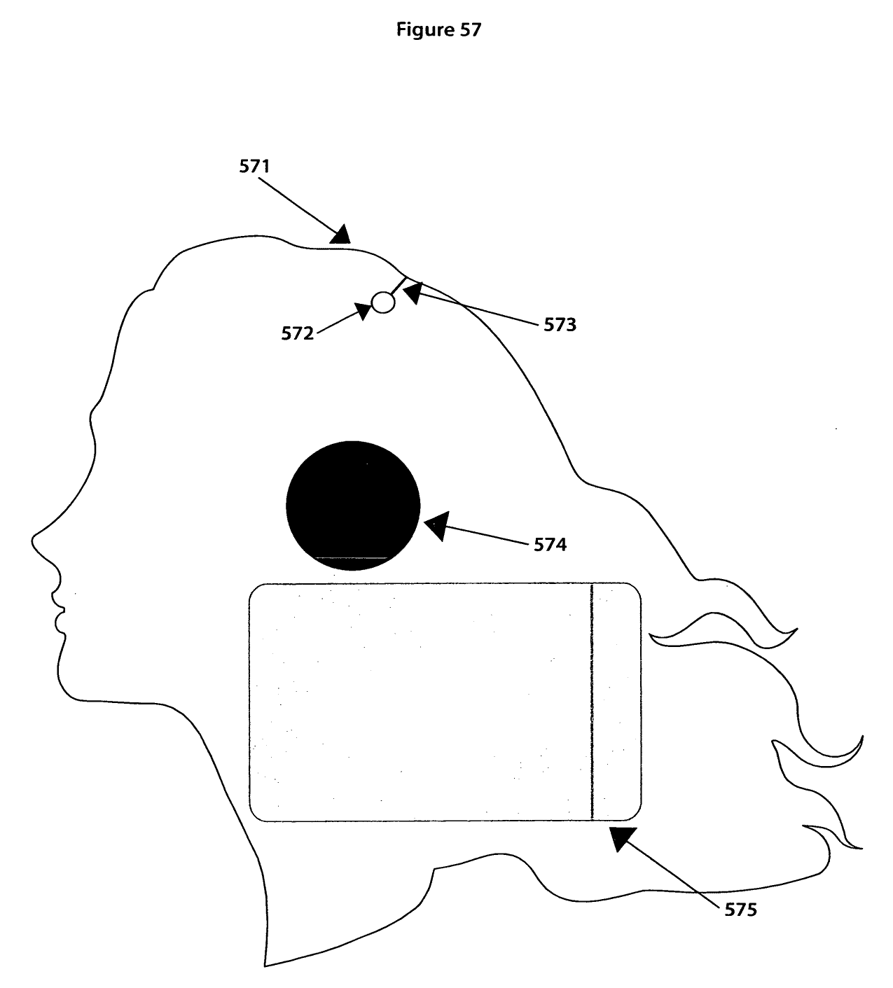

FIG. 58 is a back view of a mother's day giraffe greeting card with poly pocket, magnet, and aperture with easy release mechanism used for hanging purposes and/or attaching a decorative item like a tassel.

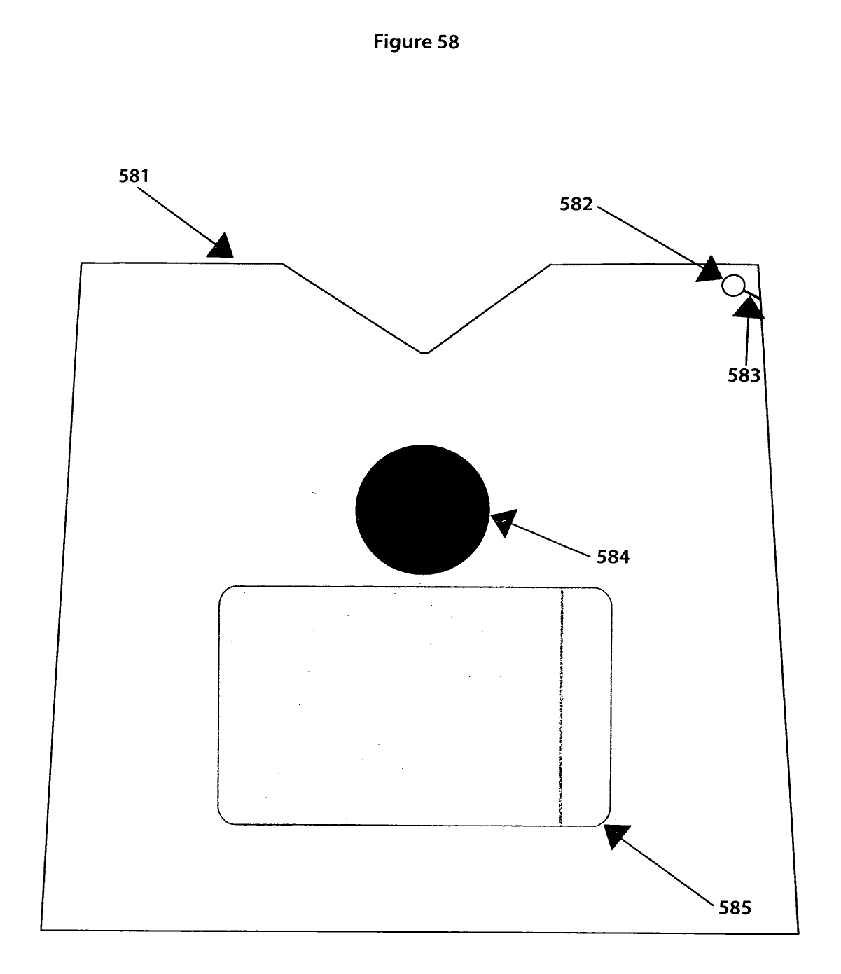

FIG. 59 is a back view of a doggy's new apartment greeting card with poly pocket, magnet, and aperture with easy release mechanism used for hanging purposes and/or attaching a decorative item like a tassel.

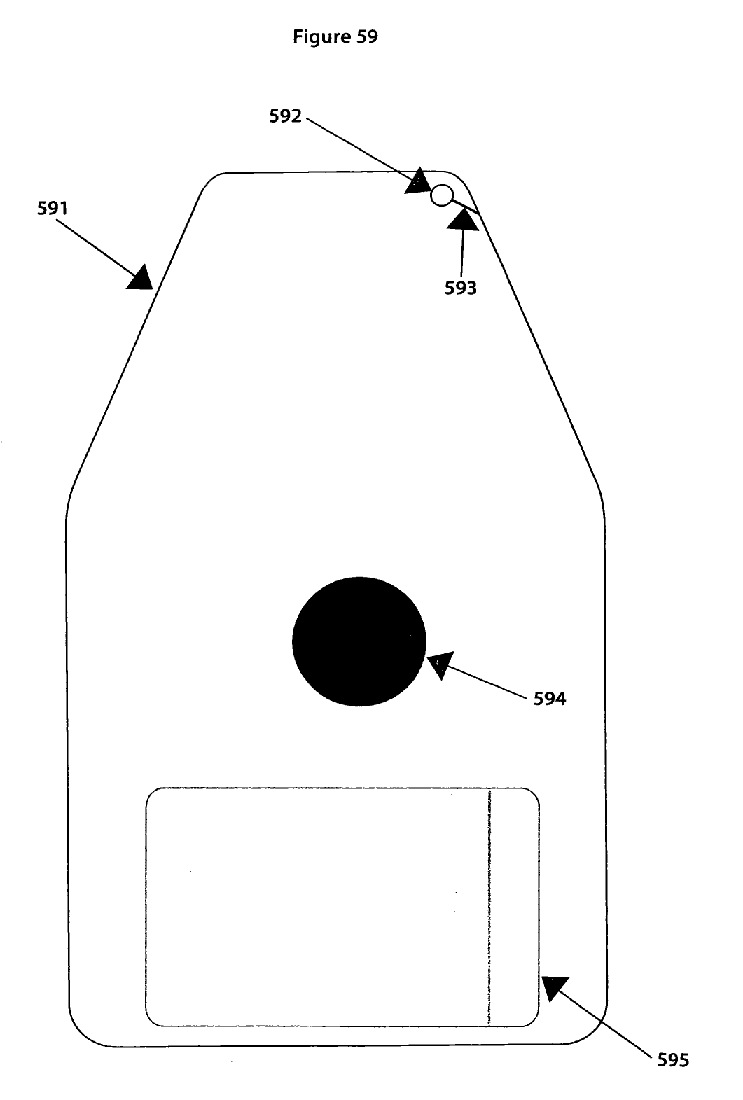

FIG. 60 is a back view of a Christmas stocking greeting card with poly pocket, magnet, and aperture with easy release mechanism used for hanging purposes and/or attaching a decorative item like a tassel.

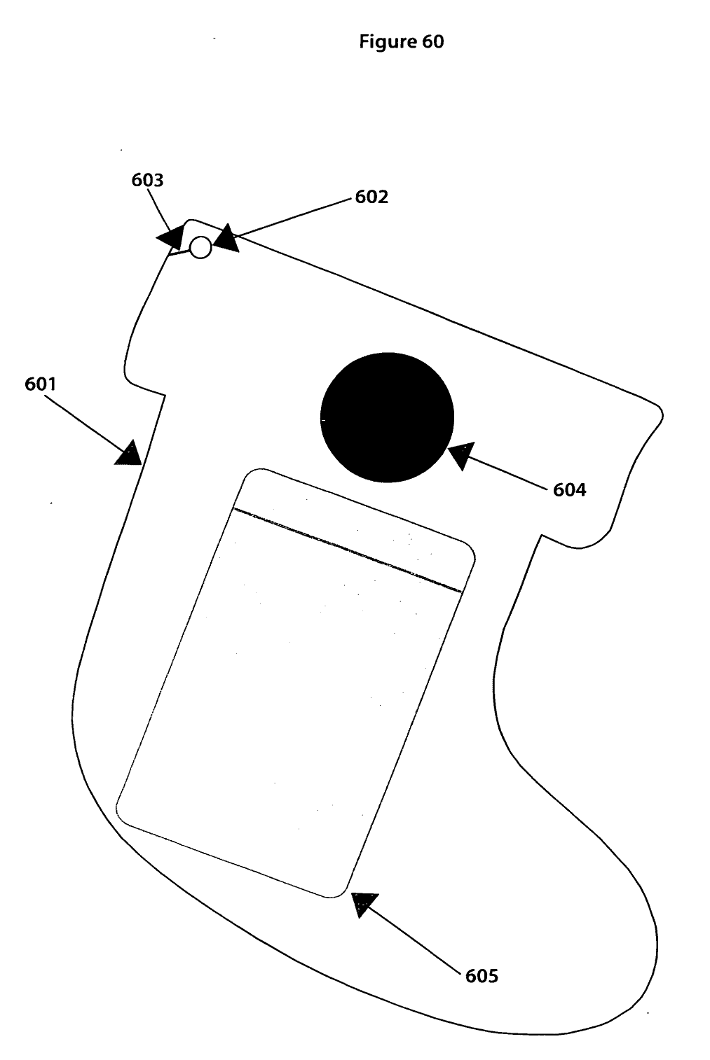

FIG. 61 is a back view of a bride and groom wedding celebration greeting card with poly pocket, magnet, and aperture with easy release mechanism used for hanging purposes and/or attaching a decorative item like a tassel.

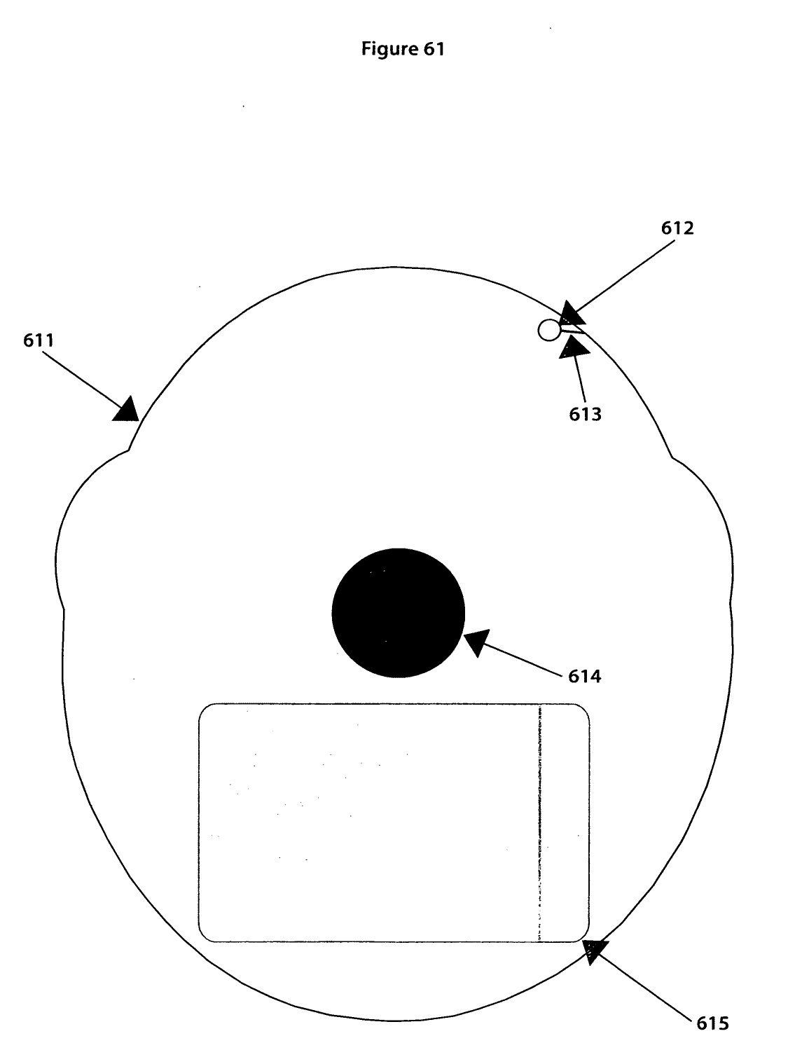

FIG. 62 is a back view of a basketball (T-Shirt) greeting card with poly pocket, magnet, and aperture with easy release mechanism used for hanging purposes and/or attaching a decorative item like a tassel.

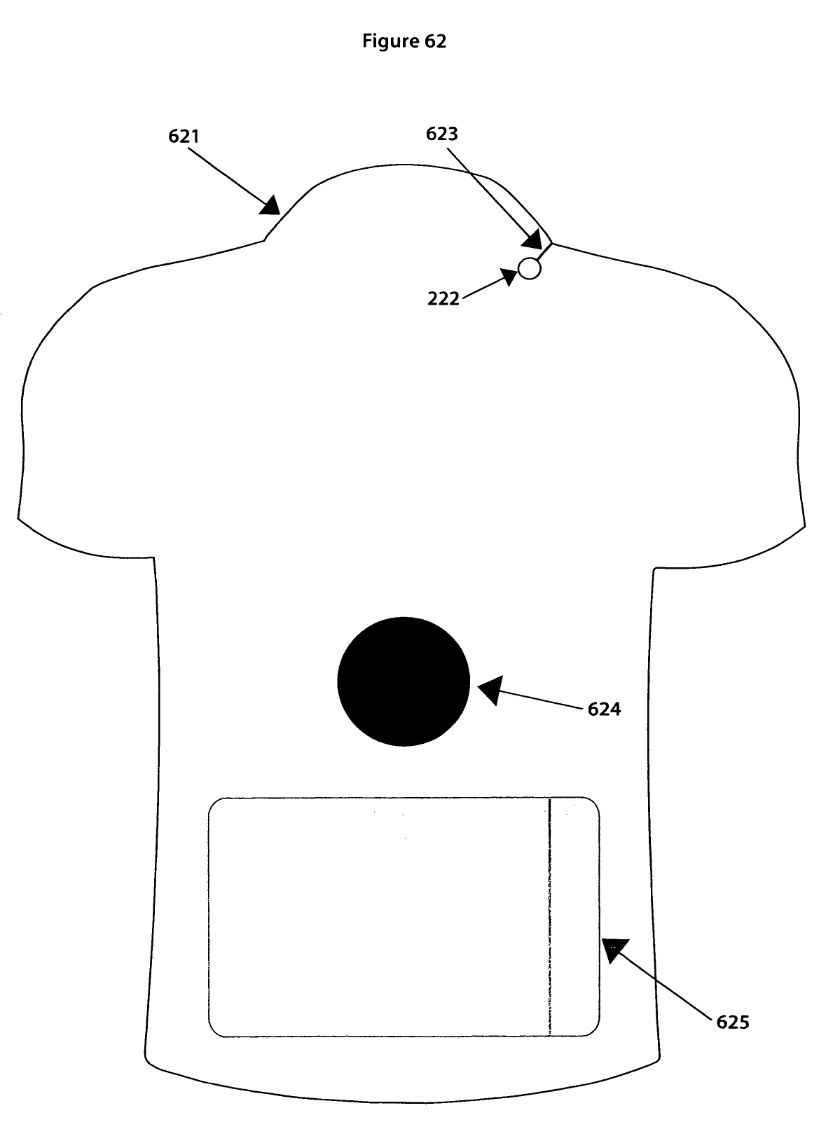

FIG. 63 is a back view of a birthday & Christmas invitation greeting cards with poly pocket, magnet, and aperture with easy release mechanism used for hanging purposes and/or attaching a decorative item like a tassel.

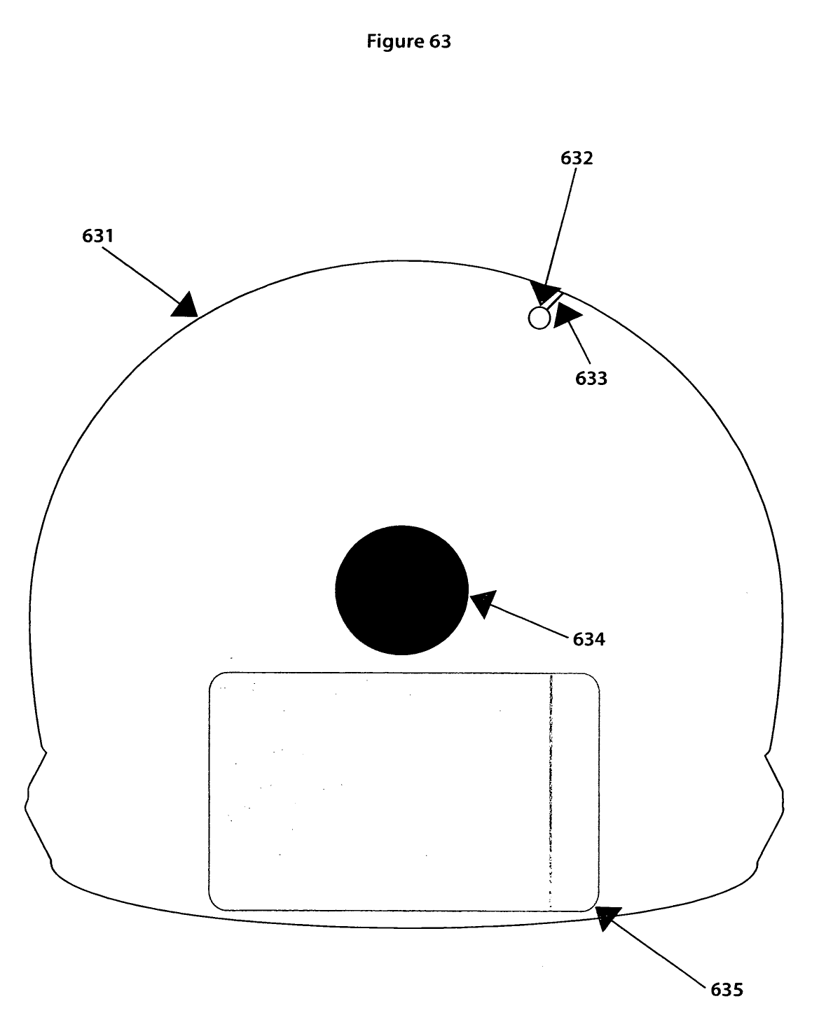

FIG. 64 is a back view of a hockey star greeting card with poly pocket, magnet, and aperture with easy release mechanism used for hanging purposes and/or attaching a decorative item like a tassel.

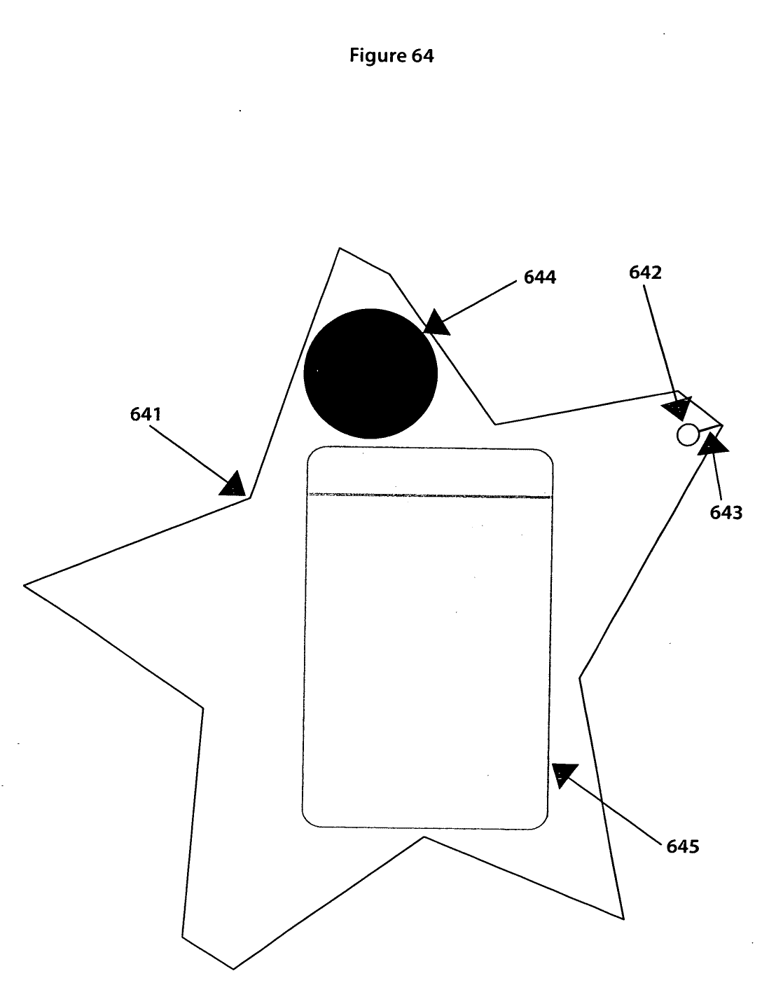

FIG. 65 is a back view of a stars-n-stripes Valentines greeting card with poly pocket, magnet, and aperture with easy release mechanism used for hanging purposes and/or attaching a decorative item like a tassel.

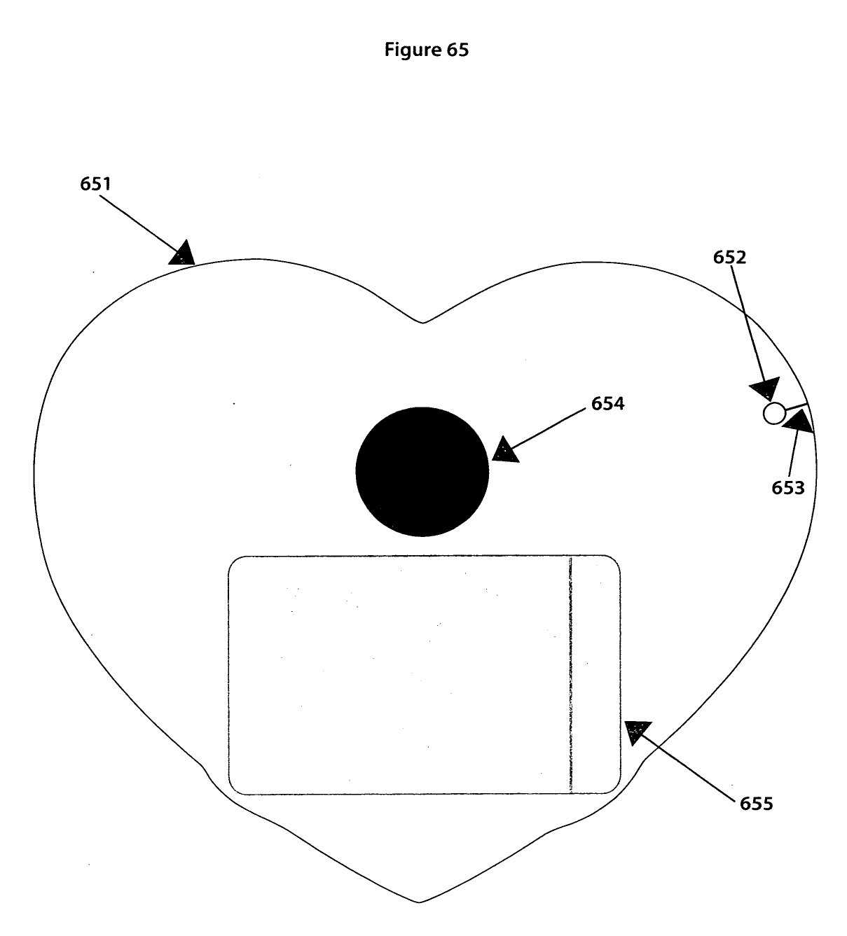

FIG. 66 is a back view of a lion(s) “I Love You” greeting card with poly pocket, magnet, and aperture with easy release mechanism used for hanging purposes and/or attaching a decorative item like a tassel.

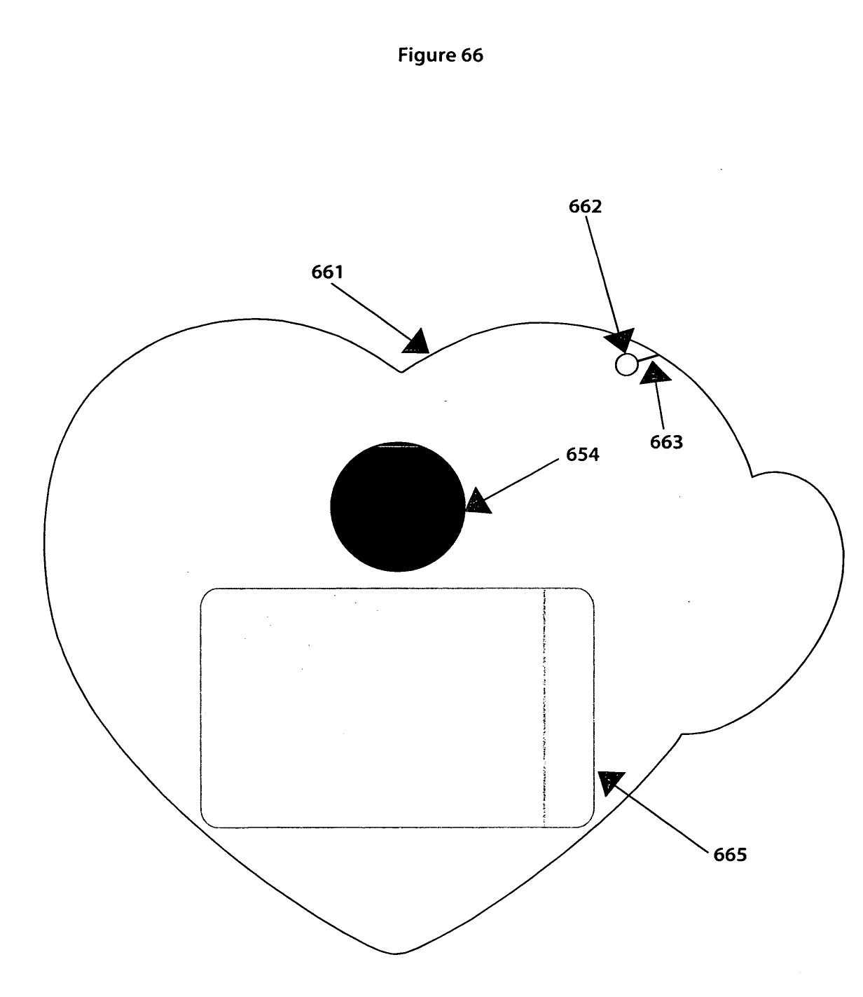

FIG. 67 is a back view of a pregnancy joy greeting card with poly pocket, magnet, and aperture with easy release mechanism used for hanging purposes and/or attaching a decorative item like a tassel.

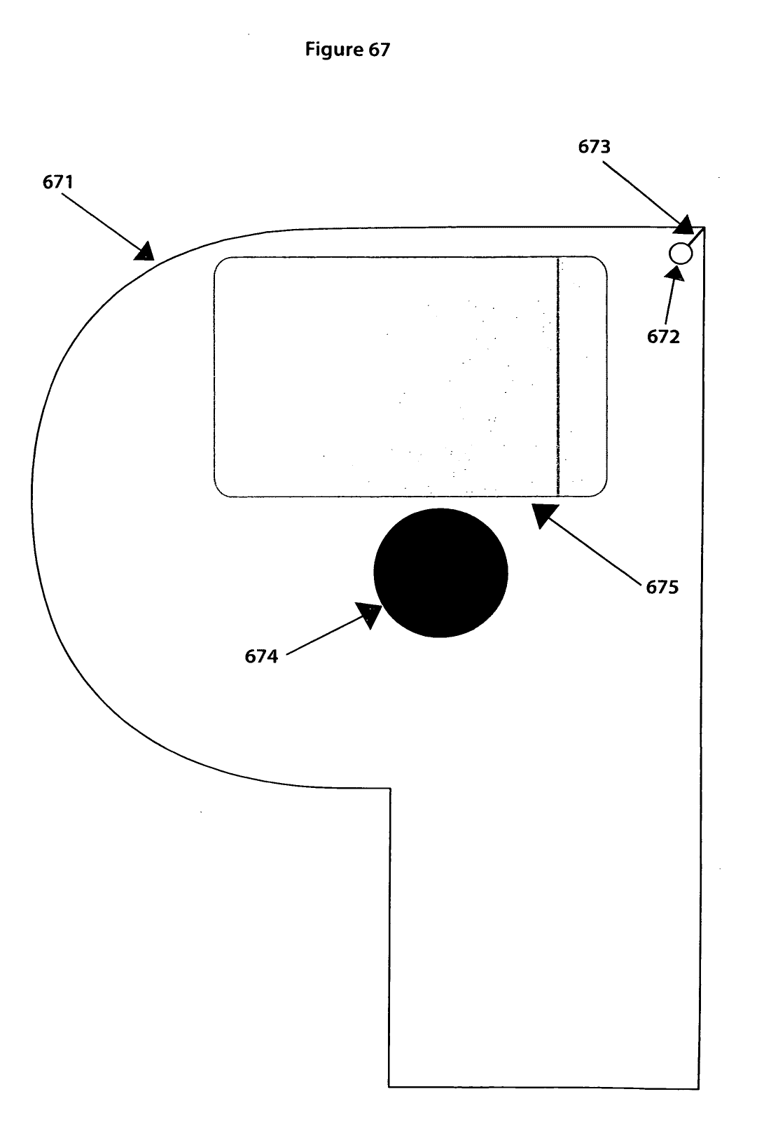

FIG. 68 is a back view of a her education greeting card with poly pocket, magnet, and aperture with easy release mechanism used for hanging purposes and/or attaching a decorative item like a tassel.

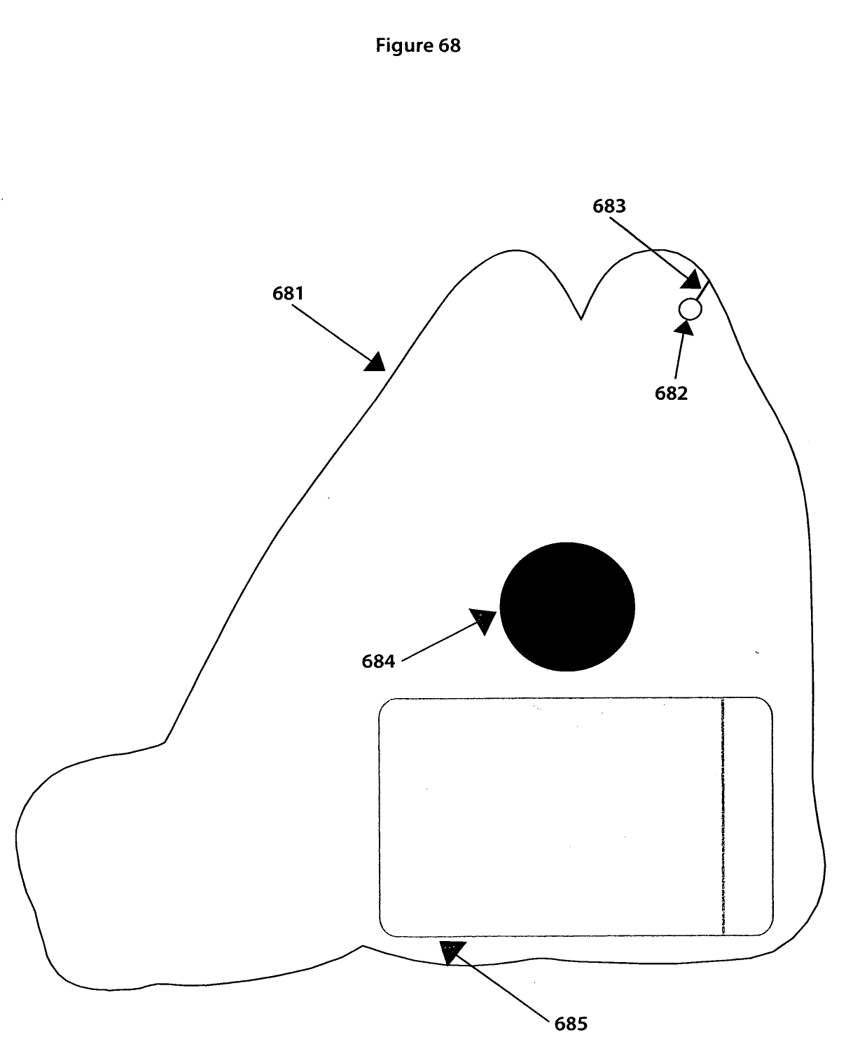

FIG. 69 is a back view of a puppy love-friendship greeting card with poly pocket, magnet, and aperture with easy release mechanism for hanging purposes and/or attaching a decorative item like a tassel.

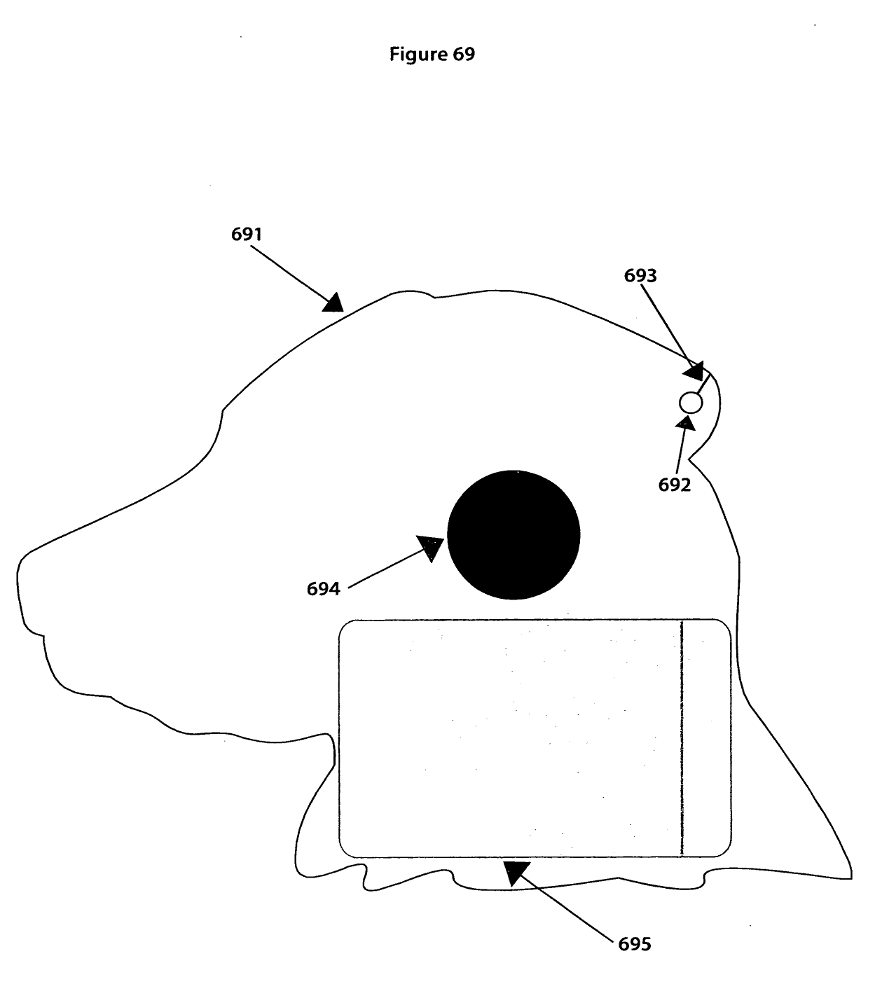

FIG. 70 is a back view of a thank you greeting card with poly pocket, magnet, and aperture with easy release mechanism used for hanging purposes and/or attaching a decorative item like a tassel.

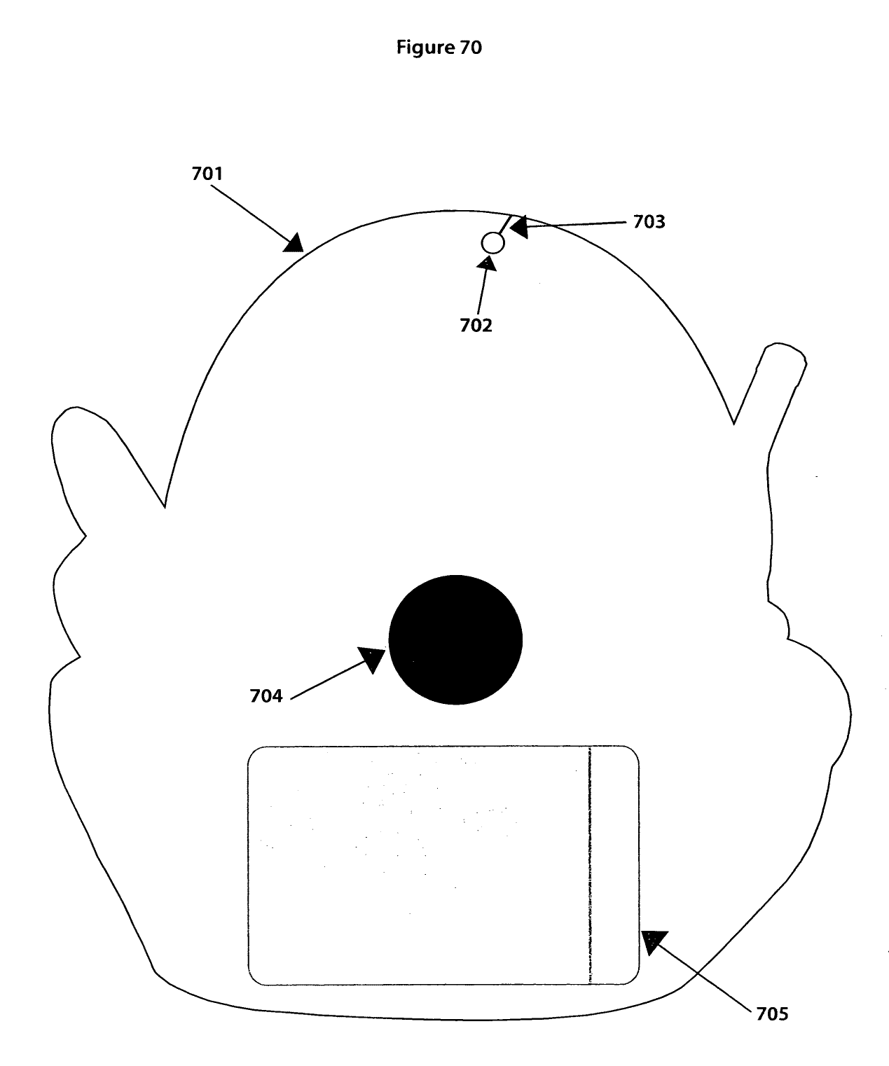

FIG. 71 is a back view of a sunflower greeting card with poly pocket, magnet, and aperture with easy release mechanism used for hanging purposes and/or attaching a decorative item like a tassel.

### DETAILED DESCRIPTION OF THE PREFERRED EMBODIMENTS

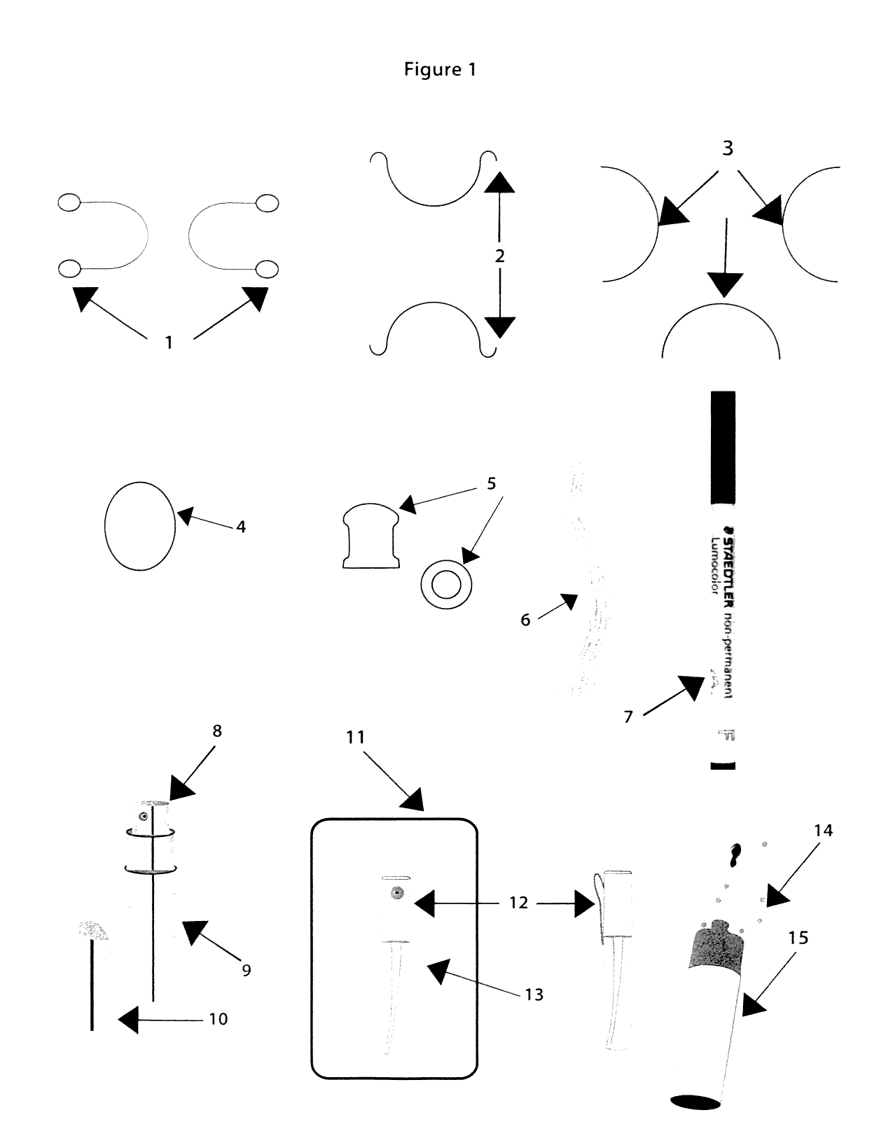

With reference now to the figures and in particular with reference to FIG. 1, a pictorial representation of several important components are shown. This illustrative embodiment first depicts a die-cut retention mechanism wherein two“U” shaped flaps face each other 1, and where each “U” gripping mechanism apertures as its' end points i.e. beginning and end of a line seen as a “U leg” and used for holding a pre-paid transaction card. Additionally shown in FIG. 1, is pair of die-cut retention mechanism 2 that have two half moon arc's (approximately 180 degrees) wherein the top of the arc of each acts as a retention mechanism and at its' base forms two smaller arcs shaped circles with arc openings inversely positioned to the greater opening positioned “center” and viewed as a half moom . . . Ultimately, 2 is an ideal die cut for providing an interference fit uniquely balancing the retention forces from the elongation of it's elastic body. A determination can be made as to positioning i.e. vertical, horizontal, or diagonal pending grain direction given a portrait or landscape artwork layout.

With regards to FIG. 1, item 3—depicts three arc mechanisms whereby a gift card is held in place in the same fashion as item 1 and 2. Item 4 depicts a decorative loop, book ring, or hook for attaching to a Christmas tree as an ornament with pre-paid gift card holder. Item 5 depicts a neodymium magnetic peg that is placed on the upper plane card surface directly above the magnet seen on the lower panel. This is especially beneficial and used to attach notes on said upper surface. Item 6 depicts a tassel which can be used as another attachment means to a Christmas tree and/or in an automobile. Item 7 shows a wet-erase or dry-erase “non-permanent” marker that can be utilized when an apparatus back surface is manufactured with a specific type of lamination (for example: Nylon or polypropelene, etc.) that is conducive to a non-permanent markert (example is wet or dry erase) which contains benefits of ecological re-use as in the case of a buiness application. For example, in hotels . . . writing down beverage and snack bar consumption might prove to be ideal. Item 8 depicts a reusable atomizer sprayer, item 9 shows a glue dot that can be utilized for holding a given atomizer on said apparatus, item 10 depicts a lid for the reusable sprayer. Item 11 is an external card, stock thickness grade that is used for advertising a specific fragrance brand or manufacturer.

Subsequent swap outs utilizing a “die-cut” retention mechanism makes for streamlined operations. Item 12 shows a non-reusable sprayer that is often used as“hand-out”sampler(s) in a department store. Item 13 shows a glue dot that is can be used for holding a sprayer onto a swap out card 11. Item 14 is a small “dog-tag” chain that can be used to attach a a canister that is more private for not openly disclosing a fragrance brand and is easily attached to the apparatus and/or tassel. And, item 15 is an external canister that can be utilized whereby a sprayer is placed inside, disclosed from an auto passenger(s) viewing.

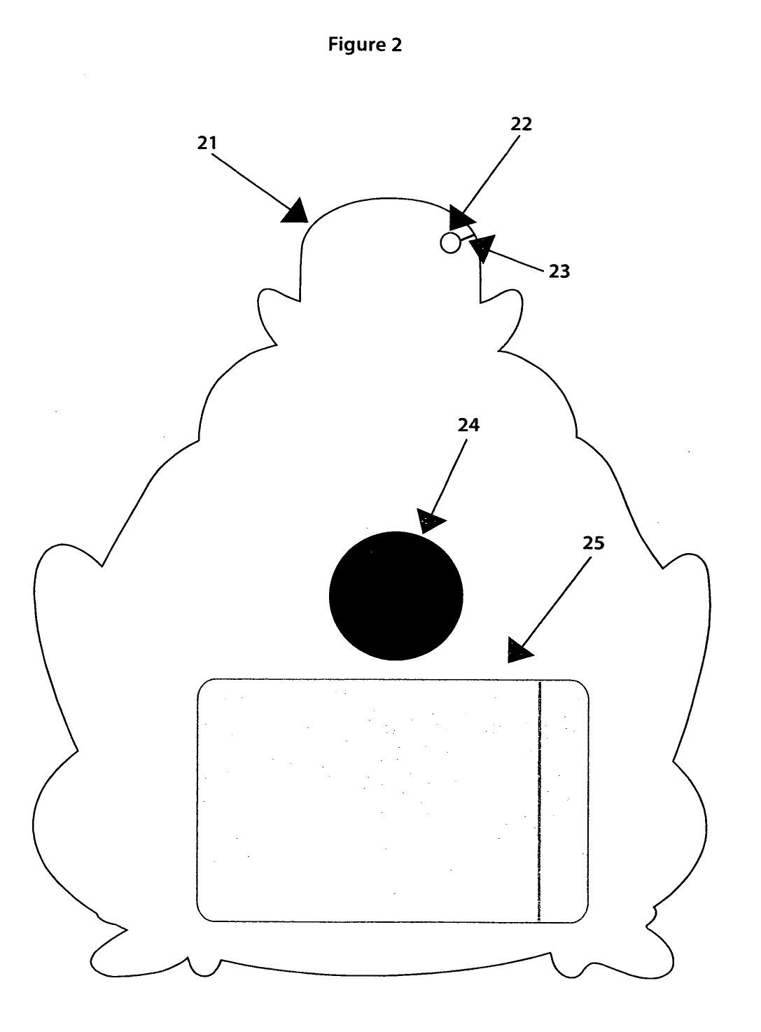

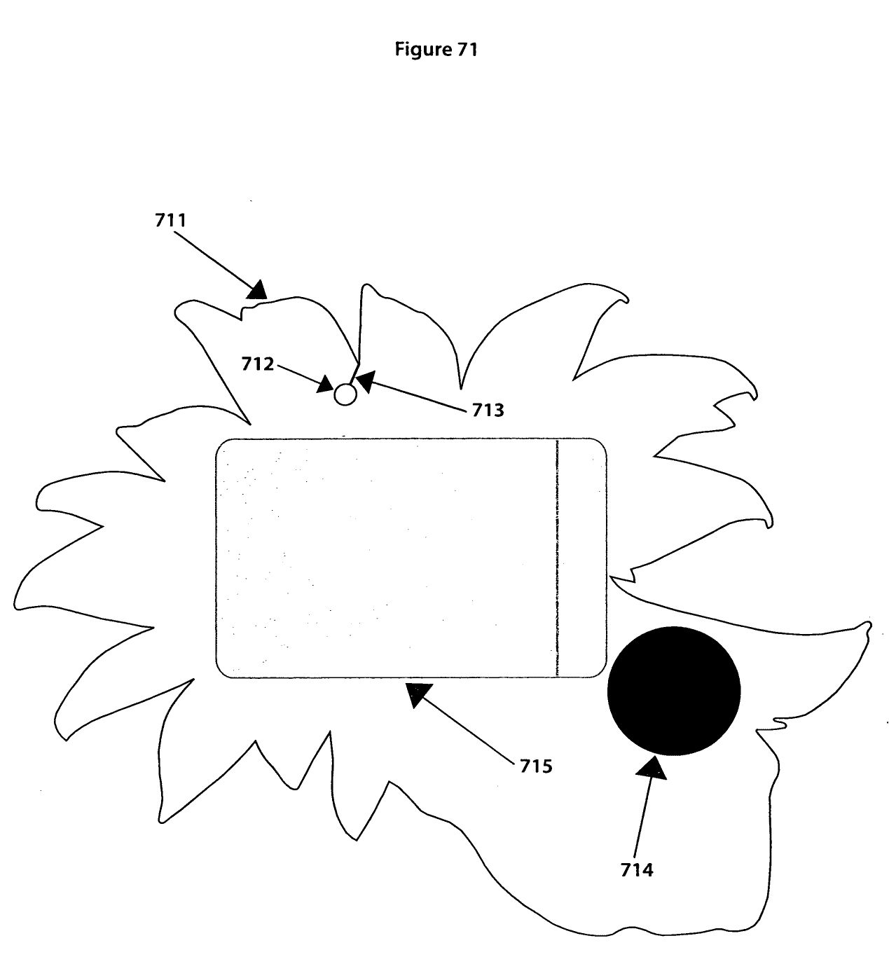

As shown in FIG. 2 through 71 . . . there are seventy different greeting card forms. This is seen by referencing: 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711.

As shown in FIG. 2 through 71 . . . an aperture is utilized (usually positioned somewhere near the top surface) for attaching a string, lanyard, or some other material thereto. This can be seen by turning to the aforementioned references: 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712.

As shown in FIG. 2 through 71 . . . is a die-cut slit typically positioned at approximately a 45 degree angle on left or right side of a given greeting card. The cut spans from the outer surface to aperture, thereby used for a easy attachement and/or quick release of string like component. This can be viewed by turning to the aforementioned figures and referencing the following: 13, 23, 33, 43, 53, 63, 73, 83, 93, 103, 113, 123, 133, 143, 153, 163, 173, 183, 193, 203, 213, 223, 243, 253, 263, 273, 283, 293, 303, 313, 323, 333, 343, 353, 363, 373, 383, 393, 403, 413, 423, 433, 443, 453, 463, 473, 483, 493, 503, 513, 523, 533, 543, 553, 563, 573, 583, 593, 603, 613, 623, 633, 643, 653, 663, 673, 683, 693, 703, and 713.

As shown in FIG. 2 through 71 . . . is a magnetic attachment placed on the backside of a greeting card and is utilized to facilitate attachment onto an appliance panel. This is seen by turning to the aforementioned figures and referencing: 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714.

As shown in FIG. 2 through 71 . . . is a poly pocked for inserting a pre-paid gift card. Typically positioned on the greeting cards back surface. This can be viewed by turning to the aforementioned figures and referencing the following: 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, and 715.

Thus, having set forth the preferred embodiments, the following is claimed:

1. A greeting card, comprising: 70 different greeting card shapes and sizes wherein having a front surface with a mascot motif theme and/or Christmas season design (with corresponding shapes). . . in addition to the integration of:
aperture—placed generally parallel to longitudinal upper side edges for attaching a string, tassel, hook; and
front surface base material that include a wide range of card stock and coatings, back surface material that enables writing on its surface which may include a synthetic or semi-synthetic lamination providing a smooth glass-like continuous area appearance which may be accomplished by utilizing polypropylene thermoplastic(s) or materials resembling a non-crystalline substance; a back surface conducive for writing a special occasion message; and
array of industry sizes (standard+custom) ranging between 3.25″-8.5″ in either length or width (landscape or portrait) with thicknesses ranging from approximately 1/64″ to 3/16″; A2-A7 as well as miscellaneous sizes that include: 4.25″×5.5″; 4.625″×6.26″, 5″×7; 6″×8; 5.5″×8.25, 5.25″×8.5″, 6″×9″; 7″×10; 7.75″×5.75 or numerous other combinations or permutations therein; and
translucent—poly plastic pocket attachment affixed (adhesive) to said apparatus front or back surface and capable of inserting a pre-paid gift card and/or a “collectible” sports card therein; and
various magnetic attachment pieces—shape(s) ranging from circles, squares, triangles . . . affixed to the apparatus back surface used to functionally adhere the apparatus to a kitchen appliance fixture (front panel).

2. The greeting card of claim 1, wherein a transaction card has a “retention mechanism” that is engineered from a die cut, incorporating either two or three half circular shapes . . . each half circular cut is adjoined with 2 smaller half circle cuts (approx. 3/16″wide) that begins at said “end-points” with openings inversely positioned to said larger half circular opening (approx. 1″); and may couple aperture(s) at the end of each die cut when using a “U” flap with end point—optimally configured for attaching a transaction gift card, flaps capable of bending 180 degrees upward or downward (predetermined bending length) in order to insert a gift card—said flaps acting as a gripping section—giving way to an interference fit so that when pressure is applied to the flap(s). . . the gift card is released from apparatus allowing pre-paid gift card to be removed.

3. The greeting card of claim 1, wherein a note can be inserted between the appliance panel and apparatus back panel magnet; or a neodymium magnet peg can be utilized for attaching important messages on the front surface when utilized in conjunction with exact location of the back surface magnetic attachment(s).

4. The greeting card of claim 1, wherein said base thickness corresponds with mounting multiple card stock thicknesses ranging from a minimum parent sheet thickness of 0.08-to a maximum of 0.24 card stock.

5. The greeting card of claim 4, wherein the front panel base thickness is approximately 0.24 point card stock thickness and is mounted to a back surface thickness of a similar thickness (preventing overall product material bend or warp) yielding 0.48 card thickness collectively and/or approximately 1/16″.

6. The greeting card of claim 1, wherein an envelope attachment is utilized as a means for disclosing said greeting card coupled with a pre-paid gift card.

7. The greeting card of claim 1 wherein, the card stock grain runs lengthwise (for most cards) to the long-side dimension (approximately 7.5-8″) pending poly pocket or die-cut spatial characterization . . . in order to optimize the bend of card as well as the die-cut flaps therein . . . streamlining insertion of pre-paid gift card.

8. The greeting card of claim 7, wherein a poly laminate can be combined with apparatus card stock thickness such that the fiber(s) renders additional rigidity facilitating a more sturdy writing platform.

9. The greeting card of claim 8, wherein a non-permanent marker may be utilized (wet or dry erase capability) providing an ecological re-usable hotel key card presenter and room/mini-bar consumption clipboard.

10. The greeting card of claim 1, wherein the front surface can utilize spot UV to accent and/or spotlight specific mascot motif features and/or UV flood coat to emphasize a collective design while providing water resistance.

11. The greeting card of claim 1, wherein indicia may be viewed on front surface indicating the occasion i.e. Happy Birthday, Thank You, Thinking of You.

12. The greeting card of claim 11, wherein the front or back surface may contain further enhancements that may include high impact decorative foiling, embossing, and/or de-bossing for echoing a particular sports brand or team logo.

13. A greeting card of claim 1, wherein small furniture (circular or square) pads (usually 3 or 4 utilizing felt material) are applied to the bottom of the invention thereby enabling the apparatus to be used as a beverage coaster.

14. A Christmas ornament with transaction pre-paid gift card holder comprising a main body consisting of:
card stock and/or polymeric material like Olefin, PVC, and Celetec high-temp plastic that may contain flametec fire resistant substrate; and
means for hanging to a Christmas tree utilizing an ornament loop or hook.

15. A Christmas ornament of claim 14, wherein a die cut line transverses between hook aperture and said longitudinal edges providing a quick release mechanism for moving ornament without removing tree loop, book ring and/or or hook from said holiday-display structure.

16. A Christmas ornament of claim 15, wherein indicia may be viewed on back surface indicating To:, From:, $ as well as a surface for writing a personal “good cheer” message.

17. An automobile, truck, boat etc. perfume-cologne atomizer card and dispersing method that consisting of:
various shapes and sizes capable of hanging/made of card stock or plastic; and
manufactured with multiple thicknesses; and
aperature coupled with die cut“quick removal” slit to outer edge as a means for hanging a piece of yarn or string to a vehicle interior panel knob (example: A/C or radio); and
die-cut/retention flap mechanism for inserting an external card; and
impregnated porous card stock used as a carrier adapted to discharge/dispense a substrate formulation compound composed of approximately 85%-90% fragrance oil coupled with 10%-15% hexylene glycol.

18. A car or truck perfume-cologne atomizer card of claim 17, wherein a refillable or non-refillable lightweight portable mist sprayer is attached to either the apparatus and/or a string, yarn, or tassel utilizing a “clip.”

19. A car or truck perfume-cologne atomizer of claim 17 wherein, a clear adhesive glue dot with high-tack permanent transparent adhesive (½″ diameter-⅛″ thick) is placed on the back surface of said apparatus and is used for attaching a non-refillable and/or refillable mist fragrance spray bottle thereon.

20. A car or truck perfume-cologne atomizer of claim 17 wherein, an external “blank business card” shape, made of card-stock(typically point 0.12-0.16 card stock and measuring approximately 2.15″×3.5″) provides an easy swap out of empty perfume and/or cologne container and can include promotional indicia from a specific fragrance manufacturer.
